SEQUENCE LISTING (131 sequences)

SEQ ID NO: 1 - protein, 10 aa, Artificial Sequence
  LCDR1
RASSGVNYMY

SEQ ID NO: 2 - protein, 7 aa, Artificial Sequence
  LCDR2
YTSILAP

SEQ ID NO: 3 - protein, 9 aa, Artificial Sequence
  LCDR3
QQFTSSPYT

SEQ ID NO: 4 - protein, 11 aa, Artificial Sequence
  LCDR1
RASQSIGTSIH

SEQ ID NO: 5 - protein, 7 aa, Artificial Sequence
  LCDR2
YGSESIS

SEQ ID NO: 6 - protein, 9 aa, Artificial Sequence
  LCDR3
QQSNTWPLT

SEQ ID NO: 7 - protein, 12 aa, Artificial Sequence
  LCDR1
SASSIIGSDFLH

SEQ ID NO: 8 - protein, 7 aa, Artificial Sequence
  LCDR2
RTSILAS

SEQ ID NO: 9 - protein, 9 aa, Artificial Sequence
  LCDR3
QQGSGLPLT

SEQ ID NO: 10 - protein, 11 aa, Artificial Sequence
  LCDR1
KASQDINSHLS

SEQ ID NO: 11 - protein, 7 aa, Artificial Sequence
  LCDR2
RANRLAD

SEQ ID NO: 12 - protein, 9 aa, Artificial Sequence
  LCDR3
LQYDEFPYT

SEQ ID NO: 13 - protein, 10 aa, Artificial Sequence
  LCDR1
SASSSVSYMY

SEQ ID NO: 14 - protein, 7 aa, Artificial Sequence
  LCDR2
LTSNLAS

SEQ ID NO: 15 - protein, 9 aa, Artificial Sequence
  LCDR3
QQWSGNPFT

SEQ ID NO: 16 - protein, 11 aa, Artificial Sequence
  LCDR1
RASENIYSYLT

SEQ ID NO: 17 - protein, 7 aa, Artificial Sequence
  LCDR2
NAKTLAE

SEQ ID NO: 18 - protein, 9 aa, Artificial Sequence
  LCDR3
QHHYGSPRT

SEQ ID NO: 19 - protein, 12 aa, Artificial Sequence
  LCDR1
SASSSISSNFLH

SEQ ID NO: 20 - protein, 7 aa, Artificial Sequence
  LCDR2
RTSILAS

SEQ ID NO: 21 - protein, 9 aa, Artificial Sequence
  LCDR3
QQGSGLPLT

SEQ ID NO: 22 - protein, 5 aa, Artificial Sequence
  LCDR1
SSVSY

SEQ ID NO: 23 - protein, 3 aa, Artificial Sequence
  LCDR2
DTS

SEQ ID NO: 24 - protein, 9 aa, Artificial Sequence
  LCDR3
QQWSSFPWT

SEQ ID NO: 25 - protein, 6 aa, Artificial Sequence
  LCDR1
EDIYDR

SEQ ID NO: 26 - protein, 3 aa, Artificial Sequence
  LCDR2
GTA

SEQ ID NO: 27 - protein, 9 aa, Artificial Sequence
  LCDR3
QQYWTTPWT

SEQ ID NO: 28 - protein, 5 aa, Artificial Sequence
  LCDR1
SSVNY

SEQ ID NO: 29 - protein, 3 aa, Artificial Sequence
  LCDR2
YTS

SEQ ID NO: 30 - protein, 9 aa, Artificial Sequence
  LCDR3
QQFTSSPFT

SEQ ID NO: 31 - protein, 7 aa, Artificial Sequence
  LCDR2
RANRLAT

SEQ ID NO: 32 - protein, 9 aa, Artificial Sequence
  LCDR3
QQYDEFPYT

SEQ ID NO: 33 - protein, 5 aa, Artificial Sequence
  HCDR1
KYWIE

SEQ ID NO: 34 - protein, 17 aa, Artificial Sequence
  HCDR2
EILPGSVITNYNEKFKG

SEQ ID NO: 35 - protein, 12 aa, Artificial Sequence
  HCDR3
WGLYDSDDGVDY

SEQ ID NO: 36 - protein, 5 aa, Artificial Sequence
  HCDR1
GCTMS

SEQ ID NO: 37 - protein, 17 aa, Artificial Sequence
  HCDR2
YISNGGDITYYPDTVKG

SEQ ID NO: 38 - protein, 10 aa, Artificial Sequence
  HCDR3
LDGYYYAMDF

SEQ ID NO: 39 - protein, 5 aa, Artificial Sequence
  HCDR1
SYVMH

SEQ ID NO: 40 - protein, 17 aa, Artificial Sequence
  HCDR2
YINPYNDGPKYNEKFKG

SEQ ID NO: 41 - protein, 12 aa, Artificial Sequence
  HCDR3
WDYFNSASGFAF

SEQ ID NO: 42 - protein, 5 aa, Artificial Sequence
  HCDR1
DYFLN

SEQ ID NO: 43 - protein, 17 aa, Artificial Sequence
  HCDR2
RINPYNGDSFINQNFRD

SEQ ID NO: 44 - protein, 13 aa, Artificial Sequence
  HCDR3
GGYDGYFIAYFDY

SEQ ID NO: 45 - protein, 5 aa, Artificial Sequence
  HCDR1
SYTMH

SEQ ID NO: 46 - protein, 17 aa, Artificial Sequence
  HCDR2
YINPTIGYTEYNQKFKD

SEQ ID NO: 47 - protein, 13 aa, Artificial Sequence
  HCDR3
LVITSVLGRAMDY

SEQ ID NO: 48 - protein, 5 aa, Artificial Sequence
  HCDR1
DYGVN

SEQ ID NO: 49 - protein, 17 aa, Artificial Sequence
  HCDR2
WVNTNTRESTYVEDFKG

SEQ ID NO: 50 - protein, 14 aa, Artificial Sequence
  HCDR3
GAYDAYYYYYGMDY

SEQ ID NO: 51 - protein, 5 aa, Artificial Sequence
  HCDR1
TYVMH

SEQ ID NO: 52 - protein, 17 aa, Artificial Sequence
  HCDR2
YINPNNDGPNYNEKFKG

SEQ ID NO: 53 - protein, 12 aa, Artificial Sequence
  HCDR3
WDSYNSAAGFAY

SEQ ID NO: 54 - protein, 8 aa, Artificial Sequence
  HCDR1
GFTLSTYT

SEQ ID NO: 55 - protein, 8 aa, Artificial Sequence
  HCDR2
ITSGDTYT

SEQ ID NO: 56 - protein, 8 aa, Artificial Sequence
  HCDR3
TRDRPLFH

SEQ ID NO: 57 - protein, 8 aa, Artificial Sequence
  HCDR1
GYTFTDYE

SEQ ID NO: 58 - protein, 8 aa, Artificial Sequence
  HCDR2
IHPGSGGT

SEQ ID NO: 59 - protein, 12 aa, Artificial Sequence
  HCDR3
TRAVSGYYAMDY

SEQ ID NO: 60 - protein, 8 aa, Artificial Sequence
  HCDR1
GYTFSNYL

SEQ ID NO: 61 - protein, 8 aa, Artificial Sequence
  HCDR2
IYPGDNNT

SEQ ID NO: 62 - protein, 11 aa, Artificial Sequence
  HCDR3
AGGTDYDGFAN

SEQ ID NO: 63 - protein, 12 aa, Artificial Sequence
  HCDR3
ARAVSGYYAMDY

SEQ ID NO: 64 - protein, 106 aa, Artificial Sequence
  Light Chain (VL) Variable Domain Sequence
ENVLTQSPAIMSASLGEKVTMSCRASSGVNYMYWYQQKSDASPKLLIYYTSILAPGVPARFSGSGSGNSYSLTISSMEGEDAATYYCQQFTSSPYTFGGGTKLEIK

SEQ ID NO: 65 - protein, 107 aa, Artificial Sequence
  Light Chain (VL) Variable Domain Sequence
DILLTQSPAILSVSPGERVSFSCRASQSIGTSIHWYQQRTNGSPRLLIKYGSESISGIPSRFSGSGSGTDFTLSINSVESEDIADYYCQQSNTWPLTFGDGTKLELK

SEQ ID NO: 66 - protein, 108 aa, Artificial Sequence
  Light Chain (VL) Variable Domain Sequence
EIVLTQSPTTMAASPGEKITIICSASSIIGSDFLHWYQQRPGFSPKFLIYRTSILASGVPTRFTGSGSGTSYSLTIGTMEAEDVATYYCQQGSGLPLTFGSGTKLEMK

SEQ ID NO: 67 - protein, 107 aa, Artificial Sequence
  Light Chain (VL) Variable Domain Sequence
DIKLTQSQSSMYSSLGQRVTITCKASQDINSHLSWFQEKPGKSPKTLIYRANRLADGVPSRFSGSGSGQDYFLTISSLEYEDVGIYYCLQYDEFPYTFGGGTKLEIK

SEQ ID NO: 68 - protein, 106 aa, Artificial Sequence
  Light Chain (VL) Variable Domain Sequence
QIVLTQSPALMSASPGEKVTMTCSASSSVSYMYWFQQKPRSSPKPWIYLTSNLASGVPARFSGSGSGTSYSLTISSMEAEDAATYYCQQWSGNPFTFGSGTKLEIK

SEQ ID NO: 69 - protein, 107 aa, Artificial Sequence
  Light Chain (VL) Variable Domain Sequence
DIQMTQSPASLSASVGETVTITCRASENIYSYLTWYKQKQGKSPQLLVYNAKTLAEGVPSRFSGSGSGTQFSLKINSLQPEDFGSYYCQHHYGSPRTFGGGTKLEIK

SEQ ID NO: 70 - protein, 108 aa, Artificial Sequence
  Light Chain (VL) Variable Domain Sequence
EIVLTQSPTTMAASPGEKITIICSASSSISSNFLHWYQQKPGFSPRFLIYRTSILASGVPTRFSGSGSGTSYSLTIDTMEAEDVATYYCQQGSGLPLTFGSGTKLEIK

SEQ ID NO: 71 - protein, 106 aa, Artificial Sequence
  Light Chain (VL) Variable Domain Sequence
QIVLTQSPAIMSASPGEKVTMTCSASSSVSYMYWYQQKPGSSPRLLIYDTSNLASGVPVRFSGSGSGTSYSLTISRMEAEDAATYYCQQWSSFPWTFGGGTKLEIK

SEQ ID NO: 72 - protein, 107 aa, Artificial Sequence
  Light Chain (VL) Variable Domain Sequence
DIQMTQSSSSFSGSLGDRLTINCKASEDIYDRVAWYQQKPGNAPRLLISGTASLETGVLSRFSGSGSGKDYTLSINGLQAEDVATYYCQQYWTTPWTFGGGTKLEIK

SEQ ID NO: 73 - protein, 106 aa, Artificial Sequence
  Light Chain (VL) Variable Domain Sequence
ENVLTQSPAIMSASLGEKVTMSCRASSSVNYMYWYQQKSDASPKLWIYYTSKLAPGVPARFSGSGSGNSYSLTISSMEGEDAATYYCQQFTSSPFTFGSGTKLEIK

SEQ ID NO: 74 - protein, 107 aa, Artificial Sequence
  Light Chain (VL) Variable Domain Sequences
DIQMTQSPSSLSASVGDRVTITCKASQDINSHLSWYQQKPGKAPKLLIYRANRLATGVPSRFSGSGSGTDFTFTISSLQPEDIATYYCLQYDEFPYTFGGGTKLEIK

SEQ ID NO: 75 - protein, 107 aa, Artificial Sequence
  Light Chain (VL) Variable Domain Sequence
DIQMTQSPSSLSASVGDRVTITCKASQDINSHLSWYQQKPGKAPKLLIYRANRLATGVPSRFSGSGSGTDFTFTISSLEYEDIATYYCLQYDEFPYTFGGGTKLEIK

SEQ ID NO: 76 - protein, 107 aa, Artificial Sequence
  Light Chain (VL) Variable Domain Sequence
DIQMTQSPSSLSASVGDRVTITCKASQDINSHLSWYQQKPGKAPKLLIYRANRLATGVPSRFSGSGSGTDFTFTISSLQPEDIATYYCQQYDEFPYTFGGGTKLEIK

SEQ ID NO: 77 - protein, 107 aa, Artificial Sequence
  Light Chain (VL) Variable Domain Sequence
DIKMTQSPSSMYASLGQRVTITCKASQDINSHLSWFQEKPGKSPKTLIYRANRLADGVPSRFSGSGSGQDYFLTISSLEYEDVGIYYCLQYDEFPYTFGGGTKLEIK

SEQ ID NO: 78 - protein, 107 aa, Artificial Sequence
  Light Chain (VL) Variable Domain Sequence
DIQMTQSPSSLSASVGDRVTITCKASEDIYDRVAWYQQKPGKAPKLLIYGTASLETGVPSRFSGSGSGTDFTFTISSLQPEDIATYYCQQYWTTPWTFGGGTKVEIK

SEQ ID NO: 79 - protein, 107 aa, Artificial Sequence
  Light Chain (VL) Variable Domain Sequence
DIQMTQSPSSLSASVGDRVTITCKASEDIYDRVAWYQQKPGKAPKLLIYGTASLETGVPSRFSGSGSGTDFTLTISSLQPEDFATYYCQQYWTTPWTFGGGTKVEIK

SEQ ID NO: 80 - protein, 107 aa, Artificial Sequence
  Light Chain (VL) Variable Domain Sequence
DIQMTQSPSSLSASVGDRVTITCKASEDIYDRVAWYQQKPGKAPKLLIYGTASLETGVLSRFSGSGSGTDFTLTISSLQAEDFATYYCQQYWTTPWTFGGGTKVEIK

SEQ ID NO: 81 - protein, 121 aa, Artificial Sequence
  Heavy Chain (VH) Variable Domain Sequence
QVQLQQSGAELMKPGASVKISCKATGYSFTKYWIEWVKQRPGHGLEWIGEILPGSVITNYNEKFKGKATFTADTSSNTVYMQLSSLTSEDSAVYYCTKWGLYDSDDGVDYWGQGTTLTVSS

SEQ ID NO: 82 - protein, 119 aa, Artificial Sequence
  Heavy Chain (VH) Variable Domain Sequence
EVKLVESGGGLVQPGGSLKLSCAASGFSFSGCTMSWIRQTPERRLEWVAYISNGGDITYYPDTVKGRFTISRDNAKNSLYLQMSSLKSEDTAMYYCARLDGYYYAMDFWGQGTSVTVSS

SEQ ID NO: 83 - protein, 121 aa, Artificial Sequence
  Heavy Chain (VH) Variable Domain Sequence
EVQLQQSGPEVVKPGASVKMSCKASGYTFTSYVMHWVKQKPGQGLEWIGYINPYNDGPKYNEKFKGKATLTSDKSSSTAYMELSSLTSEDSAVYFCARWDYFNSASGFAFWGQGTLVTVSA

SEQ ID NO: 84 - protein, 122 aa, Artificial Sequence
  Heavy Chain (VH) Variable Domain Sequence
EVQLQQSGPDLVKPGASVKISCKASGYSFTDYFLNWVKQSHGKSLEWIGRINPYNGDSFINQNFRDKATLTVDKSSTTAHMDLLSLTSEDSAIYYCGRGGYDGYFIAYFDYWGQGSLVTVSA

SEQ ID NO: 85 - protein, 122 aa, Artificial Sequence
  Heavy Chain (VH) Variable Domain Sequence
QVQLQQSAAELARPGASVKMSCKASGYTFTSYTMHWVKQRPGQGLEWIGYINPTIGYTEYNQKFKDKTTLTADKSSSTAYMQLSSLTSEDSAVYYCVRLVITSVLGRAMDYWGQGTSVTVSS

SEQ ID NO: 86 - protein, 123 aa, Artificial Sequence
  Heavy Chain (VH) Variable Domain Sequence
QIQLVQSGPELKKPGETVKISCKASGYTFTDYGVNWVKQGPGKDLQWMGWVNTNTRESTYVEDFKGRFAFSLETSASTAYLQINNLKNEDSSTYFCARGAYDAYYYYYGMDYWGQGTSVTVSS

SEQ ID NO: 87 - protein, 121 aa, Artificial Sequence
  Heavy Chain (VH) Variable Domain Sequence
EVQLQQSGPELVKPGASVKMSCRASGYTFSTYVMHWIKHRPGQGLEWIGYINPNNDGPNYNEKFKGKATLTSDISSSTAYMELSSLTSEDSAVYFCSRWDSYNSAAGFAYWGHGTLVTVSA

SEQ ID NO: 88 - protein, 115 aa, Artificial Sequence
  Heavy Chain (VH) Variable Domain Sequence
EVQLQESGGGLVKPGGSLKLSCAASGFTLSTYTMSWVRQTPEKRLEWVAIITSGDTYTYYPDSVKGRFTISRDNAKNTLYLQMSSLKSEDTGMYYCTRDRPLFHWGQGTTLTVST

SEQ ID NO: 89 - protein, 119 aa, Artificial Sequence
  Heavy Chain (VH) Variable Domain Sequence
EVQLQESGAELVRPGASVKLSCKALGYTFTDYEIHWVKETPVYGLEWIGDIHPGSGGTANNQKFKGKATLTADKSSNTAYMELSSLTSEDSAVYYCTRAVSGYYAMDYWGQGTSVTVSS

SEQ ID NO: 90 - protein, 118 aa, Artificial Sequence
  Heavy Chain (VH) Variable Domain Sequence
EVQLQESGAELVRPGTSVKMSCKAAGYTFSNYLIGWIKQRPGHGLEWIGDIYPGDNNTNYNEKFRVKATLTADTSSNTAYMHLTSLTSEDSAIYYCAGGTDYDGFANWGQGTLVTVSA

SEQ ID NO: 91 - protein, 122 aa, Artificial Sequence
  Heavy Chain (VH) Variable Domain Sequence
QVQLVQSGAEVKKPGASVKVSCKASGYSFTDYFLNWVRQAPGQGLEWMGRINPYNGDSFINQNFRDRVTMTRDTSTSTVYMELSSLRSEDTAVYYCARGGYDGYFIAYFDYWGAGTTVTVSS

SEQ ID NO: 92 - protein, 122 aa, Artificial Sequence
  Heavy Chain (VH) Variable Domain Sequence
QVQLVQSGAEVKKPGSSVKVSCKASGYSFTDYFLNWVRQAPGQGLEWMGRINPYNGDSFINQNFRDRVTITADKSTSTAYMELSSLRSEDTAVYYCARGGYDGYFIAYFDYWGAGTTVTVSS

SEQ ID NO: 93 - protein, 122 aa, Artificial Sequence
  Heavy Chain (VH) Variable Domain Sequence
EVQLVQSGAEVKKPGESLKISCKGSGYSFTDYFLNWVRQMPGKGLEWMGRINPYNGDSFINQNFRDQVTISADKSISTAYLQWSSLKASDTAMYYCARGGYDGYFIAYFDYWGAGTTVTVSS

SEQ ID NO: 94 - protein, 122 aa, Artificial Sequence
  Heavy Chain (VH) Variable Domain Sequence
QVQLVQSGAEVKKPGASVKVSCKASGYSFTDYFLNWVRQAPGQGLEWMGRINPYNGDSFINQNFRDRVTMTVDTSTSTVYMELSSLRSEDTAVYYCARGGYDGYFIAYFDYWGAGTTVTVSS

SEQ ID NO: 95 - protein, 119 aa, Artificial Sequence
  Heavy Chain (VH) Variable Domain Sequence
QVQLVQSGAEVKKPGASVKVSCKASGYTFTDYEIHWVRQAPGQGLEWMGDIHPGSGGTANNQKFKGRVTMTRDTSTSTVYMELSSLRSEDTAVYYCARAVSGYYAMDYWGQGTLVTVSS

SEQ ID NO: 96 - protein, 119 aa, Artificial Sequence
  Heavy Chain (VH) Variable Domain Sequence
QVQLVQSGAEVKKPGSSVKVSCKASGYTFTDYEIHWVRQAPGQGLEWMGDIHPGSGGTANNQKFKGRVTITADESTSTAYMELSSLRSEDTAVYYCARAVSGYYAMDYWGQGTLVTVSS

SEQ ID NO: 97 - protein, 119 aa, Artificial Sequence
  Heavy Chain (VH) Variable Domain Sequence
QVQLVQSGAEVKKPGASVKVSCKASGYTFTDYEIHWVRQAPGQGLEWMGDIHPGSGGTANNQKFKGRVTMTADTSTSTVYMELSSLRSEDTAVYYCTRAVSGYYAMDYWGQGTLVTVSS

SEQ ID NO: 98 - protein, 213 aa, Artificial Sequence
  Light Chain (LC) Sequence
ENVLTQSPAIMSASLGEKVTMSCRASSGVNYMYWYQQKSDASPKLLIYYTSILAPGVPARFSGSGSGNSYSLTISSMEGEDAATYYCQQFTSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 99 - protein, 214 aa, Artificial Sequence
  Light Chain (LC) Sequence
DILLTQSPAILSVSPGERVSFSCRASQSIGTSIHWYQQRTNGSPRLLIKYGSESISGIPSRFSGSGSGTDFTLSINSVESEDIADYYCQQSNTWPLTFGDGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 100 - protein, 215 aa, Artificial Sequence
  Light Chain (LC) Sequence
EIVLTQSPTTMAASPGEKITIICSASSIIGSDFLHWYQQRPGFSPKFLIYRTSILASGVPTRFTGSGSGTSYSLTIGTMEAEDVATYYCQQGSGLPLTFGSGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 101 - protein, 214 aa, Artificial Sequence
  Light Chain (LC) Sequence
DIQMTQSPSSLSASVGDRVTITCKASQDINSHLSWYQQKPGKAPKLLIYRANRLATGVPSRFSGSGSGTDFTFTISSLQPEDIATYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 214 aa, Artificial Sequence
  Light Chain (LC) Sequence
DIQMTQSPSSLSASVGDRVTITCKASQDINSHLSWYQQKPGKAPKLLIYRANRLATGVPSRFSGSGSGTDFTFTISSLEYEDIATYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 214 aa, Artificial Sequence
  Light Chain (LC) Sequence
DIQMTQSPSSLSASVGDRVTITCKASQDINSHLSWYQQKPGKAPKLLIYRANRLATGVPSRFSGSGSGTDFTFTISSLQPEDIATYYCQQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 214 aa, Artificial Sequence
  Light Chain (LC) Sequence
DIKMTQSPSSMYASLGQRVTITCKASQDINSHLSWFQEKPGKSPKTLIYRANRLADGVPSRFSGSGSGQDYFLTISSLEYEDVGIYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 214 aa, Artificial Sequence
  Light Chain (LC) Sequence
DIQMTQSPSSLSASVGDRVTITCKASEDIYDRVAWYQQKPGKAPKLLIYGTASLETGVPSRFSGSGSGTDFTFTISSLQPEDIATYYCQQYWTTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 214 aa, Artificial Sequence
  Light Chain (LC) Sequence
DIQMTQSPSSLSASVGDRVTITCKASEDIYDRVAWYQQKPGKAPKLLIYGTASLETGVPSRFSGSGSGTDFTLTISSLQPEDFATYYCQQYWTTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 214 aa, Artificial Sequence
  Light Chain (LC) Sequence
DIQMTQSPSSLSASVGDRVTITCKASEDIYDRVAWYQQKPGKAPKLLIYGTASLETGVLSRFSGSGSGTDFTLTISSLQAEDFATYYCQQYWTTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 214 aa, Artificial Sequence
  Light Chain (LC) Sequence
DIQMTQSSSSFSGSLGDRLTINCKASEDIYDRVAWYQQKPGNAPRLLISGTASLETGVLSRFSGSGSGKDYTLSINGLQAEDVATYYCQQYWTTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 445 aa, Artificial Sequence
  Heavy Chain (HC) Sequence
QVQLQQSGAELMKPGASVKISCKATGYSFTKYWIEWVKQRPGHGLEWIGEILPGSVITNYNEKFKGKATFTADTSSNTVYMQLSSLTSEDSAVYYCTKWGLYDSDDGVDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 110 - protein, 443 aa, Artificial Sequence
  Heavy Chain (HC) Sequence
EVKLVESGGGLVQPGGSLKLSCAASGFSFSGCTMSWIRQTPERRLEWVAYISNGGDITYYPDTVKGRFTISRDNAKNSLYLQMSSLKSEDTAMYYCARLDGYYYAMDFWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 111 - protein, 445 aa, Artificial Sequence
  Heavy Chain (HC) Sequence
EVQLQQSGPEVVKPGASVKMSCKASGYTFTSYVMHWVKQKPGQGLEWIGYINPYNDGPKYNEKFKGKATLTSDKSSSTAYMELSSLTSEDSAVYFCARWDYFNSASGFAFWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 112 - protein, 449 aa, Artificial Sequence
  Heavy Chain (HC) Sequence
QVQLVQSGAEVKKPGASVKVSCKASGYSFTDYFLNWVRQAPGQGLEWMGRINPYNGDSFINQNFRDRVTMTRDTSTSTVYMELSSLRSEDTAVYYCARGGYDGYFIAYFDYWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 449 aa, Artificial Sequence
  Heavy Chain (HC) Sequence
QVQLVQSGAEVKKPGSSVKVSCKASGYSFTDYFLNWVRQAPGQGLEWMGRINPYNGDSFINQNFRDRVTITADKSTSTAYMELSSLRSEDTAVYYCARGGYDGYFIAYFDYWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 449 aa, Artificial Sequence
  Heavy Chain (HC) Sequence
EVQLVQSGAEVKKPGESLKISCKGSGYSFTDYFLNWVRQMPGKGLEWMGRINPYNGDSFINQNFRDQVTISADKSISTAYLQWSSLKASDTAMYYCARGGYDGYFIAYFDYWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 449 aa, Artificial Sequence
  Heavy Chain (HC) Sequence
QVQLVQSGAEVKKPGASVKVSCKASGYSFTDYFLNWVRQAPGQGLEWMGRINPYNGDSFINQNFRDRVTMTVDTSTSTVYMELSSLRSEDTAVYYCARGGYDGYFIAYFDYWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 449 aa, Artificial Sequence
  Heavy Chain (HC) Sequence
EVQLQQSGPDLVKPGASVKISCKASGYSFTDYFLNWVKQSHGKSLEWIGRINPYNGDSFINQNFRDKATLTVDKSSTTAHMDLLSLTSEDSAIYYCGRGGYDGYFIAYFDYWGQGSLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 446 aa, Artificial Sequence
  Heavy Chain (HC) Sequence
QVQLVQSGAEVKKPGASVKVSCKASGYTFTDYEIHWVRQAPGQGLEWMGDIHPGSGGTANNQKFKGRVTMTRDTSTSTVYMELSSLRSEDTAVYYCARAVSGYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 446 aa, Artificial Sequence
  Heavy Chain (HC) Sequence
QVQLVQSGAEVKKPGSSVKVSCKASGYTFTDYEIHWVRQAPGQGLEWMGDIHPGSGGTANNQKFKGRVTITADESTSTAYMELSSLRSEDTAVYYCARAVSGYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 446 aa, Artificial Sequence
  Heavy Chain (HC) Sequence
QVQLVQSGAEVKKPGASVKVSCKASGYTFTDYEIHWVRQAPGQGLEWMGDIHPGSGGTANNQKFKGRVTMTADTSTSTVYMELSSLRSEDTAVYYCTRAVSGYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 446 aa, Artificial Sequence
  Heavy Chain (HC) Sequence
EVQLQESGAELVRPGASVKLSCKALGYTFTDYEIHWVKETPVYGLEWIGDIHPGSGGTANNQKFKGKATLTADKSSNTAYMELSSLTSEDSAVYYCTRAVSGYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 121 - protein, 473 aa, Artificial Sequence
  SIRP alpha sequence
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 122 - protein, 359 aa, Artificial Sequence
  SIRP gamma sequence
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 123 - protein, 330 aa, Artificial Sequence [sequence omitted]

SEQ ID NO: 124 - protein, 330 aa, Artificial Sequence
  Human Fc IgG1-N297Q Sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 326 aa, Artificial Sequence
  Human Fc-IgG2 Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 126 - protein, 377 aa, Artificial Sequence
  Human Fc-IgG3 Sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 127 - protein, 326 aa, Artificial Sequence
  Human Fc-IgG4 Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 128 - protein, 326 aa, Artificial Sequence
  Human Fc-IgG4 S228P Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 129 - protein, 327 aa, Artificial Sequence
  Human Fc-IgG4 PE Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 326 aa, Artificial Sequence
  Human Fc-IgG4 PE′ Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 131 - protein, 107 aa, Artificial Sequence [sequence omitted]

(19) United States
(12) Patent Application Publication
(10) Pub. No.: US 2023/0279108 A1
(43) Pub. Date: Sep. 7, 2023

(54) THERAPEUTIC SIRP-ALPHA ANTIBODIES

(71) Applicant: Arch Oncology, Inc., Brisbane, CA (US)

(72) Inventors: Robyn PURO, St. Louis, MO (US); Ronald HIEBSCH, St. Louis, MO (US); Benjamin CAPOCCIA, Webster Groves, MO (US); Gabriela ANDREJEVA, St. Louis, MO (US); Daniel S. PEREIRA, San Diego, CA (US)

(21) Appl. No.: 17/997,524

(22) PCT Filed: Apr. 30, 2021
(86) PCT No.: PCT/US21/30163

§ 371 Date: Oct. 28, 2022

Related U.S. Application Data
(60) Provisional application No. 63/018,177, filed on Apr. 30, 2020.
(60) Provisional application No. 63/071,732, filed on Aug. 28, 2020.
(60) Provisional application No. 63/107,200, filed on Oct. 29, 2020.

(51) Int. Cl. C07K 16/28; A61P 35/00

(52) U.S. Cl. CPC C07K 16/2803; C07K 16/2863; A61P 35/00; C07K 16/2827; C07K 16/2887; C07K 2317/76; C07K 2317/92

(57) ABSTRACT

Anti-SIRPα monoclonal antibodies (anti-SIRP-alpha mAbs), including multi-specific SIRPα antibodies, are provided with distinct functional profiles as are related compositions and methods of using anti-SIRPα mAbs as therapeutics for the prevention and treatment of solid and hematological cancers. Also provided are amino acid sequences of exemplary anti-SIRPα monoclonal antibodies.

### FIELD OF THE DISCLOSURE

This disclosure pertains to the field of immunotherapy. The present disclosure provides anti-SIRPα antibodies (anti-SIRPα) which disrupt the interaction between SIRPα and CD47, enhance phagocytosis of tumor cells, cause immunomodulation of immune responses, and methods to generate anti-SIRPα antibodies and use anti-SIRPα antibodies as therapeutic agents for the prevention and treatment of hematological and solid and cancers.

### BACKGROUND

Therapeutic antibodies targeting adaptive immunity including the T-cell checkpoints, PD-1, PD-L1 and CTLA-4, to enhance the cytotoxic activity of the T-cell immune response have raised the prospect of long-term remission or even cure for patients with metastatic diseases (Hodi 2010, McDermott 2015). Despite positive results, there remains a significant patient population that either fails to respond to these checkpoint inhibitors (primary resistance) or those that respond, but eventually develop disease progression (acquired resistance) (Pitt 2016, Restifo 2016, Sharma 2017). Recent studies suggest that resistance mechanisms can be both tumor cell intrinsic, including a lack of unique tumor antigen proteins or inhibition of tumor antigen presentation, and tumor cell extrinsic, involving the absence of infiltrating T-cells, redundant inhibitory checkpoints and/or the presence of immunosuppressive cells in the tumor microenvironment (Sharma 2017). Even in tumors considered sensitive to checkpoint inhibitors, or when combining anti-CTLA-4 and anti-PD-1/PDL-1 agents, approximately 50% of patients do not experience tumor shrinkage and the median treatment duration or progression-free survival for all treated patients remains relatively short around 2-5 months (Kazandjian, 2016). In addition, several of the most prevalent solid tumors and the majority of hematological malignancies have shown disappointing results with these checkpoint inhibitors. In particular, hormone receptor-positive breast cancer, colorectal cancer (non-microsatellite instability) and prostate cancer do not appear to be sensitive to this type of immune manipulation and could benefit from a different immunotherapy approach (Le 2015, Dirix 2015, Topalian 2012, Graff 2016). These findings highlight the need for alternative or synergistic approaches that target additional checkpoints to activate the innate immune response in addition to the adaptive immune response to further improve clinical outcomes. Several checkpoints of the innate immune response are present on tumor cells and on myeloid cells (macrophages, dendritic cells, monocyte-derived suppressor cells, granulocytes) which are important cellular components of the tumor microenvironment that influence tumor progression, metastasis and overall outcome (Barclay and van den Berg 2014, Yanagita 2017).

SIgnal Regulatory Protein (SIRP)-α or SIRPα, also known as CD172a, BIT or SHPS-1, is a member of the SIRP paired receptor family of closely related SIRP proteins. SIRPα is expressed mainly by hematopoietic cells, including macrophages, dendritic cells and granulocytes, and is also expressed on neurons, especially in the brain, glia, smooth muscle cells and endothelial and some tumor cells (Barclay and van den Berg 2014). SIPRα is a transmembrane protein with an extracellular domain containing three Ig-like domains and a cytoplasmic region that contains immunoreceptor tyrosine-based inhibitory motifs (ITIMs).

The gene encoding human SIRPα is polymorphic and up to ten SIRPα haplotypes have been reported [Takenaka K. et al., 2007 and Brooke G. et al., 2004). Three allelic groups (homozygous v1/v1, heterozygous v1/v2 and homozygous v2/v2) account for virtually all ethnic groups that have been genotyped (Treffers L W et al, 2018 and Sim J. et al. 2020). A pan allele-specific antibody against SIRPα would most broadly target/block the SIRPα/CD47 checkpoint in diverse populations as in heterozygotes blocking of both alleles of SIRPα is needed to enhance macrophage mediated phagocytosis (Sim J. et al. 2020 and Zhao X W et al., 2011). The anti-SIRPα mAbs or antigen binding fragments thereof of the present disclosure, bind to both human monocytic cell lines U937 (SIRPα v1/v1) and THP-1 (SIRPα v2/v2) (Tsai et al., 2010), unlike mAb 18D5 which binds only to U937 (SIRPα v1/v1) (in cell-based binding assays) (Tsai et al., 2010 and van Eenennaam et al., 2018).

The interaction of SIRPα, expressed by myeloid cells, with CD47, expressed or overexpressed on many tumor cells as well as on some normal cells, is an important immune checkpoint of the innate response that regulates myeloid functions that include adhesion, migration, activation and inhibitory activities. The CD47/SIRPα interaction regulates macrophage and dendritic cell phagocytosis of target cells sending an inhibitory “don't eat me signal” to the phagocyte. The binding of CD47 to SIRPα initiates an inhibitory signaling cascade resulting in inhibition of phagocytosis following phosphorylation of its cytoplasmic ITIMs (Oldenborg 2000, Oldenborg 2001, Okazawa 2005), recruitment and binding of SHP-1 and SHP-2, Src homology domain-containing protein tyrosine phosphatases (Veillette 1998, Oldenborg 2001), inhibition of non-muscle myosin IIA and ultimately phagocytic function (Tsai and Discher 2008, Barclay and van den Berg 2014, Murata 2014, Veillette and Chen 2018, Matazaki 2009). An important corollary of the action of CD47 as a “don't eat me” signal is its role as a “marker of self”. This provides a significant hindrance to phagocytosis of self and blocks a subsequent autoimmune response (Oldenborg, 2002, Oldenborg 2004). Cancer cells use CD47 to mask themselves in “selfness” consequently evading both the innate and adaptive immune systems. Blocking the interaction SIRPα on innate immune cells such as macrophages and dendritic cells with CD47 on tumor cells has emerged as a viable target in cancer therapy. Preclinical data has indicated that, similar to anti-CD47 antibodies, anti-SIRPα antibodies that block the SIRPα/CD47 interaction exhibit anti-tumor efficacy in mouse tumor models, either as monotherapy or in combination with other agents (Gauttier, 2017; Ring, 2017; Yanigita, 2017; Poirier, 2018; and Guattier, 2018). Importantly, generation of an adaptive immune response, in addition to the innate immune response following interruption of the SIRPα/CD47 interaction, appears to be critical to obtaining a robust anti-tumor response (Tseng 2013, Li 2015, Xu 2017).

Expression of SIRPα on DC cells and its interaction with CD47 on T-cells appears to be important in inducing the adaptive immune response. Blockade of the SIRPα/CD47 interaction was reported to affect the DCs ability to stimulate the antigen-specific CD8+ T-cell response and this was correlated with an enhanced DC-mediated response to tumor DNA (Liu 2015, Xu 2017).

Another member of the SIRP family of paired receptors, SIRP-γ, is selectively expressed on the surface of human (but not rodent) T-cells, has a short cytoplasmic region consisting of 4 amino acids. SIRP-γ also binds to CD47 and appears to be important for mediating adhesion between T-cell and APC and for T-cell functions including proliferation and activation (Barclay and van den Berg 2014; and Piccio, 2005). Thus, blocking the interaction between SIRPα and CD47 but not between SIRP-γ and CD47 may provide an advantage to protecting T-cell function.

The present disclosure describes anti-SIRPα mAbs with distinct functional profiles. The antibodies of the disclosure are useful in various therapeutic methods for treating diseases and conditions associated with SIRPα in humans, including using anti-SIRPα mAbs as therapeutics for the prevention and treatment of solid and hematological cancers. The antibodies of the disclosure are also useful as diagnostics to determine the level of anti-SIRPα expression in tissue samples. Embodiments of the disclosure include isolated antibodies and immunologically active binding fragments thereof; pharmaceutical compositions comprising one or more of the anti-SIRPα monoclonal antibodies, preferably chimeric or humanized forms of said antibodies; and methods of therapeutic use of such anti-SIRPα monoclonal antibodies.

The embodiments of the disclosure include the mAbs, or antigen-binding fragments thereof, which are defined by reference to specific structural characteristics, i.e., specified amino acid sequences of either the CDRs or entire heavy and light-chain variable domains or entire heavy- and light-chains. All of these antibodies disclosed herein bind to either SIRPα, SIRPγ, or SIRPα and SIRPγ.

The monoclonal antibodies, or antigen binding fragments thereof may comprise at least one, usually at least three, CDR sequences as provided herein, usually in combination with framework sequences from a human variable region or as an isolated CDR peptide. In some embodiments, an antibody comprises at least one light-chain comprising the three light-chain CDR sequences provided herein situated in a variable region framework, which may be, without limitation, a murine or human variable region framework, and at least one heavy-chain comprising the three heavy-chain CDR sequences provided herein situated in a variable region framework, which may be, without limitation, a human or murine variable region framework. The monoclonal antibodies, or antigen binding fragments thereof also include single domain antibodies (e.g., nanobodies camelid VHH or shark single domain antibodies or antigen binding fragments thereof) and multi-specific (e.g., bispecific) antibodies or antigen binding fragments thereof.

In some embodiments the combinations of 6 CDRs include, but are not limited to, the combinations of variable heavy-chain CDR1 (HCDR1), variable heavy-chain CDR2 (HCDR2), variable heavy-chain CDR3 (HCDR3), variable light-chain CDR1 (LCDR1), variable light-chain CDR2 (LCDR2), and variable light-chain CDR3 (LCDR3) selected from:

HCDR1 comprising SEQ ID NO:33, HCDR2 comprising SEQ ID NO:34, HCDR3 comprising SEQ ID NO:35, LCDR1 comprising SEQ ID NO:1, LCDR2 comprising SEQ ID NO:2, LCDR3 comprising SEQ ID NO:3;

HCDR1 comprising SEQ ID NO:36, HCDR2 comprising SEQ ID NO:37, HCDR3 comprising SEQ ID NO:38, LCDR1 comprising SEQ ID NO:4, LCDR2 comprising SEQ ID NO:5, LCDR3 comprising SEQ ID NO:6;

HCDR1 comprising SEQ ID NO:39, HCDR2 comprising SEQ ID NO:40, HCDR3 comprising SEQ ID NO:41, LCDR1 comprising SEQ ID NO:7, LCDR2 comprising SEQ ID NO:8, LCDR3 comprising SEQ ID NO:9;

HCDR1 comprising SEQ ID NO:42, HCDR2 comprising SEQ ID NO:43, HCDR3 comprising SEQ ID NO:44, LCDR1 comprising SEQ ID NO:10, LCDR2 comprising SEQ ID NO:11, LCDR3 comprising SEQ ID NO:12;

HCDR1 comprising SEQ ID NO:45, HCDR2 comprising SEQ ID NO:46, HCDR3 comprising SEQ ID NO:47, LCDR1 comprising SEQ ID NO:13, LCDR2 comprising SEQ ID NO:14, LCDR3 comprising SEQ ID NO:15;

HCDR1 comprising SEQ ID NO:48, HCDR2 comprising SEQ ID NO:49, HCDR3 comprising SEQ ID NO:50, LCDR1 comprising SEQ ID NO:16, LCDR2 comprising SEQ ID NO:17, LCDR3 comprising SEQ ID NO:18;

HCDR1 comprising SEQ ID NO:51, HCDR2 comprising SEQ ID NO:52, HCDR3 comprising SEQ ID NO:53, LCDR1 comprising SEQ ID NO:19, LCDR2 comprising SEQ ID NO:20, LCDR3 comprising SEQ ID NO:21.

HCDR1 comprising SEQ ID NO:54, HCDR2 comprising SEQ ID NO:55, HCDR3 comprising SEQ ID NO:56, LCDR1 comprising SEQ ID NO:22, LCDR2 comprising SEQ ID NO:23, LCDR3 comprising SEQ ID NO:24.

HCDR1 comprising SEQ ID NO:57, HCDR2 comprising SEQ ID NO:58, HCDR3 comprising SEQ ID NO:59, LCDR1 comprising SEQ ID NO:25, LCDR2 comprising SEQ ID NO:26, LCDR3 comprising SEQ ID NO:27.

HCDR1 comprising SEQ ID NO:60, HCDR2 comprising SEQ ID NO:61, HCDR3 comprising SEQ ID NO:62, LCDR1 comprising SEQ ID NO:28, LCDR2 comprising SEQ ID NO:29, LCDR3 comprising SEQ ID NO:30.

HCDR1 comprising SEQ ID NO:42, HCDR2 comprising SEQ ID NO:43, HCDR3 comprising SEQ ID NO:44, LCDR1 comprising SEQ ID NO:10, LCDR2 comprising SEQ ID NO:31, LCDR3 comprising SEQ ID NO:12.

HCDR1 comprising SEQ ID NO:42, HCDR2 comprising SEQ ID NO:43, HCDR3 comprising SEQ ID NO:44, LCDR1 comprising SEQ ID NO:10, LCDR2 comprising SEQ ID NO:31, LCDR3 comprising SEQ ID NO:32.

HCDR1 comprising SEQ ID NO:57, HCDR2 comprising SEQ ID NO:58, HCDR3 comprising SEQ ID NO:63, LCDR1 comprising SEQ ID NO:25, LCDR2 comprising SEQ ID NO:26, LCDR3 comprising SEQ ID NO:27.

In some embodiments, the anti-SIRPα antibodies include antibodies or antigen binding fragments thereof, comprising a heavy-chain variable domain (V_{H}) having an amino acid sequence selected from the amino acid sequences of: SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97, and amino acid sequences exhibiting at least 85%, 90%, 95%, 97%, 98%, or 99% sequence identity to one of the recited sequences. Alternatively or in addition, anti-SIRPα antibodies, including antibodies or antigen binding fragments thereof, may comprise a light-chain variable domain (V_{L}) having an amino acid sequence selected from the amino acid sequences of SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, and SEQ ID NO:80, and amino acid sequences exhibiting at least 85%, 90%, 95%, 97%, 98%, or 99% sequence identity to one of the recited sequences.

Although all possible pairing of V_{H} domains and V_{L} domains selected from the V_{H} domain and V_{L} domain sequence groups listed above are permissible, certain combinations of V_{H} and V_{L} domains are disclosed. Accordingly, anti-SIRPα antibodies, or antigen binding fragments thereof, are those comprising a combination of a heavy-chain variable domain (V_{H}) and a light-chain variable domain (V_{L}), wherein the combination is selected from:
i. a heavy chain variable domain comprising the amino acid sequence of SEQ ID NO:81 and a light chain variable domain comprising the amino acid sequence SEQ ID NO:64;
ii. a heavy chain variable domain comprising the amino acid sequence of SEQ ID NO:82 and a light chain variable domain comprising the amino acid sequence SEQ ID NO:65;
iii. a heavy chain variable domain comprising the amino acid sequence of SEQ ID NO:83 and a light chain variable domain comprising the amino acid sequence SEQ ID NO:66;
iv. a heavy chain variable domain comprising the amino acid sequence of SEQ ID NO:84 and a light chain variable domain comprising the amino acid sequence SEQ ID NO:67;
v. a heavy chain variable domain comprising the amino acid sequence of SEQ ID NO:85 and a light chain variable domain comprising the amino acid sequence SEQ ID NO:68;
vi. a heavy chain variable domain comprising the amino acid sequence of SEQ ID NO:86 and a light chain variable domain comprising the amino acid sequence SEQ ID NO:69;
vii. a heavy chain variable domain comprising the amino acid sequence of SEQ ID NO:87 and a light chain variable domain comprising the amino acid sequence SEQ ID NO:70;
viii. a heavy chain variable domain comprising the amino acid sequence of SEQ ID NO:88 and a light chain variable domain comprising the amino acid sequence SEQ ID NO:71;
ix. a heavy chain variable domain comprising the amino acid sequence of SEQ ID NO:89 and a light chain variable domain comprising the amino acid sequence SEQ ID NO:72;
x. a heavy chain variable domain comprising the amino acid sequence of SEQ ID NO:90 and a light chain variable domain comprising the amino acid sequence SEQ ID NO:73;
xi. a heavy chain variable domain comprising the amino acid sequence of SEQ ID NO:91 and a light chain variable domain comprising the amino acid sequence SEQ ID NO:74;
xii. a heavy chain variable domain comprising the amino acid sequence of SEQ ID NO:91 and a light chain variable domain comprising the amino acid sequence SEQ ID NO:75;
xiii. a heavy chain variable domain comprising the amino acid sequence of SEQ ID NO:91 and a light chain variable domain comprising the amino acid sequence SEQ ID NO:76;
xiv. a heavy chain variable domain comprising the amino acid sequence of SEQ ID NO:92 and a light chain variable domain comprising the amino acid sequence SEQ ID NO:74;
xv. a heavy chain variable domain comprising the amino acid sequence of SEQ ID NO:92 and a light chain variable domain comprising the amino acid sequence SEQ ID NO:75;
xvi. a heavy chain variable domain comprising the amino acid sequence of SEQ ID NO:92 and a light chain variable domain comprising the amino acid sequence SEQ ID NO:76;
xvii. a heavy chain variable domain comprising the amino acid sequence of SEQ ID NO:93 and a light chain variable domain comprising the amino acid sequence SEQ ID NO:74;
xviii. a heavy chain variable domain comprising the amino acid sequence of SEQ ID NO:93 and a light chain variable domain comprising the amino acid sequence SEQ ID NO:75;
xix. a heavy chain variable domain comprising the amino acid sequence of SEQ ID NO:93 and a light chain variable domain comprising the amino acid sequence SEQ ID NO:76;
xx. a heavy chain variable domain comprising the amino acid sequence of SEQ ID NO:94 and a light chain variable domain comprising the amino acid sequence SEQ ID NO:74;
xxi. a heavy chain variable domain comprising the amino acid sequence of SEQ ID NO:94 and a light chain variable domain comprising the amino acid sequence SEQ ID NO:75;
xxii. a heavy chain variable domain comprising the amino acid sequence of SEQ ID NO:94 and a light chain variable domain comprising the amino acid sequence SEQ ID NO:76;
xxiii. a heavy chain variable domain comprising the amino acid sequence of SEQ ID NO:84 and a light chain variable domain comprising the amino acid sequence SEQ ID NO:77;
xxiv. a heavy chain variable domain comprising the amino acid sequence of SEQ ID NO:95 and a light chain variable domain comprising the amino acid sequence SEQ ID NO:78;
xxv. a heavy chain variable domain comprising the amino acid sequence of SEQ ID NO:95 and a light chain variable domain comprising the amino acid sequence SEQ ID NO:79;
xxvi. a heavy chain variable domain comprising the amino acid sequence of SEQ ID NO:95 and a light chain variable domain comprising the amino acid sequence SEQ ID NO:80;
xxvii. a heavy chain variable domain comprising the amino acid sequence of SEQ ID NO:96 and a light chain variable domain comprising the amino acid sequence SEQ ID NO:78;
xxviii. a heavy chain variable domain comprising the amino acid sequence of SEQ ID NO:96 and a light chain variable domain comprising the amino acid sequence SEQ ID NO:79;
xxix. a heavy chain variable domain comprising the amino acid sequence of SEQ ID NO:96 and a light chain variable domain comprising the amino acid sequence SEQ ID NO:80;
xxx. a heavy chain variable domain comprising the amino acid sequence of SEQ ID NO:97 and a light chain variable domain comprising the amino acid sequence SEQ ID NO:78;
xxxi. a heavy chain variable domain comprising the amino acid sequence of SEQ ID NO:97 and a light chain variable domain comprising the amino acid sequence SEQ ID NO:79;
xxxii. a heavy chain variable domain comprising the amino acid sequence of SEQ ID NO:97 and a light chain variable domain comprising the amino acid sequence SEQ ID NO:80; and
xxxiii. a heavy chain variable domain comprising the amino acid sequence of SEQ ID NO:89 and a light chain variable domain comprising the amino acid sequence SEQ ID NO:72.

In some embodiments, the anti-SIRPα antibodies or antigen binding fragments thereof may also comprise a combination of a heavy-chain variable domain and a light-chain variable domain wherein the heavy-chain variable domain comprises a V_{H} sequence with at least 85% sequence identity, or at least 90% sequence identity, or at least 95% sequence identity, or at least 97%, 98% or 99% sequence identity, to the heavy chain amino acid sequences shown above in (i) to (xxxiii) and/or the light chain variable domain comprises a V_{L} sequence with at least 85% sequence identity, or at least 90% sequence identity, or at least 95% sequence identity, or at least 97%, 98% or 99% sequence identity, to the light-chain amino acid sequences shown above in (i) to (xxxiii). The specific V_{H} and V_{L} pairings or combinations in parts (i) through (xxxiii) may be preserved for anti-SIRPα antibodies having V_{H} and V_{L} domain sequences with a particular percentage sequence identity to these reference sequences.

For all embodiments the heavy-chain and/or light-chain variable domains of the antibodies or antigen binding fragments are defined by a particular percentage sequence identity to a reference sequence, the V_{H} and/or V_{L} domains may retain identical CDR sequences to those present in the reference sequence such that the variation is present only within the framework regions.

In another embodiment, the anti-SIRPα antibodies, or antigen binding fragments thereof, are those comprising a combination of a heavy chain (HC) and a light chain (LC), wherein the combination is selected from:
i. a heavy chain comprising the amino acid sequence of SEQ ID NO:109 and a light chain comprising the amino acid sequence SEQ ID NO:98;
ii. a heavy chain comprising the amino acid sequence of SEQ ID NO:110 and a light chain comprising the amino acid sequence SEQ ID NO:99;
iii. a heavy chain comprising the amino acid sequence of SEQ ID NO:111 and a light chain comprising the amino acid sequence SEQ ID NO:100.
iv. a heavy chain comprising the amino acid sequence of SEQ ID NO:112 and a light chain comprising the amino acid sequence SEQ ID NO:101;
v. a heavy chain comprising the amino acid sequence of SEQ ID NO:112 and a light chain comprising the amino acid sequence SEQ ID NO:102;
vi. a heavy chain comprising the amino acid sequence of SEQ ID NO:112 and a light chain comprising the amino acid sequence SEQ ID NO:103;
vii. a heavy chain comprising the amino acid sequence of SEQ ID NO:113 and a light chain comprising the amino acid sequence SEQ ID NO:101;
viii. a heavy chain comprising the amino acid sequence of SEQ ID NO:113 and a light chain comprising the amino acid sequence SEQ ID NO:102;
ix. a heavy chain comprising the amino acid sequence of SEQ ID NO:113 and a light chain comprising the amino acid sequence SEQ ID NO:103;
x. a heavy chain comprising the amino acid sequence of SEQ ID NO:114 and a light chain comprising the amino acid sequence SEQ ID NO:101;
xi. a heavy chain comprising the amino acid sequence of SEQ ID NO:114 and a light chain comprising the amino acid sequence SEQ ID NO:102;
xii. a heavy chain comprising the amino acid sequence of SEQ ID NO:114 and a light chain comprising the amino acid sequence SEQ ID NO:103;
xiii. a heavy chain comprising the amino acid sequence of SEQ ID NO:115 and a light chain comprising the amino acid sequence SEQ ID NO:101;
xiv. a heavy chain comprising the amino acid sequence of SEQ ID NO:115 and a light chain comprising the amino acid sequence SEQ ID NO:102;
xv. a heavy chain comprising the amino acid sequence of SEQ ID NO:115 and a light chain comprising the amino acid sequence SEQ ID NO:103;
xvi. a heavy chain comprising the amino acid sequence of SEQ ID NO:116 and a light chain comprising the amino acid sequence SEQ ID NO:104;
xvii. a heavy chain comprising the amino acid sequence of SEQ ID NO:117 and a light chain comprising the amino acid sequence SEQ ID NO:105;
xviii. a heavy chain comprising the amino acid sequence of SEQ ID NO:117 and a light chain comprising the amino acid sequence SEQ ID NO:106;
xix. a heavy chain comprising the amino acid sequence of SEQ ID NO:117 and a light chain comprising the amino acid sequence SEQ ID NO:107;
xx. a heavy chain comprising the amino acid sequence of SEQ ID NO:118 and a light chain comprising the amino acid sequence SEQ ID NO:105;
xxi. a heavy chain comprising the amino acid sequence of SEQ ID NO:118 and a light chain comprising the amino acid sequence SEQ ID NO:106;
xxii. a heavy chain comprising the amino acid sequence of SEQ ID NO:118 and a light chain comprising the amino acid sequence SEQ ID NO:107;
xxiii. a heavy chain comprising the amino acid sequence of SEQ ID NO:119 and a light chain comprising the amino acid sequence SEQ ID NO:105;
xxiv. a heavy chain comprising the amino acid sequence of SEQ ID NO:119 and a light chain comprising the amino acid sequence SEQ ID NO:106;
xxv. a heavy chain comprising the amino acid sequence of SEQ ID NO:119 and a light chain comprising the amino acid sequence SEQ ID NO:107; and
xxvi. a heavy chain comprising the amino acid sequence of SEQ ID NO:120 and a light chain comprising the amino acid sequence SEQ ID NO:108.

Various forms of the anti-SIRPα mAbs are disclosed. For example, the anti-CD47 mAbs can be full length humanized antibodies with human frameworks and constant regions of the isotypes, IgA, IgD, IgE, IgG, and IgM, more particularly, IgG1, IgG2, IgG3, IgG4, and in some cases with various mutations to alter Fc receptor function or prevent Fab arm exchange or an antibody fragment, e.g., a F(ab′)2 fragment, a F(ab) fragment, a single chain Fv fragment (scFv), etc., as disclosed herein.

In some embodiments, the anti-SIRPα mAbs or antigen-binding fragment thereof comprises an IgG isotype selected from IgG1, IgG1-N297Q, IgG2, IgG4, IgG4 S228P, IgG4 PE and variants thereof.

In some embodiments, the anti-SIRPα mAbs or antigen-binding fragment thereof binds human SIRPγ in addition to human SIRPα.

In some embodiments, the anti-SIRPα mAbs or antigen-binding fragment thereof selectively binds human SIRPα.

In some embodiments, the anti-SIRPα mAbs or antigen-binding fragment thereof increases phagocytosis of human tumor cells.

In some embodiments, the anti-SIRPα mAbs as disclosed herein are multi-specific antibodies that specifically bind to SIRPα and at least a second antigen, where the second antigen is a marker of a CD47-expressing cell.

In some embodiments, the second antigen of the multi-specific antibody is selected from CD19, CD20, CD22, CD24, CD25, CD30, CD33, CD40, CD44, HER2, CD52, CD56, CD70, CD96, CD97, CD99, CD123, CD279 (PD-1), CD117, C-Met, PTHR2, EGFR, RANKL, SLAMF7, PD-L1, CD38, CD19/CD3, HAVCR2 (TIM3), and GD2.

In some embodiments, the anti-SIRPα mAbs or antigen-binding fragment thereof increases phagocytosis of human tumor cells.

In some embodiments, the increased phagocytosis of human tumor cells is Fc-independent.

In some embodiments, the increased phagocytosis of human tumor cells is Fc-dependent.

In some embodiments, the increased phagocytosis of human tumor cells phagocytosis is dependent on FcγR.

In some embodiments, the FcγR is chosen from FcγRI (CD64), FcγRIIA (CD32), FcγRIIB (CD32), FcγRIIIA (CD16a), and FcγRIIIB (CD16b).

In some embodiments, the anti-SIRPα mAbs or antigen-binding fragment thereof increases phagocytosis of human tumor cells and are administered in combination with an opsonizing monoclonal antibody that targets an antigen on a tumor cell.

In some embodiments, the anti-SIRPα mAbs or antigen-binding fragment thereof increases phagocytosis of human tumor cells and are administered in combination with an opsonizing monoclonal antibody that targets an antigen on a tumor cell, wherein the opsonizing monoclonal antibody is selected from one or more of anti-CD20, anti-HER2, anti-CD52, anti-EGFR, anti-RANKL, anti-SLAMF7, anti-PD-L1, anti-CD38, anti-CD19/CD3, and anti-GD2 antibodies. In some embodiments, the opsonizing monoclonal antibody is selected from one or more of rituximab, trastuzumab, alemtuzumab, cetuximab, panitumumab, ofatumumab, denosumab, pertuzumab, panitumumab, elotuzumab, atezolizumab, avelumab, durvalumab, necitumumab, daratumumab, obinutuzumab, blinatumomab, and dinutuximab.

In some embodiments, the opsonizing monoclonal antibody targets CD20, EGFR, and PD-L1.

In some embodiments, the anti-SIRPα mAbs or antigen-binding fragment thereof exhibits anti-tumor activity.

In some embodiments, the anti-SIRPα mAbs or antigen-binding fragment thereof is administered in combination with an anti-CD47 antibody, wherein the anti-CD47 antibody is described in U.S. Pat. No. 10,239,945, and hereby incorporated by reference in its entirety.

In some embodiments, the anti-SIRPα mAbs or antigen-binding fragment thereof is administered in combination with an anti-EGFR antibody.

In some embodiments, the anti-SIRPα mAbs or antigen-binding fragment thereof is administered in combination with an anti-PD-1 antibody.

In some embodiments, the anti-SIRPα mAbs or antigen-binding fragment thereof is administered in combination with an anti-CTLA-4 antibody.

In some embodiments, the disclosure provides a pharmaceutical composition comprising one or more of the anti-SIRPα mAbs or antigen-binding fragments disclosed herein, optionally in chimeric or humanized forms, and a pharmaceutically or physiologically acceptable carrier, diluent, or excipient.

In some embodiments, the anti-SIRPα mAbs or antigen-binding fragment thereof are for use in human therapy.

In some embodiments, the anti-SIRPα mAbs or antigen-binding fragment thereof are for use in preventing or treating cancer in a human patient.

Prior to the present disclosure, there was a need to identify anti-SIRPα mAbs that possess the functional profiles as described herein. The anti-SIRPα mAbs of the present disclosure exhibit a combination of properties that render the mAbs particularly advantageous for use in human therapy, particularly in the prevention and/or treatment of solid and hematological cancers.

In some embodiments, the cancer is selected from leukemia, a lymphoma, multiple myeloma, ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, melanoma, myelodysplastic syndrome, and a sarcoma.

In some embodiments, the leukemia is selected from leukemia is selected from the group consisting of systemic mastocytosis, acute lymphocytic (lymphoblastic) leukemia (ALL), T-cell—ALL, acute myeloid leukemia (AML), myelogenous leukemia, chronic lymphocytic leukemia (CLL), chronic myeloid leukemia (CML), myeloproliferative disorder/neoplasm, myelodysplastic syndrome, monocytic cell leukemia, and plasma cell leukemia; wherein said lymphoma is selected from the group consisting of histiocytic lymphoma and T-cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia; and wherein said sarcoma is selected from the group consisting of osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma.

In some embodiments, a method is disclosed to assay SIRPα expression in tumor and/or immune cells using an anti-SIRPα monoclonal antibody or antigen-binding fragment thereof, which specifically binds to an epitope within the sequence of SEQ ID NO:121.

In some embodiments, the method comprises obtaining a patient sample, contacting the patient sample with an anti-SIRPα monoclonal antibody or antigen-binding fragment thereof, which specifically binds to an epitope within the sequence of SEQ ID NO:121, and assaying for binding of the antibody to the patient sample, wherein binding of the antibody to the patient sample is diagnostic of SIRPα expression in a patient sample.

In some embodiments, a method is disclosed to assay SIRPγ expression in tumor and or immune cells using an anti-SIRPα monoclonal antibody or antigen-binding fragment thereof, which specifically binds to an epitope within the sequence of SEQ ID NO:122.

In some embodiments, the method comprises obtaining a patient sample, contacting the patient sample with an anti-SIRPγ monoclonal antibody or antigen-binding fragment thereof, which specifically binds to an epitope within the sequence of SEQ ID NO:122, and assaying for binding of the antibody to the patient sample, wherein binding of the antibody to the patient sample is diagnostic of SIRPγ expression in a patient sample.

In some embodiments, the tumor is primary a cancer tumor or a metastatic cancer tumor.

In some embodiments, assaying for binding of the anti-SIRPα monoclonal antibody or antigen-binding fragment thereof to the patient sample utilizes immunohistochemistry labeling of a tissue sample, enzyme linked immunosorbent assay (ELISA), or flow cytometry.

In some embodiments, the method comprises tumor cells, and the assay comprises assaying for the binding of the anti-SIRPα monoclonal antibody or antigen-binding fragment thereof to tumor cells in the patient sample.

Further scope of the applicability of the present disclosure will become apparent from the detailed description provided below. However, it should be understood that the detailed description and specific examples, while indicating embodiments of the disclosure, are given by way of illustration only since various changes and modifications within the spirit and scope of the disclosure will become apparent to those skilled in the art from this detailed description.

### BRIEF DESCRIPTION OF THE DRAWINGS

The above and other aspects, features, and advantages of the present disclosure will be better understood from the following detailed descriptions taken in conjunction with the accompanying drawing(s), all of which are given by way of illustration only and are not limited in the present disclosure.

FIG. 1A-FIG. 1V. Binding of anti-SIRP antibodies to human SIRPα. Binding of anti-SIRP antibodies to recombinant human SIRPα was determined by solid-phase ELISA. High-binding ELISA plates were coated with recombinant human SIRPα and increasing concentrations of anti-SIRP antibodies were added for 1 hour. Wells were washed and then incubated with HRP-labeled secondary antibody for 1 hour followed by addition of peroxidase substrate and the absorbance at 450 nm was measured. Results are shown for anti-SIRPα antibodies 1-23.

FIG. 2. Binding of Hybridoma Derived mAbs (SIRP1, SIRP2, and SIRP3) to THP1 cells Expressing SIRPα. Binding of SIRP1, SIRP2, and SIRP3 to THP-1 monocytic cell line was determined. Cells were incubated with increasing concentrations of antibody for 1 hr. Cells were washed and then incubated with Alexaflour 647-labelled secondary antibody for 1 hr. Cells were washed and antibody binding measured using flow cytometry.

FIG. 3A-FIG. 3V. Binding of anti-SIRP antibodies to human SIRP gamma. Binding of anti-SIRP antibodies to recombinant human SIRP gamma (SIRPγ) was determined by solid-phase ELISA. High-binding ELISA plates were coated with recombinant human SIRP gamma and increasing concentrations of anti-SIRP antibodies were added for 1 hour. Wells were washed and then incubated with HRP-labeled secondary antibody for 1 hour followed by addition of peroxidase substrate and the absorbance at 450 nm was measured. Results are shown for anti-SIRPα antibodies 1-23.

FIG. 4A-FIG. 4B. Binding of SIRP mAbs to Jurkat T cells Expressing SIRPγ. Binding of SIRP1, SIRP2, SIRP3, SIRP4 and SIRP9 to Jurkat T-ALL cells was determined. Cells were incubated with increasing concentrations of antibody FIG. 4A; or 10 μg/ml of the anti-SIRP antibodies for 1 hr; FIG. 4B. Cells were washed and then incubated with Alexaflour 647-labelled secondary antibody for 1 hr. Cells were washed and antibody binding measured using flow cytometry.

FIG. 5A-FIG. 5G. Blocking of human CD47/SIRPα binding by anti-SIRP antibodies. The ability of anti-SIRP antibodies to block the interaction between CD47 and recombinant human SIRα was determined by solid-phase ELISA. High-binding ELISA plates were coated with recombinant human SIRPα and increasing concentrations of anti-SIRP antibodies were added for 1 hour. Wells were washed and then incubated with an Fc tagged human CD47 for 1 hours. Wells were washed and then incubated with an HRP-labeled secondary antibody for 1 hour followed by addition of peroxidase substrate and the absorbance at 450 nm was measured.

FIG. 6A-FIG. 6H. Blocking of human CD47/SIRPγ binding by anti-SIRP antibodies. The ability of anti-SIRP antibodies to block the interaction between CD47 and recombinant human SIRPγ was determined by solid-phase ELISA. High-binding ELISA plates were coated with recombinant human SIRPγ and increasing concentrations of anti-SIRP antibodies were added for 1 hour. Wells were washed and then incubated with an Fc tagged human CD47 for 1 hours. Wells were washed and then incubated with an HRP-labeled secondary antibody for 1 hour followed by addition of peroxidase substrate and the absorbance at 450 nm was measured.

FIG. 7A-FIG. 7B. Anti-SIRP antibodies enhance phagocytosis. Human macrophages were plated at a concentration of 3×10⁴ cells per well in a 96 well plate and allowed to adhere for 24 hours. 8×10⁴ CFSE (1 μM) labeled human Jurkat T cells and increasing concentrations of anti-SIRP antibodies; FIG. 7A or 10 μg/ml of the anti-SIRP antibodies, FIG. 7B, were added to the macrophage cultures and incubated at 37° C. for 3 hours. Non-phagocytosed Jurkat cells were removed and macrophage cultures were washed. Macrophages were trypsinized and stained for CD14. Flow cytometry was used to determine the percentage of CD14⁺/CFSE⁺ cells in the total CD14⁺ population.

FIG. 8A-FIG. 8J. Anti-SIRP antibodies enhance phagocytosis in combination with anti-CD47 antibodies. Human macrophages were plated at a concentration of 3×10⁴ cells per well in a 96 well plate and allowed to adhere for 24 hours. 8×10⁴ CFSE (1 μM) labeled human Jurkat T cells and increasing concentrations of anti-SIRP antibodies alone, anti-CD47 antibody alone, or a combination of anti-SIRP antibodies and anti-CD47 antibody were added to the macrophage cultures and incubated at 37° C. for 3 hours. Non-phagocytosed Jurkat cells were removed and macrophage cultures were washed. Macrophages were trypsinized and stained for CD14. Flow cytometry was used to determine the percentage of CD14⁺/CFSE⁺ cells in the total CD14⁺ population.

FIG. 9A-FIG. 9D. Anti-SIRP antibodies enhance phagocytosis in combination with anti-CD20 antibodies. Human macrophages were plated at a concentration of 3×10⁴ cells per well in a 96 well plate and allowed to adhere for 24 hours. 8×10⁴ CFSE (1 μM) labeled human RAJI lymphoma cells and increasing concentrations of anti-SIRP antibodies alone, the anti-CD20 antibody Rituxan alone, or a combination of anti-SIRP antibodies and Rituxan were added to the macrophage cultures and incubated at 37° C. for 3 hours. Non-phagocytosed RAJI cells were removed and macrophage cultures were washed. Macrophages were trypsinized and stained for CD14. Flow cytometry was used to determine the percentage of CD14⁺/CFSE⁺ cells in the total CD14⁺ population.

FIG. 10A-FIG. 10B. Anti-SIRP antibodies enhance phagocytosis in combination with anti-EGFR and anti-PD-L1 antibodies. Human macrophages were plated at a concentration of 3×10⁴ cells per well in a 96 well plate and allowed to adhere for 24 hours. 8×10⁴ CFSE (1 μM) labeled human FaDu HNSCC and increasing concentrations of anti-SIRP antibodies alone, the anti-EGFR antibody Erbitux alone, or anti-SIRP antibodies in combination with Erbitux or in combination with Avelumab were added to the macrophage cultures and incubated at 37° C. for 3 hours. Non-phagocytosed FaDu cells were removed and macrophage cultures were washed. Macrophages were trypsinized and stained for CD14. Flow cytometry was used to determine the percentage of CD14⁺/CFSE⁺ cells in the total CD14⁺ population.

FIG. 11. Anti-SIRP antibodies bind to SIRPα on macrophages and dendritic cells. Binding of anti-SIRP antibodies to human macrophages or dendritic cells was determined. Human monocyte-derived macrophages were incubated with increasing concentrations of anti-SIRP antibodies for 1 hr. The cells were washed and then incubated with AF647-labelled secondary antibody for 45 min, washed and antibody binding measured using flow cytometry.

FIG. 12A-FIG. 12C. Anti-SIRP antibodies bind to SIRPγ on naïve and activated T cells. Binding of anti-SIRP antibodies to naïve T cells (FIG. 12A and FIG. 12B) or activated T cells (FIG. 12C) following 3-day activation on anti-CD3 coated plates was determined by flow cytometry. T cells were incubated with increasing concentrations of anti-SIRP antibodies for 1 h, cells were washed and FITC-labelled anti-mouse secondary antibody was added for 1 hr. Cells were washed and antibody binding measured using flow cytometry.

FIG. 13. Blocking of human CD47/SIRPα binding by anti-SIRP antibodies on macrophages. The ability of anti-SIRP antibodies to block the interaction between recombinant human CD47 and macrophage expressed SIRPα was determined by flow cytometry. The Fc receptors on macrophages were blocked prior to incubation with 10 μg/ml of the anti-SIRP antibodies. Binding of soluble Fc tagged human CD47 (20 μg/ml) was measured using AF647-tagged anti-human secondary antibody.

FIG. 14A-FIG. 14B. Anti-SIRP antibodies do not inhibit T cell proliferation upon allogeneic dendritic cell stimulation. Effect of anti-SIRP antibodies on proliferation of T cells was determined by activating CellTrace Violet labelled human CD3 T cells with allogeneic human monocyte-derived dendritic cells at a 1:5 T cell:DC ratio in the presence of 10 μg/ml anti-SIRP antibodies. Flow cytometry was used to determine the percentage of proliferated CD3 T cells following 6-7-day co-culture. The dotted line represents proliferation of hIgG4P control.

FIG. 15. Anti-SIRP antibodies do not inhibit antigen recall response. Effect of anti-SIRP antibodies on T cell antigen recall responses was assessed using PBMC from human cytomegalovirus seropositive donor. CellTrace Violet dye-labelled PBMC were incubated with 10 μg/ml of anti-SIRP antibodies in the presence of increasing concentrations of CMV antigen for 5 days. T cell proliferation was determined by the dilution of the CellTrace Violet dye within the CD4+ T cell population using flow cytometry.

FIG. 16. Anti-SIRP antibodies effect SIRPα higher order structures and/or architecture. Effect of anti-SIRP antibodies on the dimerization/clustering/architecture of SIRPα was assessed by measuring fluorescence resonance energy transfer (FRET) efficiency between phycoerythrin (PE) and allophycocyanin (APC) conjugated non-competing anti-SIRP antibodies using flow cytometry (FCET). Human CD14+ monocytes were cultured in AIM-V medium supplemented with 50 ng/ml M-CSF for 6 days to differentiate them into macrophages. Following treatment with 10 μg/ml anti-SIRP antibodies for 3 h, the macrophages were stained with non-competing SIRPα antibodies (clone SE5A5) labeled with PE or APC. FRET efficiency between PE/APC pair was assayed using flow cytometry and three-wavelength correction model was used.

FIG. 17. Anti-SIRP antibodies induce internalization of SIRPα-antibody. Effect of anti-SIRP antibodies was assessed on internalization of SIRPα-antibody complexes using human macrophages and pHrodo green-labeled anti-SIRP antibodies. For macrophage differentiation, human CD14+ monocytes, isolated from peripheral blood mononuclear cells, were cultured in vitro for seven days in AIM-V medium supplemented with 50 ng/ml M-CSF. The macrophages were incubated with 10 μg/ml anti-SIRP antibodies at 37° C. for up to 1 h and internalization measured by flow cytometry. SIRP antibodies were labelled using pHrodo Green Microscale Protein Labeling Kit.

FIG. 18. Anti-SIRP Monoclonal Antibodies Reduce Cell Surface SIRPα Levels. Effect of anti-SIRP antibodies was assessed on the expression level of SIRPα on the cell surface by flow cytometry using human macrophages. Human CD14+ monocytes, isolated from peripheral blood mononuclear cells, were differentiated in vitro for seven days in AIM-V medium supplemented with 50 ng/ml M-CSF to produce human macrophages. Human macrophages were incubated in AIM-V medium for 2 h in V-bottom 96-well plates. The cells were then incubated with 10 μg/ml mIgG1 control, SIPR4, SIRP9, 18D5 or KWAR23 for 2 h at 4° C. or for 2 h, 4 h, 6 h or 24 h at 37° C., 5% CO₂. Subsequently, the cell surface SIRPα level measured using non-competing fluorescently labelled anti-SIRPα antibodies by flow cytometry.

FIG. 19A-FIG. 19B. Anti-SIRP antibodies induce phagocytosis of tumor cells independent of SIRP alpha allelic variant. Effect of anti-SIRPα antibodies on phagocytosis of tumor cells by macrophages bearing different SIRPα allelic variants was assessed by an in vitro assay using flow cytometry. Human monocyte derived macrophages from different SIRPα allelic group donors (V1/V1, V1/V2, and V2/V2) were differentiated from CD14+ monocytes and cultured in AIM-V medium without supplements for 2 h. Human cancer cells were labeled with 1 μM 5(6)-Carboxyfluorescein diacetate N-succinimidyl ester and added to the macrophage cultures in 96-well plates at a concentration of 8×10⁴ cells/well. Anti-SIRP antibodies were added immediately upon mixture of target and effector cells. FIG. 19A shows anti-SIRP or mIgG1 control antibodies added at various concentrations and FIG. 19B shows concentrations of 10 μg/ml. After 4 h incubation at 37° C., all non-phagocytosed cells were removed, and the remaining cells washed three times with PBS. Macrophages were detached using Accutase and stained with 100 ng of allophycocyanin (APC) labeled CD14 antibodies for 30 minutes, and analyzed by flow cytometry. Phagocytosis was determined as the percentage of CFSE+ cells within the CD14+ cell population.

FIG. 20. Anti-SIRP Monoclonal Antibodies do not Compete with each other for SIRPα Binding. The competition between anti-SIRP antibodies in binding to human SIRPα was assessed by ELISA using His-tagged human SIRPα protein and biotinylated SIRPα antibodies.

FIG. 21A-FIG. 21B. Anti-SIRP antibodies binding to human cancer cell lines correlates to their expression of SIRPα and SIRPγ. The binding of anti-SIRP mAbs and surface expression of SIRPα/β, SIRPγ, and CD47 in human cancer cell lines (Jurkat T-ALL, RAJI B cell lymphoma, DLD-1 colorectal adenocarcinoma, RL95-2 endometrial carcinoma, and ES-2 ovarian carcinoma) used in phagocytosis assays was assessed by flow cytometry. FIG. 21A shows expression of CD47 as measured by commercial anti-CD47 clone B6H12, SIRPα/β (measured by commercial clone SE5A5), and SIRPγ (measured by commercial clone LSB2.20) on cancer cell lines used in phagocytosis assays. FIG. 21B shows the binding of SIRP4 and SIRP9 correlates well with SIRPα/β or SIRPγ expression seen by commercial anti-SIRP antibodies. **** indicates p<0.0001.

FIG. 22A-FIG. 22D. Anti-SIRP antibody induced phagocytosis involves Fc receptors. Human monocyte derived macrophages were plated at a concentration of 3×10⁴ cells per well in a 96 well plate and allowed to adhere for 24 hours. 10 μg/ml antibodies against human Fc receptors CD16, CD32 and CD64 (+Fc block) or 30 μg/ml mIgG1 isotype control (−Fc block) were then added. Immediately afterwards, 8×10⁴ CFSE (1 μM) labeled human Jurkat T-ALL (FIG. 22A-FIG. 22B) cells or DLD-1 cells (FIG. 22C-FIG. 22D) and increasing concentrations of anti-SIRP antibodies SIRP4 (FIG. 22A-FIG. 22C) or SIRP9 (FIG. 22B-FIG. 22D) were added to the macrophage cultures and incubated at 37° C. for 3 hours. Non-phagocytosed cancer cells were removed and macrophage cultures were washed. Macrophages were trypsinized and stained for CD14. Flow cytometry was used to determine the percentage of CD14⁺/CFSE⁺ cells in the total CD14⁺ population.

FIG. 23A-FIG. 23B. Anti-SIRP antibody induced phagocytosis depends on FcγRII. Human monocyte derived macrophages were plated at a concentration of 3×10⁴ cells per well in a 96 well plate and allowed to adhere for 24 hours. 10 μg/ml antibodies against human Fc receptors CD16, CD32 and CD64 or mIgG1 isotype control were then individually added. Immediately afterwards, 8×10⁴ CFSE (I1 μM) labeled human Jurkat T-ALL cells and increasing concentrations of anti-SIRP antibodies SIRP4 (FIG. 23A) or SIRP9 (FIG. 23B) were added to the macrophage cultures and incubated at 37° C. for 3 hours. Non-phagocytosed cancer cells were removed, and macrophage cultures were washed. Macrophages were trypsinized and stained for CD14. Flow cytometry was used to determine the percentage of CD14⁺/CFSE⁺ cells in the total CD14⁺ population.

FIG. 24A-FIG. 24B. Anti-SIRP antibodies do not induce phagocytosis of normal autologous human peripheral blood mononuclear cells (PBMCs). Human monocyte derived macrophages were plated at a concentration of 3×10⁴ cells per well in a 96 well plate and allowed to adhere for 24 hours. 8×10⁴ CFSE (1 μM) labeled autologous normal PBMCs (FIG. 24A) or human Jurkat T-ALL cells (FIG. 24B) and increasing concentrations of anti-SIRP antibodies SIRP4 or SIRP9 were added to the macrophage cultures and incubated at 37° C. for 3 hours. Non-phagocytosed cells were removed, and macrophage cultures were washed. Macrophages were trypsinized and stained for CD14. Flow cytometry was used to determine the percentage of CD14⁺/CFSE⁺ cells in the total CD14⁺ population.

### DETAILED DESCRIPTION OF THE DISCLOSURE

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

As used herein, the term “SIRPα” and “Src homology 2 (SH2) domain-containing protein tyrosine phosphatase substrate 1 (SHPS-1)” are synonymous and may be used interchangeably.

The term “anti-SIRPα antibody” refer to an antibody of the disclosure which is intended for use as a therapeutic or diagnostic agent, and specifically binds to SIRPα, in particular to a human SIRPα.

The term “anti-SIRP” refer to an antibody of the disclosure which is intended for use as a therapeutic or diagnostic agent, and specifically binds to SIRPα, in particular to a human SIRPα, to one or both of two common variants identified, SIRPαV1 and SIRPαV2, and/or SIRPγ and antibody variants thereof.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts” with or directed against is meant that the antibody reacts with one or more antigenic determinants of the desired antigen and does not react with other polypeptides or binds at a much lower affinity (K_{d}>10⁻⁶ M). Antibodies include but are not limited to, polyclonal, monoclonal, chimeric, Fab fragments, Fab′ fragments, F(ab′)₂ fragments, single chain Fv fragments, and one-armed antibodies.

As used herein, the term “monoclonal antibody (mAb)” as applied to the present anti-SIRPα compounds refer to an antibody that is derived from a single copy or clone including, for example, any eukaryotic, prokaryotic, or phage clone, and not the method by which it is produced. Monoclonal antibodies of the present disclosure preferably exist in a homogeneous or substantially homogeneous population. Complete mAbs contain 2 heavy-chains and 2 light-chains.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments.

As disclosed herein, “antibody compounds” refers to mAbs and antigen-binding fragments thereof. Additional antibody compounds exhibiting similar functional properties according to the present disclosure can be generated by conventional methods. For example, mice can be immunized with human SIRPα or fragments thereof, the resulting antibodies can be recovered and purified, and determination of whether they possess binding and functional properties similar to or the same as the antibody compounds disclosed herein can be assessed by the methods disclosed in the Examples. Antigen-binding fragments can also be prepared by conventional methods. Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

As disclosed herein, “multi-specific antibodies” are e.g., bispecific, tri-specific or tetraspecific antibodies. In some embodiments, the multi-specific antibodies target SIRPα and/or SIRPγ and at least one other antigen binding specificity in one molecule. In some embodiments, the multi-specific antibodies may simultaneously target SIRPα and/or SIRPγ and at least a second antigen (bispecific), or at least a second and third antigen (tri-specific), or at least a second, third, and fourth antigen (tetra-specific), wherein the second antigen, third antigen, and fourth antigen is on a tumor cell as disclosed herein.

Bispecific antibodies are antibodies which have two different antigen binding specificities in one molecule. Tri-specific antibodies, accordingly, are antibodies which have three different antigen-binding specificities in one molecule. Tetra-specific antibodies are antibodies which have four different antigen-binding specificities in one molecule. In one embodiment, the anti-SIRPα antibodies as disclosed herein are bispecific antibodies targeting SIRPα and/or SIRPγ, and a second antigen on a tumor cell as disclosed herein.

In some embodiments, bispecific antibodies may include molecular formats with symmetric or asymmetric architecture.

Asymmetric bispecific antibodies may include but are not limited to bispecific antibody conjugates, hybrid bispecific IgGs, hybrid bispecific IgMs, “variable domain only” bispecific antibody molecules, C_{H1}/C_{L} fusion proteins, Fab fusion proteins, non-immunoglobulin fusion proteins, Fc-modified IgGs, Fc-modified IgMs, appended and Fc-modified IgGs, appended and Fc-modified IgGM, and modified Fc and C_{H3} fusion proteins.

Symmetric bispecific antibodies may include but are not limited to appended IgGs—HC fusions, appended IgGs—LC fusions, appended IgGs—HC and LC fusions, Fc fusions, C_{H3} fusions, IgE, IgM C_{H2} fusions, F(ab′)₂, C_{H1}/CL fusion proteins, modified IgGs, and non-immunoglobulin fusions.

The monoclonal antibodies encompass antibodies in which a portion of the heavy and/or light-chain is identical with, or homologous to, corresponding sequences in murine antibodies, in particular the murine CDRs, while the remainder of the chain(s) is (are) identical with, or homologous to, corresponding sequences in human antibodies. Other embodiments of the disclosure include antigen-binding fragments of these monoclonal antibodies that exhibit binding and biological properties similar or identical to the monoclonal antibodies. The antibodies of the present disclosure can comprise kappa or lambda light-chain constant regions, and heavy-chain IgA, IgD, IgE, IgG, or IgM constant regions, including those of IgG subclasses IgG1, IgG2, IgG3, and IgG4 and in some cases with various mutations to alter Fc receptor function.

The monoclonal antibodies containing the presently disclosed murine CDRs can be prepared by any of the various methods known to those skilled in the art, including recombinant DNA methods.

Reviews of current methods for antibody engineering and improvement can be found, for example, in P. Chames, Ed., (2012) Antibody Engineering: Methods and Protocols, Second Edition (Methods in Molecular Biology, Book 907), Humana Press, ISBN-10: 1617799734; C. R. Wood, Ed., (2011) Antibody Drug Discovery (Molecular Medicine and Medicinal Chemistry, Book 4), Imperial College Press; R. Kontermann and S. Dubel, Eds., (2010) Antibody Engineering Volumes 1 and 2 (Springer Protocols), Second Edition; and W. Strohl and L. Strohl (2012) Therapeutic antibody engineering: Current and future advances driving the strongest growth area in the pharmaceutical industry, Woodhead Publishing.

Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

A full-length antibody as it exists naturally is a “Y” shaped immunoglobulin (Ig) molecule comprising four polypeptide chains: two identical heavy (H) chains and two identical light (L) chains, interconnected by disulfide bonds. The amino terminal portion of each chain, termed the fragment antigen binding region (FAB), includes a variable region of about 100-110 or more amino acids primarily responsible for antigen recognition via the complementarity determining regions (CDRs) contained therein. The carboxy-terminal portion of each chain defines a constant region (the “Fc” region) primarily responsible for effector function.

The CDRs are interspersed with regions that are more conserved, termed frameworks (“FRs”). Amino acid sequences of many FRs are well known in the art. Each light-chain variable region (LCVR) and heavy-chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light-chain are referred to as “LCDR1, LCDR2, and LCDR3” and the 3 CDRs of the heavy-chain are referred to as “HCDR1, HCDR2, and HCDR3.” The CDRs contain most of the residues which form specific interactions with the antigen. The numbering and positioning of CDR amino acid residues within the LCVR and HCVR regions are in accordance with the well-known Kabat numbering convention Kabat et al. (1991) Sequences of Proteins of Immunological Interest, Fifth Edition. NIH Publication No. 91-3242.

As described herein, the “antigen-binding site” can also be defined as the “Hypervariable regions”, “HVRs”, or “HVs”, and refer to the structurally hypervariable regions of antibody variable domains as defined by Chothia and Lesk (Chothia and Lesk, Mol. Biol. 196:901-917, 1987). There are six HVRs, three in VH (H1, H2, H3) and three in VL (L1, L2, L3). CDRs as defined by Kabat were used herein except in H-CDR1, which is extended to include H1.

There are five types of mammalian immunoglobulin (Ig) heavy-chains, denoted by the Greek letters α (alpha), δ (delta), ε (epsilon), γ (gamma), and μ (mu), which define the class or isotype of an antibody as IgA, IgD, IgE, IgG, or IgM, respectively. IgG antibodies can be further divided into subclasses, for example, IgG1, IgG2, IgG3, and IgG4.

Each heavy-chain type is characterized by a particular constant region with a sequence well known in the art. The constant region is identical in all antibodies of the same isotype but differs in antibodies of different isotypes. Heavy-chains γ, α, and δ have a constant region composed of three tandem immunoglobulin (Ig) domains, and a hinge region for added flexibility. Heavy-chains μ and ε have a constant region composed of four Ig domains.

The hinge region is a flexible amino acid stretch that links the Fc and Fab portions of an antibody. This region contains cysteine residues that can form disulfide bonds, connecting two heavy-chains together.

The variable region of the heavy-chain differs in antibodies produced by different B cells but is the same for all antibodies produced by a single B cell or B cell clone. The variable region of each heavy-chain is approximately 110 amino acids long and is composed of a single Ig domain.

In mammals, light-chains are classified as kappa (κ) or lambda (λ), and are characterized by a particular constant region as known in the art. A light-chain has two successive domains: one variable domain at the amino-terminal end, and one constant domain at the carboxy-terminal end. Each antibody contains two light-chains that are always identical; only one type of light-chain, κ or λ, is present per antibody in mammals.

The Fc region, composed of two heavy-chains that contribute three or four constant domains depending on the class of the antibody, plays a role in modulating immune cell activity. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects, including opsonization, cell lysis, and degranulation of mast cells, basophils and eosinophils.

As used herein, the term “epitope” refers to a specific arrangement of amino acids located on a peptide or protein to which an antibody or antibody fragment binds. Epitopes often consist of a chemically active surface grouping of molecules such as amino acids or sugar side chains and have specific three-dimensional structural characteristics as well as specific charge characteristics. Epitopes can be linear, i.e., involving binding to a single sequence of amino acids, or conformational, i.e., involving binding to two or more sequences of amino acids in various regions of the antigen that may not necessarily be contiguous in the linear sequence.

As used herein, the terms “specifically binds”, “bind specifically”, “specific binding”, and the like as applied to the present antibody compounds refer to the ability of a specific binding agent (such as an antibody) to bind to a target molecular species in preference to binding to other molecular species with which the specific binding agent and target molecular species are admixed. A specific binding agent is said specifically to recognize a target molecular species when it can bind specifically to that target.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

As used herein, the term “sequence identity” means the percentage of identical nucleotide or amino acid residues at corresponding positions in two or more sequences when the sequences are aligned to maximize sequence matching, i.e., considering gaps and insertions. Identity can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, Altschul, S. F. et al., J. Mol. Biol. 215: 403-410 (1990) and Altschul et al. Nucl. Acids Res. 25: 3389-3402 (1997).

One example of an algorithm that is suitable for determining percent sequence identity and sequence similarity is the BLAST algorithm, which is described in (Altschul, S., et al., NCBI NLM NIH Bethesda, Md. 20894; and Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when: the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

As used herein, the terms “humanized”, “humanization”, and the like, refer to grafting of the murine monoclonal antibody CDRs disclosed herein to human FRs and constant regions. Also encompassed by these terms are possible further modifications to the murine CDRs, and human FRs, by the methods disclosed in, for example, Kashmiri et al. (2005) Methods 36(1):25-34 and Hou et al. (2008) J. Biochem. 144(1):115-120, respectively, to improve various antibody properties, as discussed below.

As used herein, the term “humanized antibodies” refers to mAbs and antigen binding fragments thereof, including the antibody compounds disclosed herein, that have binding and functional properties according to the disclosure similar to those disclosed herein, and that have FRs and constant regions that are substantially human or fully human surrounding CDRs derived from a non-human antibody.

As used herein, the term “FR” or “framework sequence” refers to any one of FRs 1 to 4. Humanized antibodies and antigen binding fragments encompassed by the present disclosure include molecules wherein any one or more of FRs 1 to 4 is substantially or fully human, i.e., wherein any of the possible combinations of individual substantially or fully human FRs 1 to 4, is present. For example, this includes molecules in which FR1 and FR2, FR1 and FR3, FR1, FR2, and FR3, etc., are substantially or fully human. Substantially human frameworks are those that have at least 80% sequence identity to a known human germline framework sequence. Preferably, the substantially human frameworks have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity, to a framework sequence disclosed herein, or to a known human germline framework sequence.

Fully human frameworks are those that are identical to a known human germline framework sequence. Human FR germline sequences can be obtained from the international ImMunoGeneTics (IMGT) database and from The Immunoglobulin FactsBook by Marie-Paule Lefranc and Gerard Lefranc, Academic Press, 2001, the contents of which are herein incorporated by reference in their entirety.

The Immunoglobulin Facts Book is a compendium of the human germline immunoglobulin genes that are used to create the human antibody repertoire, and includes entries for 203 genes and 459 alleles, with a total of 837 displayed sequences. The individual entries comprise all the human immunoglobulin constant genes, and germline variable, diversity, and joining genes that have at least one functional or open reading frame allele, and which are localized in the three major loci. For example, germline light-chain FRs can be selected from the group consisting of: IGKV3D-20, IGKV2-30, IGKV2-29, IGKV2-28, IGKV1-27, IGKV3-20, IGKV1-17, IGKV1-16, 1-6, IGKV1-5, IGKV1-12, IGKV1D-16, IGKV2D-28, IGKV2D-29, IGKV3-11, IGKV1-9, IGKV1-39, IGKV1D-39 and IGKV1D-33 and IGKJ1-5 and germline heavy-chain FRs can be selected from the group consisting of: IGHV1-2, IGHV1-18, IGHV1-46, IGHV1-69, IGHV2-5, IGHV2-26, IGHV2-70, IGHV1-3, IGHV1-8, IGHV3-9, IGHV3-11, IGHV3-15, IGHV3-20, IGHV3-66, IGHV3-72, IGHV3-74, IGHV4-31, IGHV3-21, IGHV3-23, IGHV3-30, IGHV3-48, IGHV4-39, IGHV4-59 and IGHV5-51 and IGHJ1-6.

Substantially human FRs are those that have at least 80% sequence identity to a known human germline FR sequence. Preferably, the substantially human frameworks have at least 85%, at least 90%, at least 95%, at least 96%, at least 97%, at least 98%, or at least 99% sequence identity, to a framework sequences disclosed herein, or to a known human germline framework sequence.

CDRs encompassed by the present disclosure include not only those specifically disclosed herein, but also CDR sequences having sequence identities of at least 80%, at least 85%, at least 90%, at least 95%, at least 96%, at least 97%, at least 98%, or at least 99% sequence identity to a CDR sequence disclosed herein. Alternatively, CDRs encompassed by the present disclosure include not only those specifically disclosed herein, but also CDR sequences having 1, 2, 3, 4, or 5 amino acid changes at corresponding positions compared to CDR sequences disclosed herein. Such sequence identical, or amino acid modified, CDRs preferably bind to the antigen recognized by the intact antibody.

Humanized antibodies in addition to those disclosed herein exhibiting similar functional properties according to the present disclosure can be generated using several different methods Almagro et al. Frontiers in Biosciences. Humanization of antibodies. (2008) Jan. 1; 13:1619-33. In one approach, the parent antibody compound CDRs are grafted into a human framework that has a high sequence identity with the parent antibody compound framework. The sequence identity of the new framework will generally be at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identical to the sequence of the corresponding framework in the parent antibody compound. In the case of frameworks having fewer than 100 amino acid residues, one, two, three, four, five, six, seven, eight, nine, or ten amino acid residues can be changed. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. Bioinformatics. 2015 Feb. 1; 31(3):434-435 and U.S. Pat. Nos. 4,816,397, 5,225,539, and 5,693,761; and the method of Winter and co-workers (Jones et al. (1986) Nature 321:522-525; Riechmann et al. (1988) Nature 332:323-327; and Verhoeyen et al. (1988) Science 239:1534-1536.

Humanization began with chimerization, a method developed during the first half of the 1980's (Morrison, S. L., M. J. Johnson, L. A. Herzenberg & V. T. Oi: Chimeric human antibody molecules: mouse antigen-binding domains with human constant region domains. Proc. Natl. Acad. Sci. USA., 81, 6851-5 (1984)), consisting of combining the variable (V) domains of murine antibodies with human constant (C) domains to generate molecules with ˜70% of human content.

Several different methods can be used to generate humanized antibodies, which are described herein. In one approach, the parent antibody compound CDRs are grafted into a human FR that has a high sequence identity with the parent antibody compound framework. The sequence identity of the new FR will generally be at least 80%, at least 85%, at least 90%, at least 95%, at least 96%, at least 97%, at least 98%, or at least 99% identical to the sequence of the corresponding FR in the parent antibody compound. In the case of FRs having fewer than 100 amino acid residues, one, two, three, four, five, or more amino acid residues can be changed. This grafting may result in a reduction in binding affinity compared to that of the parent antibody. If this is the case, the FR can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. Bioinformatics. 2015 Feb. 1; 31(3):434-435 and U.S. Pat. Nos. 4,816,397, 5,225,539, and 5,693,761; and the method of Winter and co-workers (Jones et al. (1986) Nature 321:522-525; Riechmann et al. (1988) Nature 332:323-327; and Verhoeyen et al. (1988) Science 239:1534-1536.

The identification of residues to consider for back-mutation can be carried out as described below. When an amino acid falls under the following category, the framework amino acid of the human germ-line sequence that is being used (the “acceptor FR”) is replaced by a framework amino acid from a framework of the parent antibody compound (the “donor FR”):
(a) the amino acid in the human FR of the acceptor framework is unusual for human frameworks at that position, whereas the corresponding amino acid in the donor immunoglobulin is typical for human frameworks at that position;
(b) the position of the amino acid is immediately adjacent to one of the CDRs; or
(c) any side chain atom of a framework amino acid is within about 5-6 angstroms (center-to-center) of any atom of a CDR amino acid in a three-dimensional immunoglobulin model.

When each of the amino acids in the human FR of the acceptor framework and a corresponding amino acid in the donor framework is generally unusual for human frameworks at that position, such amino acid can be replaced by an amino acid typical for human frameworks at that position. This back-mutation criterion enables one to recover the activity of the parent antibody compound.

Another approach to generating humanized antibodies exhibiting similar functional properties to the antibody compounds disclosed herein involves randomly mutating amino acids within the grafted CDRs without changing the framework and screening the resultant molecules for binding affinity and other functional properties that are as good as, or better than, those of the parent antibody compounds. Single mutations can also be introduced at each amino acid position within each CDR, followed by assessing the effects of such mutations on binding affinity and other functional properties. Single mutations producing improved properties can be combined to assess their effects in combination with one another.

Further, a combination of both of the foregoing approaches is possible. After CDR grafting, one can back-mutate specific FRs in addition to introducing amino acid changes in the CDRs. This methodology is described in Wu et al. (1999) J. Mol. Biol. 294: 151-162.

Applying the teachings of the present disclosure, a person skilled in the art can use common techniques, e.g., site-directed mutagenesis, to substitute amino acids within the presently disclosed CDR and FR sequences and thereby generate further variable region amino acid sequences derived from the present sequences. Up to all naturally occurring amino acids can be introduced at a specific substitution site. The methods disclosed herein can then be used to screen these additional variable region amino acid sequences to identify sequences having the indicated in vivo functions. In this way, further sequences suitable for preparing humanized antibodies and antigen-binding portions thereof in accordance with the present disclosure can be identified. Preferably, amino acid substitution within the frameworks is restricted to one, two, three, four, or five positions within any one or more of the four light-chain and/or heavy-chain FRs disclosed herein. Preferably, amino acid substitution within the CDRs is restricted to one, two, three, four, or five positions within any one or more of the three light-chain and/or heavy-chain CDRs. Combinations of the various changes within these FRs and CDRs described above are also possible.

That the functional properties of the antibody compounds generated by introducing the amino acid modifications discussed above conform to those exhibited by the specific molecules disclosed herein can be confirmed by the methods in Examples disclosed herein.

As described above, to circumvent the problem of eliciting human anti-murine antibody (HAMA) response in patients, murine antibodies have been genetically manipulated to progressively replace their murine content with the amino acid residues present in their human counterparts by grafting their complementarity determining regions (CDRs) onto the variable light (V_{L}) and variable heavy (V_{H}) frameworks of human immunoglobulin molecules, while retaining those murine framework residues deemed essential for the integrity of the antigen-combining site. However, the xenogeneic CDRs of the humanized antibodies may evoke anti-idiotypic (anti-Id) response in patients.

To minimize the anti-Id response, a procedure to humanize xenogeneic antibodies by grafting onto the human frameworks only the CDR residues most crucial in the antibody-ligand interaction, called “SDR grafting”, has been developed, wherein only the crucial specificity determining residues (SDRs) of CDRS are grafted onto the human frameworks. This procedure, described in Kashmiri et al. (2005) Methods 36(1):25-34, involves identification of SDRs through the help of a database of the three-dimensional structures of the antigen-antibody complexes of known structures, or by mutational analysis of the antibody-combining site. An alternative approach to humanization involving retention of more CDR residues is based on grafting of the ‘abbreviated’ CDRs, the stretches of CDR residues that include all the SDRs. Kashmiri et al. also discloses a procedure to assess the reactivity of humanized antibodies to sera from patients who had been administered the murine antibody.

Another strategy for constructing human antibody variants with improved immunogenic properties is disclosed in Hou et al. (2008) J. Biochem. 144(1):115-120. These authors developed a humanized antibody from 4C8, a murine anti-human CD34 monoclonal antibody, by CDR grafting using a molecular model of 4C8 built by computer-assisted homology modelling. Using this molecular model, the authors identified FR residues of potential importance in antigen binding. A humanized version of 4C8 was generated by transferring these key murine FR residues onto a human antibody framework that was selected based on homology to the murine antibody FR, together with the murine CDR residues. The resulting humanized antibody was shown to possess antigen-binding affinity and specificity similar to that of the original murine antibody, suggesting that it might be an alternative to murine anti-CD34 antibodies routinely used clinically.

Embodiments of the present disclosure encompass antibodies created to avoid recognition by the human immune system containing CDRs disclosed herein in any combinatorial form such that contemplated mAbs can contain the set of CDRs from a single murine mAb disclosed herein, or light and heavy-chains containing sets of CDRs comprising individual CDRs derived from two or three of the disclosed murine mAbs. Such mAbs can be created by standard techniques of molecular biology and screened for desired activities using assays described herein. In this way, the disclosure provides a “mix and match” approach to create novel mAbs comprising a mixture of CDRs from the disclosed murine mAbs to achieve new, or improved, therapeutic activities.

Monoclonal antibodies or antigen-binding fragments thereof encompassed by the present disclosure that “compete” with the molecules disclosed herein are those that bind human SIRPα at site(s) that are identical to, or overlapping with, the site(s) at which the present molecules bind. Competing monoclonal antibodies or antigen-binding fragments thereof can be identified, for example, via an antibody competition assay. For example, a sample of purified or partially purified human SIRPα extracellular domain can be bound to a solid support. Then, an antibody compound, or antigen binding fragment thereof, of the present disclosure and a monoclonal antibody or antigen-binding fragment thereof suspected of being able to compete with such disclosure antibody compound are added. One of the two molecules is labeled. If the labeled compound and the unlabeled compound bind to separate and discrete sites on SIRPα, the labeled compound will bind to the same level whether or not the suspected competing compound is present. However, if the sites of interaction are identical or overlapping, the unlabeled compound will compete, and the amount of labeled compound bound to the antigen will be lowered. If the unlabeled compound is present in excess, very little, if any, labeled compound will bind. For purposes of the present disclosure, competing monoclonal antibodies or antigen-binding fragments thereof are those that decrease the binding of the present antibody compounds to SIRPα by about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, or about 99%. Details of procedures for carrying out such competition assays are well known in the art and can be found, for example, in Harlow and Lane (1988) Antibodies, A Laboratory Manual, Cold Spring Harbor Laboratory Press, Cold Spring Harbor, N.Y. Such assays can be made quantitative by using purified antibodies. A standard curve is established by titrating one antibody against itself, i.e., the same antibody is used for both the label and the competitor. The capacity of an unlabeled competing monoclonal antibody or antigen-binding fragment thereof to inhibit the binding of the labeled molecule to the plate is titrated. The results are plotted, and the concentrations necessary to achieve the desired degree of binding inhibition are compared.

Whether mAbs or antigen-binding fragments thereof that compete with antibody compounds of the present disclosure in such competition assays possess the same or similar functional properties of the present antibody compounds can be determined via these methods in conjunction with the methods described in Examples 2-7, below. In various embodiments, competing antibodies for use in the therapeutic methods encompassed herein possess biological activities as described herein in the range of from about 50% to about 100% or about 125%, or more, compared to that of the antibody compounds disclosed herein. In some embodiments, competing antibodies possess about 50%, about 60%, about 70%, about 80%, about 85%, about 90%, about 91%, about 92%, about 93%, about 94%, about 95%, about 96%, about 97%, about 98%, about 99%, or identical biological activity compared to that of the antibody compounds disclosed herein as determined by the methods disclosed in the Examples presented below.

The mAbs or antigen-binding fragments thereof or competing antibodies useful in the compositions and methods can be any of the isotypes described herein. Furthermore, any of these isotypes can comprise further amino acid modifications as follows.

The monoclonal antibody or antigen-binding fragment thereof, or competing antibody described herein can be of the human IgG1 isotype.

The human IgG1 constant region of the monoclonal antibody, antigen-binding fragment thereof, or competing antibody described herein can be modified to alter antibody half-life. Antibody half-life is regulated in large part by Fc-dependent interactions with the neonatal Fc receptor (Roopenian and Alikesh, 2007). The human IgG1 constant region of the monoclonal antibody, antigen-binding fragment thereof, or competing antibody can be modified to increase half-life include, but are not limited to amino acid modifications N434A, T307A/E380A/N434A (Petkova et al., 2006, Yeung et al., 2009); M252Y/S254T/T256E (Dall'Acqua et al., 2006); T250Q/M428L (Hinton et al., 2006); and M428L/N434S (Zalevsky et al., 2010).

As opposed to increasing half-life, there are some circumstances where decreased half-life would be desired, such as to reduce the possibility of adverse events associated with high Antibody-Dependent Cellular Cytotoxicity (ADCC) and Complement-Dependent Cytotoxicity (CDC) antibodies (Presta 2008). The human IgG1 constant region of the monoclonal antibody, antigen-binding fragment thereof, or competing antibody described herein can be modified to decrease half-life and/or decrease endogenous IgG include, but are not limited to, amino acid modifications I253A (Petkova et al., 2006); P257I/N434H, D376V/N434H (Datta-Mannan et al., 2007); and M252Y/S254T/T256E/H433K/N434F (Vaccaro et al., 2005).

The human IgG1 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), C1q binding, and altered binding to Fc receptors.

The human IgG1 constant region of the monoclonal antibody, antigen-binding fragment thereof, or competing antibody described herein can be modified to increase antibody effector function include, but are not limited to amino acid modifications S298A/E333A/K334 (Shields et al., 2001); S239D/I332E and S239D/A330L/I332E (Lazar et al., 2006); F234L/R292P/Y300L, F234L/R292P/Y300L/P393L, and F243L/R292P/Y300L/V305I/P396L (Stevenhagen et al., 2007); G236A, G236A/S239D/I332E, and G236A/S239D/A330L/I332E (Richards et al., 2008); K326A/E333A, K326A/E333S and K326W/E333S (Idusogie et al., 2001); S267E and S267E/L328F (Smith et al., 2012); H268F/S324T, S267E/H268F, S267E/S234T, and S267E/H268F/S324T (Moore et al., 2010); S298G/T299A (Sazinsky et al., 2008); E382V/M428I (Jung et al., 2010).

The human IgG1 constant region of the monoclonal antibody, antigen-binding fragment thereof, or competing antibody described herein can be modified to decrease antibody effector function include, but are not limited to amino acid modifications N297A and N297Q (Bolt et al., 1993, Walker et al., 1989); L234A/L235A (Xu et al., 2000); K214T/E233P/L234V/L235A/G236-deleted/A327G/P331A/D356E/L358M (Ghevaert et al., 2008); C226S/C229S/E233P/L234V/L235A (McEarchern et al., 2007); S267E/L328F (Chu et al., 2008).

The human IgG1 constant region of the monoclonal antibody, antigen-binding fragment thereof, or competing antibody described herein can be modified to decrease antibody effector function include, but are not limited to amino acid modifications V234A/G237A (Cole et al., 1999); E233D, G237D, P238D, H268Q, H268D, P271G, V309L, A330S, A330R, P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The monoclonal antibody or antigen-binding fragment thereof, or competing antibody described herein can be of the human IgG2 isotype.

The human IgG2 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), and C1q binding, and altered binding to Fc receptors.

The human IgG2 constant region of the monoclonal antibody, antigen-binding fragment thereof, or a competing antibody described herein, can be modified to increase antibody effector function include, but are not limited to, the amino acid modification K326A/E333S (Idusogie et al., 2001).

The human IgG2 constant region of the monoclonal antibody, antigen-binding fragment thereof, or competing antibody described herein can be modified to decrease antibody effector function include, but are not limited to amino acid modifications V234A/G237A (Cole et al., 1999); E233D, G237D, P238D, H268Q, H268D, P271G, V309L, A330S, A330R, P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The Fc region of a human IgG2 of the monoclonal antibody, antigen-binding fragment thereof, or competing antibody described herein can be modified to alter isoform and/or agonistic activity, include, but are not limited to amino acid modifications C127S (C_{H1} domain), C232S, C233S, C232S/C233S, C236S, and C239S (White et al., 2015, Lightle et al., 2010).

The Fc region of a human IgG2 of the monoclonal antibody, antigen-binding fragment thereof, or competing antibody described herein can be modified to exhibit diminished FcγR binding capacity but have conserved FcRn binding. These IgG Fc mutants enable therapeutic targeting of soluble or cell surface antigens while minimizing Fc-associated engagement of immune effector function and complement mediated cytotoxicity. In one embodiment, the IgG2 Fc mutant comprises V234A, G237A, P238S according to the EU numbering system. In another embodiment, the IgG2 Fc mutant comprises V234A, G237A, H268Q, or H268A, V309L, A330S, P331S, according to the EU numbering system. In a particular aspect, the IgG2 Fc mutant comprises V234A, G237A, P238S, H268A, V309L, A330S, P331S, and, optionally, P233S according to the EU numbering system.

The monoclonal antibody or antigen-binding fragment thereof, or competing antibody described herein can be of the human IgG3 isotype.

The human IgG3 constant region of the monoclonal antibody, or antigen binding fragment thereof, wherein said human IgG3 constant region of the monoclonal antibody, or antigen-binding fragment thereof can be modified at one or more amino acid(s) to increase antibody half-life, Antibody-Dependent Cellular Cytotoxicity (ADCC), Complement-Dependent Cytotoxicity (CDC), or apoptosis activity.

The human IgG3 constant region of the monoclonal antibody, or antigen-binding fragment thereof, wherein said human IgG3 constant region of the monoclonal antibody, or antigen-binding fragment thereof can be modified at amino acid R435H to increase antibody half-life.

The monoclonal antibody or antigen-binding fragment thereof, or competing antibody described herein can be of the human IgG4 isotype.

The human IgG4 constant region of the monoclonal antibody, antigen-binding fragment thereof, or competing antibody described herein can be modified to decrease antibody effector functions. These antibody effector functions include, but are not limited to, Antibody-Dependent Cellular Cytotoxicity (ADCC) and Antibody-Dependent Cellular Phagocytosis (ADCP).

The human IgG4 constant region of the monoclonal antibody, antigen-binding fragment thereof, or competing antibody described herein can be modified to prevent Fab arm exchange and/or decrease antibody effector function include, but are not limited to, amino acid modifications F234A/L235A (Alegre et al., 1994); S228P, L235E and S228P/L235E (Reddy et al., 2000).

As used herein, the term “tumor” refers to all neoplastic cell growth and proliferation, whether malignant or benign, and all pre-cancerous and cancerous cells and tissues.

The terms “cancer”, “cancerous”, and “tumor” are not mutually exclusive as used herein.

The terms “cancer” and “cancerous” refer to or describe the physiological condition in mammals that is typically characterized by aberrant cell growth/proliferation. Examples of cancers include, but are not limited to, carcinoma, lymphoma (i.e., Hodgkin's and non-Hodgkin's lymphoma), multiple myeloma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma, leukemia and other lymphoproliferative disorders, and various types of head and neck cancer.

The term “susceptible cancer” as used herein refers to a cancer, cells of which express CD47, IRPα, or CD47 and SIRPα and are responsive to treatment with an antibody or antigen binding fragment thereof, or competing antibody or antigen binding fragment thereof, from the present disclosure that prevent interaction between CD47 and SIRPα.

The term “autoimmune disease” as used herein refers to when the body's immune system turns against itself and mistakenly attacks healthy cells.

The term “inflammatory disease” as used herein refers to a disease characterized by inflammation which is a fundamental pathologic process consisting of a dynamic complex of histologically apparent cytologic changes, cellular infiltration, and mediator release that occurs in the affected blood vessels and adjacent tissues in response to an injury or abnormal stimulation caused by a physical, chemical, or biologic agent, including the local reactions and resulting morphologic changes; the destruction or removal of the injurious material; and the responses that lead to repair and healing.

The term “autoinflammatory disease” as used herein refers to a disease that results when the innate immune system causes inflammation for unknown reasons.

As used herein, term “treating” or “treat” or “treatment” means slowing, interrupting, arresting, controlling, stopping, reducing, or reversing the progression or severity of a sign, symptom, disorder, condition, or disease, but does not necessarily involve a total elimination of all disease-related signs, symptoms, conditions, or disorders. The term “treating” and the like refer to a therapeutic intervention that ameliorates a sign or symptom of a disease or pathological condition after it has begun to develop.

As used herein, term “effective amount” refers to the amount or dose of an antibody compound of the present disclosure which, upon single or multiple dose administration to a patient or organ, provides the desired treatment or prevention.

The precise effective amount for any particular subject will depend upon their size and health, the nature and extent of their condition, and the therapeutics or combination of therapeutics selected for administration. The effective amount for a given patient is determined by routine experimentation and is within the judgment of a clinician. Therapeutically effective amounts of the present antibody compounds can also comprise an amount in the range of from about 0.1 mg/kg to about 150 mg/kg, from about 0.1 mg/kg to about 100 mg/kg, from about 0.1 mg/kg to about 50 mg/kg, or from about 0.05 mg/kg to about 10 mg/kg per single dose administered to a harvested organ or to a patient. Known antibody-based pharmaceuticals provide guidance in this respect. For example, Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m²; for example.

A therapeutically effective amount for any individual patient can be determined by the health care provider by monitoring the effect of the antibody compounds on tumor regression, circulating tumor cells, tumor stem cells or anti-tumor responses. Analysis of the data obtained by these methods permits modification of the treatment regimen during therapy so that optimal amounts of antibody compounds of the present disclosure, whether employed alone or in combination with one another, or in combination with another therapeutic agent, or both, are administered, and so that the duration of treatment can be determined as well. In this way, the dosing/treatment regimen can be modified over the course of therapy so that the lowest amounts of antibody compounds used alone or in combination that exhibit satisfactory efficacy are administered, and so that administration of such compounds is continued only so long as is necessary to successfully treat the patient. Known antibody-based pharmaceuticals provide guidance relating to frequency of administration e.g., whether a pharmaceutical should be delivered daily, weekly, monthly, etc. Frequency and dosage may also depend on the severity of symptoms.

In some embodiments, antibody compounds of the present disclosure can be used as medicaments in human and veterinary medicine, administered by a variety of routes including, but not limited to, oral, intravenous, intramuscular, intra-arterial, intramedullary, intraperitoneal, intrathecal, intraventricular, transdermal, transcutaneous, topical, subcutaneous, intratumoral, intranasal, enteral, sublingual, intravaginal, intravesicular or rectal routes. The compositions can also be administered directly into a lesion such as a tumor. Dosage treatment may be a single dose schedule or a multiple dose schedule. Hypo sprays may also be used to administer the pharmaceutical compositions. Typically, the therapeutic compositions can be prepared as injectables, either as liquid solutions or suspensions. Solid forms suitable for solution in, or suspension in, liquid vehicles prior to injection can also be prepared. Veterinary applications include the treatment of companion/pet animals, such as cats and dogs; working animals, such as guide or service dogs, and horses; sport animals, such as horses and dogs; zoo animals, such as primates, cats such as lions and tigers, bears, etc.; and other valuable animals kept in captivity.

Such pharmaceutical compositions can be prepared by methods well known in the art. See, e.g., Remington: The Science and Practice of Pharmacy, 21^{st} Edition (2005), Lippincott Williams & Wilkins, Philadelphia, Pa., and comprise one or more antibody compounds disclosed herein, and a pharmaceutically acceptable, for example, physiologically acceptable, carrier, diluent, or excipient.

### Cancer Indications

Presently disclosed are anti-SIRPα mAbs and antigen binding fragments thereof effective as cancer therapeutics which can be administered to patients, preferably parenterally, with susceptible hematologic cancers and solid tumors including, but not limited to, leukemias, including systemic mastocytosis, acute lymphocytic (lymphoblastic) leukemia (ALL), T-cell—ALL, acute myeloid leukemia (AML), myelogenous leukemia, chronic lymphocytic leukemia (CLL), chronic myeloid leukemia (CML), myeloproliferative disorder/neoplasm, monocytic cell leukemia, and plasma cell leukemia; multiple myeloma (MM); Waldenstrom's Macroglobulinemia; lymphomas, including histiocytic lymphoma and T-cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL; solid tumors, including ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma (liver cancer, hepatoma), gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, myelodysplastic syndrome, and sarcomas including, but not limited to, osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma; and melanoma.

### Treatment of Cancer

As is well known to those of ordinary skill in the art, combination therapies are often employed in cancer treatment as single-agent therapies or procedures may not be sufficient to treat or cure the disease or condition. Conventional cancer treatments often involve surgery, radiation treatment, a combination of cytotoxic drugs to achieve additive or synergistic effects, or combinations of any or all of these approaches. Especially useful chemotherapeutic and biologic therapy combinations employ drugs that work via different mechanisms of action, increasing cancer cell control or killing, increasing the ability of the immune system to control cancer cell growth, reducing the likelihood of drug resistance during therapy, and minimizing possible overlapping toxicities by permitting the use of reduced doses of individual drugs.

Classes of conventional anti-tumor and anti-neoplastic agents useful in the combination therapies encompassed by the present methods are disclosed in Goodman & Gilman's The Pharmacological Basis of Therapeutics, Twelfth Edition (2010) L. L. Brunton, B. A. Chabner, and B. C. Knollmann Eds., Section VIII, “Chemotherapy of Neoplastic Diseases”, Chapters 60-63, pp. 1665-1770, McGraw-Hill, NY, include but are not limited to anthracyclines, platinums, taxols, topisomerase inhibitors, anti-metabolites, anti-tumor antibiotics, mitotic inhibitors, and alkylating agents.

In addition to the foregoing, the methods of the present disclosure are related to treatment of cancer indications and further comprises treating the patient via surgery, radiation, and/or administering to a patient in need thereof an effective amount of a chemical small molecule or biologic drug including, but not limited to, a peptide, polypeptide, protein, nucleic acid therapeutic, conventionally used or currently being developed, to treat tumorous conditions. This includes antibodies and antigen-binding fragments, other than those disclosed herein, cytokines, antisense oligonucleotides, siRNAs, and miRNAs.

The therapeutic methods disclosed and claimed herein include the use of the antibodies disclosed herein alone, and/or in combinations with one another, and/or with antigen-binding fragments thereof of the present disclosure that bind to SIRPα, and/or with competing antibodies exhibiting appropriate biological/therapeutic activity, as well, for example, all possible combinations of these antibody compounds to achieve the greatest treatment efficacy.

In addition, the present therapeutic methods also encompass the use of these antibodies, antigen-binding fragments thereof, competing antibodies, and combinations thereof in further in combination with: (1) one or more anti-tumor therapeutic treatments selected from surgery, radiation, anti-tumor, and anti-neoplastic agents or combinations of any of these, or (2) one or more of anti-tumor biological agents or (3) equivalents of any of the foregoing of (1) or (2) as would be apparent to one of ordinary skill in the art, in appropriate combination(s) to achieve the desired therapeutic treatment effect for the particular indication.

Antibodies and small molecule drugs that increase the immune response to cancer by modulating co-stimulatory or inhibitory interactions that influence the T-cell response to tumor antigens, including inhibitors of immune checkpoints and modulators of co-stimulatory molecules, are also of particular interest in the context of the combination therapeutic methods encompassed herein and include, but are not limited to, other anti-SIRPα antibodies. Administration of therapeutic agents that bind to the SIRPα protein, for example, antibodies or small molecules that bind to SIRPα and prevent interaction between CD47 and SIRPα, are administered to a patient, causing the clearance of cancer cells via phagocytosis. The therapeutic agent that binds to the SIRPα protein is combined with a therapeutic agent such as an antibody, a chemical small molecule or biologic drug which is directed against one or more additional cellular targets selected from CD47 (Cluster of Differentiation 47), CD70 (Cluster of Differentiation 70), CD200 (OX-2 membrane glycoprotein, Cluster of Differentiation 200), CD154 (Cluster of Differentiation 154, CD40L, CD40 ligand, Cluster of Differentiation 40 ligand), CD223 (Lymphocyte-activation gene 3, LAG3, Cluster of Differentiation 223), KIR (Killer-cell immunoglobulin-like receptors), GITR (TNFRSF18, glucocorticoid-induced TNFR-related protein, activation-inducible TNFR family receptor, AITR, Tumor necrosis factor receptor superfamily member 18), CD20 (Cluster of Differentiation 20), CD28 (Cluster of Differentiation 28), CD40 (Cluster of Differentiation 40, Bp50, CDW40, TNFRSF5, Tumor necrosis factor receptor superfamily member 5, p50), CD86 (B7-2, Cluster of Differentiation 86), CD160 (Cluster of Differentiation 160, BY55, NK1, NK28), CD258 (LIGHT, Cluster of Differentiation 258, Tumor necrosis factor ligand superfamily member 14, TNFSF14, herpesvirus entry mediator ligand (HVEM-L), CD270 (HVEM, Tumor necrosis factor receptor superfamily member 14, herpesvirus entry mediator, Cluster of Differentiation 270, LIGHTR, HVEA), CD275 (ICOSL, ICOS ligand, Inducible T-cell co-stimulator ligand, Cluster of Differentiation 275), CD276 (B7-H3, B7 homolog 3, Cluster of Differentiation 276), OX40L (OX40 Ligand), B7-H4 (B7 homolog 4, VTCN1, V-set domain-containing T-cell activation inhibitor 1), GITRL (Glucocorticoid-induced tumor necrosis factor receptor-ligand, glucocorticoid-induced TNFR-ligand), 4-1BBL (4-1BB ligand), CD3 (Cluster of Differentiation 3, T3D), CD25 (IL2Rα, Cluster of Differentiation 25, Interleukin-2 Receptor a chain, IL-2 Receptor a chain), CD48 (Cluster of Differentiation 48, B-lymphocyte activation marker, BLAST-1, signaling lymphocytic activation molecule 2, SLAMF2), CD66a (Ceacam-1, Carcinoembryonic antigen-related cell adhesion molecule 1, biliary glycoprotein, BGP, BGP1, BGPI, Cluster of Differentiation 66a), CD80 (B7-1, Cluster of Differentiation 80), CD94 (Cluster of Differentiation 94), NKG2A (Natural killer group 2A, killer cell lectin-like receptor subfamily D member 1, KLRD1), CD96 (Cluster of Differentiation 96, TActILE, T-cell activation increased late expression), CD112 (PVRL2, nectin, Poliovirus receptor-related 2, herpesvirus entry mediator B, HVEB, nectin-2, Cluster of Differentiation 112), CD115 (CSF1R, Colony stimulating factor 1 receptor, macrophage colony-stimulating factor receptor, M-CSFR, Cluster of Differentiation 115), CD205 (DEC-205, LY75, Lymphocyte antigen 75, Cluster of Differentiation 205), CD226 (DNAM1, Cluster of Differentiation 226, DNAX Accessory Molecule-1, PTA1, platelet and T-cell activation antigen 1), CD244 (Cluster of Differentiation 244, Natural killer cell receptor 2B4), CD262 (DR5, TrailR2, TRAIL-R2, Tumor necrosis factor receptor superfamily member 10b, TNFRSF10B, Cluster of Differentiation 262, KILLER, TRICK2, TRICKB, ZTNFR9, TRICK2A, TRICK2B), CD284 (Toll-like Receptor-4, TLR4, Cluster of Differentiation 284), CD288 (Toll-like Receptor-8, TLR8, Cluster of Differentiation 288), Leukemia Inhibitor Factor (LIF), TNFSF15 (Tumor necrosis factor superfamily member 15, Vascular endothelial growth inhibitor, VEGI, TL1A), TDO2 (Tryptophan 2,3-dioxygenase, TPH2, TRPO), IGF-1R (Type 1 Insulin-like Growth Factor), GD2 (Disialoganglioside 2), TMIGD2 (Transmembrane and immunoglobulin domain-containing protein 2), RGMB (RGM domain family, member B), VISTA (V-domain immunoglobulin-containing suppressor of T-cell activation, B7-H5, B7 homolog 5), BTNL2 (Butyrophilin-like protein 2), Btn (Butyrophilin family), TIGIT (T-cell Immunoreceptor with Ig and ITIM domains, Vstm3, WUCAM), Siglecs (Sialic acid binding Ig-like lectins), i.e., SIGLEC-15, Neurophilin, VEGFR (Vascular endothelial growth factor receptor), ILT family (LIRs, immunoglobulin-like transcript family, leukocyte immunoglobulin-like receptors), NKG families (Natural killer group families, C-type lectin transmembrane receptors), MICA (MHC class I polypeptide-related sequence A), TGFβ (Transforming growth factor β), STING pathway (Stimulator of interferon gene pathway), Arginase (Arginine amidinase, canavanase, L-arginase, arginine transamidinase), EGFRvIII (Epidermal growth factor receptor variant III), and HHLA2 (B7-H7, B7y, HERV-H LTR-associating protein 2, B7 homolog 7), inhibitors of PD-1 (Programmed cell death protein 1, PD-1, CD279, Cluster of Differentiation 279), PD-L1 (B7-H1, B7 homolog 1, Programmed death-ligand 1, CD274, cluster of Differentiation 274), PD-L2 (B7-DC, Programmed cell death 1 ligand 2, PDCDILG2, CD273, Cluster of Differentiation 273), CTLA-4 (Cytotoxic T-lymphocyte-associated protein 4, CD152, Cluster of Differentiation 152), BTLA (B- and T-lymphocyte attenuator, CD272, Cluster of Differentiation 272), Indoleamine 2,3-dioxygenase (IDO, IDO1), TIM3 (HAVCR2, Hepatitis A virus cellular receptor 2, T-cell immunoglobulin mucin-3, KIM-3, Kidney injury molecule 3, TIMD-3, T-cell immunoglobulin mucin-domain 3), A2A adenosine receptor (ADO receptor), CD39 (ectonucleoside triphosphate diphosphohydrolase-1, Cluster of Differentiation 39, ENTPD1), and CD73 (Ecto-5′-nucleotidase, 5′-nucleotidase, 5′-NT, Cluster of Differentiation 73), CD27 (Cluster of Differentiation 27), ICOS (CD278, Cluster of Differentiation 278, Inducible T-cell Co-stimulator), CD137 (4-1BB, Cluster of Differentiation 137, tumor necrosis factor receptor superfamily member 9, TNFRSF9), OX40 (CD134, Cluster of Differentiation 134), TNFSF25 (Tumor necrosis factor receptor superfamily member 25), IL-10 (Interleukin-10, human cytokine synthesis inhibitory factor, CSIF), and Galectins.

ERBITUX® (cetuximab, Bristol-Meyers Squibb) is an example of an approved recombinant, human/mouse chimeric monoclonal antibody that binds specifically to the extracellular domain of the human epidermal growth factor receptor (EGFR).

RITUXAN® (rituximab, Biogen IDEC/Genentech) is an example of an approved anti-CD20 antibody.

YERVOY® (ipilimumab; Bristol-Meyers Squibb) is an example of an approved anti-CTLA-4 antibody.

KEYTRUDA® (pembrolizumab; Merck) and OPDIVO® (nivolumab; Bristol-Meyers Squibb Company) are examples of approved anti-PD-1 antibodies.

TECENTRIQ™ (atezolizumab; Roche) is an example of an approved anti-PD-L1 antibody.

BAVENCIO™ (avelumab; Merck KGaA and Pfizer and Eli Lilly and Company) is an example of an approved anti-PD-L1 antibody.

IMFINZI™ (Durvalumab; Medimmune/AstraZeneca) is an example of an approved anti-PD-L1 antibody.

The Examples illustrate various embodiments of the present disclosure, but they should not be considered as limiting the disclosure to only these particularly disclosed embodiments.

### EXAMPLES

### Example 1

### Amino Acid Sequences

| | | |
|---|---|---|
| LCDR1 | LCDR2 | LCDR3 |

Light Chain CDRs

| | | | | | |
|---|---|---|---|---|---|
| SEQ ID NO: 1 | RASSGVNYMY | SEQ ID NO: 2 | YTSILAP | SEQ ID NO: 3 | QQFTSSPYT |
| SEQ ID NO: 4 | RASQSIGTSIH | SEQ ID NO: 5 | YGSESIS | SEQ ID NO: 6 | QQSNTWPLT |
| SEQ ID NO: 7 | SASSIIGSDFLH | SEQ ID NO: 8 | RTSILAS | SEQ ID NO: 9 | QQGSGLPLT |
| SEQ ID NO: 10 | KASQDINSHLS | SEQ ID NO: 11 | RANRLAD | SEQ ID NO: 12 | LQYDEFPYT |
| SEQ ID NO: 13 | SASSSVSYMY | SEQ ID NO: 14 | LTSNLAS | SEQ ID NO: 15 | QQWSGNPFT |
| SEQ ID NO: 16 | RASENIYSYLT | SEQ ID NO: 17 | NAKTLAE | SEQ ID NO: 18 | QHHYGSPRT |
| SEQ ID NO: 19 | SASSSISSNFLH | SEQ ID NO: 20 | RTSILAS | SEQ ID NO: 21 | QQGSGLPLT |
| SEQ ID NO: 22 | SSVSY | SEQ ID NO: 23 | DTS | SEQ ID NO: 24 | QQWSSFPWT |
| SEQ ID NO: 25 | EDIYDR | SEQ ID NO: 26 | GTA | SEQ ID NO: 27 | QQYWTTPWT |
| SEQ ID NO: 28 | SSVNY | SEQ ID NO: 29 | YTS | SEQ ID NO: 30 | QQFTSSPFT |
| | | SEQ ID NO: 31 | RANRLAT | SEQ ID NO: 32 | QQYDEFPYT |

Heavy Chain CDRs

| | | | | | |
|---|---|---|---|---|---|
| SEQ ID NO: 33 | KYWIE | SEQ ID NO: 34 | EILPGSVITNYNEKFKG | SEQ ID NO: 35 | WGLYDSDDGVDY |
| SEQ ID NO: 36 | GCTMS | SEQ ID NO: 37 | YISNGGDITYYPDTVKG | SEQ ID NO: 38 | LDGYYYAMDF |
| SEQ ID NO: 39 | SYVMH | SEQ ID NO: 40 | YINPYNDGPKYNEKFKG | SEQ ID NO: 41 | WDYFNSASGFAF |
| SEQ ID NO: 42 | DYFLN | SEQ ID NO: 43 | RINPYNGDSFINQNFRD | SEQ ID NO: 44 | GGYDGYFIAYFDY |
| SEQ ID NO: 45 | SYTMH | SEQ ID NO: 46 | YINPTIGYTEYNQKFKD | SEQ ID NO: 47 | LVITSVLGRAMDY |
| SEQ ID NO: 48 | DYGVN | SEQ ID NO: 49 | WVNTNTRESTYVEDFKG | SEQ ID NO: 50 | GAYDAYYYYYGMDY |
| SEQ ID NO: 51 | TYVMH | SEQ ID NO: 52 | YINPNNDGPNYNEKFKG | SEQ ID NO: 53 | WDSYNSAAGFAY |
| SEQ ID NO: 54 | GFTLSTYT | SEQ ID NO: 55 | ITSGDTYT | SEQ ID NO: 56 | TRDRPLFH |
| SEQ ID NO: 57 | GYTFTDYE | SEQ ID NO: 58 | IHPGSGGT | SEQ ID NO: 59 | TRAVSGYYAMDY |
| SEQ ID NO: 60 | GYTFSNYL | SEQ ID NO: 61 | IYPGDNNT | SEQ ID NO: 62 | AGGTDYDGFAN |
| | | | | SEQ ID NO: 63 | ARAVSGYYAMDY |

Murine Light Chain (V_{L}) Variable Domain Sequences and Human Light Chain (V_{L}) Variable Domain Sequences

| | |
|---|---|
| SEQ ID NO: 64 | ENVLTQSPAIMSASLGEKVTMSCRASSGVNYMYWYQQKSDASPKLLIYYTSILAPGVPARFSGSGSG |
| | NSYSLTISSMEGEDAATYYCQQFTSSPYTFGGGTKLEIK |
| SEQ ID NO: 65 | DILLTQSPAILSVSPGERVSFSCRASQSIGTSIHWYQQRTNGSPRLLIKYGSESISGIPSRFSGSGSGTDFT |
| | LSINSVESEDIADYYCQQSNTWPLTFGDGTKLELK |
| SEQ ID NO: 66 | EIVLTQSPTTMAASPGEKITIICSASSIIGSDFLHWYQQRPGFSPKFLIYRTSILASGVPTRFTGSGSGTSY |
| | SLTIGTMEAEDVATYYCQQGSGLPLTFGSGTKLEMK |
| SEQ ID NO: 67 | DIKLTQSQSSMYSSLGQRVTITCKASQDINSHLSWFQEKPGKSPKTLIYRANRLADGVPSRFSGSGSGQ |
| | DYFLTISSLEYEDVGIYYCLQYDEFPYTFGGGTKLEIK |
| SEQ ID NO: 68 | QIVLTQSPALMSASPGEKVTMTCSASSSVSYMYWFQQKPRSSPKPWIYLTSNLASGVPARFSGSGSGT |
| | SYSLTISSMEAEDAATYYCQQWSGNPFTFGSGTKLEIK |
| SEQ ID NO: 69 | DIQMTQSPASLSASVGETVTITCRASENIYSYLTWYKQKQGKSPQLLVYNAKTLAEGVPSRFSGSGSG |
| | TQFSLKINSLQPEDFGSYYCQHHYGSPRTFGGGTKLEIK |
| SEQ ID NO: 70 | EIVLTQSPTTMAASPGEKITIICSASSSISSNFLHWYQQKPGFSPRFLIYRTSILASGVPTRFSGSGSGTSY |
| | SLTIDTMEAEDVATYYCQQGSGLPLTFGSGTKLEIK |
| SEQ ID NO: 71 | QIVLTQSPAIMSASPGEKVTMTCSASSSVSYMYWYQQKPGSSPRLLIYDTSNLASGVPVRFSGSGSGTS |
| | YSLTISRMEAEDAATYYCQQWSSFPWTFGGGTKLEIK |
| SEQ ID NO: 72 | DIQMTQSSSSFSGSLGDRLTINCKASEDIYDRVAWYQQKPGNAPRLLISGTASLETGVLSRFSGSGSGK |
| | DYTLSINGLQAEDVATYYCQQYWTTPWTFGGGTKLEIK |
| SEQ ID NO: 73 | ENVLTQSPAIMSASLGEKVTMSCRASSSVNYMYWYQQKSDASPKLWIYYTSKLAPGVPARFSGSGSG |
| | NSYSLTISSMEGEDAATYYCQQFTSSPFTFGSGTKLEIK |
| SEQ ID NO: 74 | DIQMTQSPSSLSASVGDRVTITCKASQDINSHLSWYQQKPGKAPKLLIYRANRLATGVPSRFSGSGSGT |
| | DFTFTISSLQPEDIATYYCLQYDEFPYTFGGGTKLEIK |
| SEQ ID NO: 75 | DIQMTQSPSSLSASVGDRVTITCKASQDINSHLSWYQQKPGKAPKLLIYRANRLATGVPSRFSGSGSGT |
| | DFTFTISSLEYEDIATYYCLQYDEFPYTFGGGTKLEIK |
| SEQ ID NO: 76 | DIQMTQSPSSLSASVGDRVTITCKASQDINSHLSWYQQKPGKAPKLLIYRANRLATGVPSRFSGSGSGT |
| | DFTFTISSLQPEDIATYYCQQYDEFPYTFGGGTKLEIK |
| SEQ ID NO: 77 | DIKMTQSPSSMYASLGQRVTITCKASQDINSHLSWFQEKPGKSPKTLIYRANRLADGVPSRFSGSGSG |
| | QDYFLTISSLEYEDVGIYYCLQYDEFPYTFGGGTKLEIK |
| SEQ ID NO: 78 | DIQMTQSPSSLSASVGDRVTITCKASEDIYDRVAWYQQKPGKAPKLLIYGTASLETGVPSRFSGSGSGT |
| | DFTFTISSLQPEDIATYYCQQYWTTPWTFGGGTKVEIK |
| SEQ ID NO: 79 | DIQMTQSPSSLSASVGDRVTITCKASEDIYDRVAWYQQKPGKAPKLLIYGTASLETGVPSRFSGSGSGT |
| | DFTLTISSLQPEDFATYYCQQYWTTPWTFGGGTKVEIK |
| SEQ ID NO: 80 | DIQMTQSPSSLSASVGDRVTITCKASEDIYDRVAWYQQKPGKAPKLLIYGTASLETGVLSRFSGSGSG |
| | TDFTLTISSLQAEDFATYYCQQYWTTPWTFGGGTKVEIK |

Murine Heavy Chain (V_{H}) Variable Domain Sequences and Human Heavy Chain (V_{H}) Variable Domain Sequences

| | |
|---|---|
| SEQ ID NO: 81 | QVQLQQSGAELMKPGASVKISCKATGYSFTKYWIEWVKQRPGHGLEWIGEILPGSVITNYNEKFKGK |
| | ATFTADTSSNTVYMQLSSLTSEDSAVYYCTKWGLYDSDDGVDYWGQGTTLTVSS |
| SEQ ID NO: 82 | EVKLVESGGGLVQPGGSLKLSCAASGFSFSGCTMSWIRQTPERRLEWVAYISNGGDITYYPDTVKGRF |
| | TISRDNAKNSLYLQMSSLKSEDTAMYYCARLDGYYYAMDFWGQGTSVTVSS |
| SEQ ID NO: 83 | EVQLQQSGPEVVKPGASVKMSCKASGYTFTSYVMHWVKQKPGQGLEWIGYINPYNDGPKYNEKFKG |
| | KATLTSDKSSSTAYMELSSLTSEDSAVYFCARWDYFNSASGFAFWGQGTLVTVSA |
| SEQ ID NO: 84 | EVQLQQSGPDLVKPGASVKISCKASGYSFTDYFLNWVKQSHGKSLEWIGRINPYNGDSFINQNFRDKA |
| | TLTVDKSSTTAHMDLLSLTSEDSAIYYCGRGGYDGYFIAYFDYWGQGSLVTVSA |
| SEQ ID NO: 85 | QVQLQQSAAELARPGASVKMSCKASGYTFTSYTMHWVKQRPGQGLEWIGYINPTIGYTEYNQKFKD |
| | KTTLTADKSSSTAYMQLSSLTSEDSAVYYCVRLVITSVLGRAMDYWGQGTSVTVSS |
| SEQ ID NO: 86 | QIQLVQSGPELKKPGETVKISCKASGYTFTDYGVNWVKQGPGKDLQWMGWVNTNTRESTYVEDFKG |
| | RFAFSLETSASTAYLQINNLKNEDSSTYFCARGAYDAYYYYYGMDYWGQGTSVTVSS |
| SEQ ID NO: 87 | EVQLQQSGPELVKPGASVKMSCRASGYTFSTYVMHWIKHRPGQGLEWIGYINPNNDGPNYNEKFKG |
| | KATLTSDISSSTAYMELSSLTSEDSAVYFCSRWDSYNSAAGFAYWGHGTLVTVSA |
| SEQ ID NO: 88 | EVQLQESGGGLVKPGGSLKLSCAASGFTLSTYTMSWVRQTPEKRLEWVAIITSGDTYTYYPDSVKGRF |
| | TISRDNAKNTLYLQMSSLKSEDTGMYYCTRDRPLFHWGQGTTLTVST |
| SEQ ID NO: 89 | EVQLQESGAELVRPGASVKLSCKALGYTFTDYEIHWVKETPVYGLEWIGDIHPGSGGTANNQKFKGK |
| | ATLTADKSSNTAYMELSSLTSEDSAVYYCTRAVSGYYAMDYWGQGTSVTVSS |
| SEQ ID NO: 90 | EVQLQESGAELVRPGTSVKMSCKAAGYTFSNYLIGWIKQRPGHGLEWIGDIYPGDNNTNYNEKFRVK |
| | ATLTADTSSNTAYMHLTSLTSEDSAIYYCAGGTDYDGFANWGQGTLVTVSA |
| SEQ ID NO: 91 | QVQLVQSGAEVKKPGASVKVSCKASGYSFTDYFLNWVRQAPGQGLEWMGRINPYNGDSFINQNFRD |
| | RVTMTRDTSTSTVYMELSSLRSEDTAVYYCARGGYDGYFIAYFDYWGAGTTVTVSS |
| SEQ ID NO: 92 | QVQLVQSGAEVKKPGSSVKVSCKASGYSFTDYFLNWVRQAPGQGLEWMGRINPYNGDSFINQNFRD |
| | RVTITADKSTSTAYMELSSLRSEDTAVYYCARGGYDGYFIAYFDYWGAGTTVTVSS |
| SEQ ID NO: 93 | EVQLVQSGAEVKKPGESLKISCKGSGYSFTDYFLNWVRQMPGKGLEWMGRINPYNGDSFINQNFRDQ |
| | VTISADKSISTAYLQWSSLKASDTAMYYCARGGYDGYFIAYFDYWGAGTTVTVSS |
| SEQ ID NO: 94 | QVQLVQSGAEVKKPGASVKVSCKASGYSFTDYFLNWVRQAPGQGLEWMGRINPYNGDSFINQNFRD |
| | RVTMTVDTSTSTVYMELSSLRSEDTAVYYCARGGYDGYFIAYFDYWGAGTTVTVSS |
| SEQ ID NO: 95 | QVQLVQSGAEVKKPGASVKVSCKASGYTFTDYEIHWVRQAPGQGLEWMGDIHPGSGGTANNQKFK |
| | GRVTMTRDTSTSTVYMELSSLRSEDTAVYYCARAVSGYYAMDYWGQGTLVTVSS |
| SEQ ID NO: 96 | QVQLVQSGAEVKKPGSSVKVSCKASGYTFTDYEIHWVRQAPGQGLEWMGDIHPGSGGTANNQKFKG |
| | RVTITADESTSTAYMELSSLRSEDTAVYYCARAVSGYYAMDYWGQGTLVTVSS |
| SEQ ID NO: 97 | QVQLVQSGAEVKKPGASVKVSCKASGYTFTDYEIHWVRQAPGQGLEWMGDIHPGSGGTANNQKFK |
| | GRVTMTADTSTSTVYMELSSLRSEDTAVYYCTRAVSGYYAMDYWGQGTLVTVSS |

Murine Light Chain (LC) Sequences and Human Light Chain (LC) Sequences

| | |
|---|---|
| SEQ ID NO: 98 | ENVLTQSPAIMSASLGEKVTMSCRASSGVNYMYWYQQKSDASPKLLIYYTSILAPGVPARFSGSGSG |
| | NSYSLTISSMEGEDAATYYCQQFTSSPYTFGGGTKLEIKRADAAPTVSIFPPSSEQLTSGGASVVCFLN |
| | NFYPKDINVKWKIDGSERQNGVLNSWTDQDSKDSTYSMSSTLTLTKDEYERHNSYTCEATHKTSTS |
| | PIVKSFNRNEC |
| SEQ ID NO: 99 | DILLTQSPAILSVSPGERVSFSCRASQSIGTSIHWYQQRTNGSPRLLIKYGSESISGIPSRFSGSGSGTDFT |
| | LSINSVESEDIADYYCQQSNTWPLTFGDGTKLELKRADAAPTVSIFPPSSEQLTSGGASVVCFLNNFYP |
| | KDINVKWKIDGSERQNGVLNSWTDQDSKDSTYSMSSTLTLTKDEYERHNSYTCEATHKTSTSPIVKS |
| | FNRNEC |
| SEQ ID NO: 100 | EIVLTQSPTTMAASPGEKITIICSASSIIGSDFLHWYQQRPGFSPKFLIYRTSILASGVPTRFTGSGSGTSY |
| | SLTIGTMEAEDVATYYCQQGSGLPLTFGSGTKLEMKRADAAPTVSIFPPSSEQLTSGGASVVCFLNNF |
| | YPKDINVKWKIDGSERQNGVLNSWTDQDSKDSTYSMSSTLTLTKDEYERHNSYTCEATHKTSTSPIV |
| | KSFNRNEC |
| SEQ ID NO: 101 | DIQMTQSPSSLSASVGDRVTITCKASQDINSHLSWYQQKPGKAPKLLIYRANRLATGVPSRFSGSGSG |
| | TDFTFTISSLQPEDIATYYCLQYDEFPYTFGGGTKLEIKRTVAAPSVFIFPPSDEQLKSGTASVVCLLNN |
| | FYPREAKVQWKVDNALQSGNSQESVTEQDSKDSTYSLSSTLTLSKADYEKHKVYACEVTHQGLSSP |
| | VTKSFNRGEC |
| SEQ ID NO: 102 | DIQMTQSPSSLSASVGDRVTITCKASQDINSHLSWYQQKPGKAPKLLIYRANRLATGVPSRFSGSGSG |
| | TDFTFTISSLEYEDIATYYCLQYDEFPYTFGGGTKLEIKRTVAAPSVFIFPPSDEQLKSGTASVVCLLNN |
| | FYPREAKVQWKVDNALQSGNSQESVTEQDSKDSTYSLSSTLTLSKADYEKHKVYACEVTHQGLSSP |
| | VTKSFNRGEC |
| SEQ ID NO: 103 | DIQMTQSPSSLSASVGDRVTITCKASQDINSHLSWYQQKPGKAPKLLIYRANRLATGVPSRFSGSGSG |
| | TDFTFTISSLQPEDIATYYCQQYDEFPYTFGGGTKLEIKRTVAAPSVFIFPPSDEQLKSGTASVVCLLN |
| | NFYPREAKVQWKVDNALQSGNSQESVTEQDSKDSTYSLSSTLTLSKADYEKHKVYACEVTHQGLSS |
| | PVTKSFNRGEC |
| SEQ ID NO: 104 | DIKMTQSPSSMYASLGQRVTITCKASQDINSHLSWFQEKPGKSPKTLIYRANRLADGVPSRFSGSGSG |
| | QDYFLTISSLEYEDVGIYYCLQYDEFPYTFGGGTKLEIKRTVAAPSVFIFPPSDEQLKSGTASVVCLLN |
| | NFYPREAKVQWKVDNALQSGNSQESVTEQDSKDSTYSLSSTLTLSKADYEKHKVYACEVTHQGLSS |
| | PVTKSFNRGEC |
| SEQ ID NO: 105 | DIQMTQSPSSLSASVGDRVTITCKASEDIYDRVAWYQQKPGKAPKLLIYGTASLETGVPSRFSGSGSG |
| | TDFTFTISSLQPEDIATYYCQQYWTTPWTFGGGTKVEIKRTVAAPSVFIFPPSDEQLKSGTASVVCLLN |
| | NFYPREAKVQWKVDNALQSGNSQESVTEQDSKDSTYSLSSTLTLSKADYEKHKVYACEVTHQGLSS |
| | PVTKSFNRGEC |
| SEQ ID NO: 106 | DIQMTQSPSSLSASVGDRVTITCKASEDIYDRVAWYQQKPGKAPKLLIYGTASLETGVPSRFSGSGSG |
| | TDFTLTISSLQPEDFATYYCQQYWTTPWTFGGGTKVEIKRTVAAPSVFIFPPSDEQLKSGTASVVCLL |
| | NNFYPREAKVQWKVDNALQSGNSQESVTEQDSKDSTYSLSSTLTLSKADYEKHKVYACEVTHQGL |
| | SSPVTKSFNRGEC |
| SEQ ID NO: 107 | DIQMTQSPSSLSASVGDRVTITCKASEDIYDRVAWYQQKPGKAPKLLIYGTASLETGVLSRFSGSGSG |
| | TDFTLTISSLQAEDFATYYCQQYWTTPWTFGGGTKVEIKRTVAAPSVFIFPPSDEQLKSGTASVVCLL |
| | NNFYPREAKVQWKVDNALQSGNSQESVTEQDSKDSTYSLSSTLTLSKADYEKHKVYACEVTHQGL |
| | SSPVTKSFNRGEC |
| SEQ ID NO: 108 | DIQMTQSSSSFSGSLGDRLTINCKASEDIYDRVAWYQQKPGNAPRLLISGTASLETGVLSRFSGSGSG |
| | KDYTLSINGLQAEDVATYYCQQYWTTPWTFGGGTKLEIKRTVAAPSVFIFPPSDEQLKSGTASVVCL |
| | LNNFYPREAKVQWKVDNALQSGNSQESVTEQDSKDSTYSLSSTLTLSKADYEKHKVYACEVTHQG |
| | LSSPVTKSFNRGEC |

Murine Heavy Chain (HC) Sequences and Human Heavy Chain (HC) Sequences

| | |
|---|---|
| SEQ ID | QVQLQQSGAELMKPGASVKISCKATGYSFTKYWIEWVKQRPGHGLEWIGEILPGSVITNYNEKFKGK |
| NO: 109 | ATFTADTSSNTVYMQLSSLTSEDSAVYYCTKWGLYDSDDGVDYWGQGTTLTVSSAKTTPPSVYPLA |
| | PGSAAQTNSMVTLGCLVKGYFPEPVTVTWNSGSLSSGVHTFPAVLQSDLYTLSSSVTVPSSTWPSETV |
| | TCNVAHPASSTKVDKKIVPRDCGCKPCICTVPEVSSVFIFPPKPKDVLTITLTPKVTCVVVDISKDDPEV |
| | QFSWFVDDVEVHTAQTQPREEQFNSTFRSVSELPIMHQDWLNGKEFKCRVNSAAFPAPIEKTISKTKG |
| | RPKAPQVYTIPPPKEQMAKDKVSLTCMITDFFPEDITVEWQWNGQPAENYKNTQPIMDTDGSYFVYS |
| | KLNVQKSNWEAGNTFTCSVLHEGLHNHHTEKSLSHSPGK |
| SEQ ID | EVKLVESGGGLVQPGGSLKLSCAASGFSFSGCTMSWIRQTPERRLEWVAYISNGGDITYYPDTVKGRF |
| NO: 110 | TISRDNAKNSLYLQMSSLKSEDTAMYYCARLDGYYYAMDFWGQGTSVTVSSAKTTPPSVYPLAPGS |
| | AAQTNSMVTLGCLVKGYFPEPVTVTWNSGSLSSGVHTFPAVLQSDLYTLSSSVTVPSSTWPSETVTCN |
| | VAHPASSTKVDKKIVPRDCGCKPCICTVPEVSSVFIFPPKPKDVLTITLTPKVTCVVVDISKDDPEVQFS |
| | WFVDDVEVHTAQTQPREEQFNSTFRSVSELPIMHQDWLNGKEFKCRVNSAAFPAPIEKTISKTKGRPK |
| | APQVYTIPPPKEQMAKDKVSLTCMITDFFPEDITVEWQWNGQPAENYKNTQPIMDTDGSYFVYSKLN |
| | VQKSNWEAGNTFTCSVLHEGLHNHHTEKSLSHSPGK |
| SEQ ID | EVQLQQSGPEVVKPGASVKMSCKASGYTFTSYVMHWVKQKPGQGLEWIGYINPYNDGPKYNEKFK |
| NO: 111 | GKATLTSDKSSSTAYMELSSLTSEDSAVYFCARWDYFNSASGFAFWGQGTLVTVSAAKTTPPSVYPL |
| | APGSAAQTNSMVTLGCLVKGYFPEPVTVTWNSGSLSSGVHTFPAVLQSDLYTLSSSVTVPSSTWPSET |
| | VTCNVAHPASSTKVDKKIVPRDCGCKPCICTVPEVSSVFIFPPKPKDVLTITLTPKVTCVVVDISKDDPE |
| | VQFSWFVDDVEVHTAQTQPREEQFNSTFRSVSELPIMHQDWLNGKEFKCRVNSAAFPAPIEKTISKTK |
| | GRPKAPQVYTIPPPKEQMAKDKVSLTCMITDFFPEDITVEWQWNGQPAENYKNTQPIMDTDGSYFVY |
| | SKLNVQKSNWEAGNTFTCSVLHEGLHNHHTEKSLSHSPGK |
| SEQ ID | QVQLVQSGAEVKKPGASVKVSCKASGYSFTDYFLNWVRQAPGQGLEWMGRINPYNGDSFINQNFRD |
| NO: 112 | RVTMTRDTSTSTVYMELSSLRSEDTAVYYCARGGYDGYFIAYFDYWGAGTTVTVSSASTKGPSVFPL |
| | APCSRSTSESTAALGCLVKDYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNGQPENNYKTTPPVLDSDG |
| | SFFLYSRLTVDKSRWQEGNVFSCSVMHEALHNHYTQKSLSLSLGK |
| SEQ ID | QVQLVQSGAEVKKPGSSVKVSCKASGYSFTDYFLNWVRQAPGQGLEWMGRINPYNGDSFINQNFRD |
| NO: 113 | RVTITADKSTSTAYMELSSLRSEDTAVYYCARGGYDGYFIAYFDYWGAGTTVTVSSASTKGPSVFPL |
| | APCSRSTSESTAALGCLVKDYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNGQPENNYKTTPPVLDSDG |
| | SFFLYSRLTVDKSRWQEGNVFSCSVMHEALHNHYTQKSLSLSLGK |
| SEQ ID | EVQLVQSGAEVKKPGESLKISCKGSGYSFTDYFLNWVRQMPGKGLEWMGRINPYNGDSFINQNFRD |
| NO: 114 | QVTISADKSISTAYLQWSSLKASDTAMYYCARGGYDGYFIAYFDYWGAGTTVTVSSASTKGPSVFPL |
| | APCSRSTSESTAALGCLVKDYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIA VEWESNGQPENNYKTTPPVLDSDG |
| | SFFLYSRLTVDKSRWQEGNVFSCSVMHEALHNHYTQKSLSLSLGK |
| SEQ ID | QVQLVQSGAEVKKPGASVKVSCKASGYSFTDYFLNWVRQAPGQGLEWMGRINPYNGDSFINQNFRD |
| NO: 115 | RVTMTVDTSTSTVYMELSSLRSEDTAVYYCARGGYDGYFIAYFDYWGAGTTVTVSSASTKGPSVFPL |
| | APCSRSTSESTAALGCLVKDYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNGQPENNYKTTPPVLDSDG |
| | SFFLYSRLTVDKSRWQEGNVFSCSVMHEALHNHYTQKSLSLSLGK |
| SEQ ID | EVQLQQSGPDLVKPGASVKISCKASGYSFTDYFLNWVKQSHGKSLEWIGRINPYNGDSFINQNFRDK |
| NO: 116 | ATLTVDKSSTTAHMDLLSLTSEDSAIYYCGRGGYDGYFIAYFDYWGQGSLVTVSAASTKGPSVFPLA |
| | PCSRSTSESTAALGCLVKDYFPEPVTVSWNSGALTSGVHTFPAVLQSSGLYSLSSVVTVPSSSLGTKTY |
| | TCNVDHKPSNTKVDKRVESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHQDWLNGKEYKCKVSNKGLPSSIEKT |
| | ISKAKGQPREPQVYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSLGK |
| SEQ ID | QVQLVQSGAEVKKPGASVKVSCKASGYTFTDYEIHWVRQAPGQGLEWMGDIHPGSGGTANNQKFK |
| NO: 117 | GRVTMTRDTSTSTVYMELSSLRSEDTAVYYCARAVSGYYAMDYWGQGTLVTVSSASTKGPSVFPLA |
| | PCSRSTSESTAALGCLVKDYFPEPVTVSWNSGALTSGVHTFPAVLQSSGLYSLSSVVTVPSSSLGTKTY |
| | TCNVDHKPSNTKVDKRVESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHQDWLNGKEYKCKVSNKGLPSSIEKT |
| | ISKAKGQPREPQVYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSLGK |
| SEQ ID | QVQLVQSGAEVKKPGSSVKVSCKASGYTFTDYEIHWVRQAPGQGLEWMGDIHPGSGGTANNQKFK |
| NO: 118 | GRVTITADESTSTAYMELSSLRSEDTAVYYCARAVSGYYAMDYWGQGTLVTVSSASTKGPSVFPLAP |
| | CSRSTSESTAALGCLVKDYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMISRTPEVTCVVVDVSQED |
| | PEVQFNWYVDGVEVHNAKTKPREEQFNSTYRVVSVLTVLHQDWLNGKEYKCKVSNKGLPSSIEKTI |
| | SKAKGQPREPQVYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSLGK |
| SEQ ID | QVQLVQSGAEVKKPGASVKVSCKASGYTFTDYEIHWVRQAPGQGLEWMGDIHPGSGGTANNQKFK |
| NO: 119 | GRVTMTADTSTSTVYMELSSLRSEDTAVYYCTRAVSGYYAMDYWGQGTLVTVSSASTKGPSVFPLA |
| | PCSRSTSESTAALGCLVKDYFPEPVTVSWNSGALTSGVHTFPAVLQSSGLYSLSSVVTVPSSSLGTKTY |
| | TCNVDHKPSNTKVDKRVESKYGPPCPPCPAPEFLGGPSVFLFPPKPKDTLMISRTPEVTCVVVDVSQE |
| | DPEVQFNWYVDGVEVHNAKTKPREEQFNSTYRVVSVLTVLHQDWLNGKEYKCKVSNKGLPSSIEKT |
| | ISKAKGQPREPQVYTLPPSQEEMTKNQVSLTCLVKGFYPSDIAVEWESNGQPENNYKTTPPVLDSDGS |
| | FFLYSRLTVDKSRWQEGNVFSCSVMHEALHNHYTQKSLSLSLGK |
| SEQ ID | EVQLQESGAELVRPGASVKLSCKALGYTFTDYEIHWVKETPVYGLEWIGDIHPGSGGTANNQKFKGK |
| NO: 120 | ATLTADKSSNTAYMELSSLTSEDSAVYYCTRAVSGYYAMDYWGQGTSVTVSSASTKGPSVFPLAPCS |
| | RSTSESTAALGCLVKDYFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWESNGQPENNYKTTPPVLDSDGSF |
| | FLYSRLTVDKSRWQEGNVFSCSVMHEALHNHYTQKSLSLSLGK |

SIRPα and SIRPγ Sequences

| | | |
|---|---|---|
| SEQ ID | SIRPα | EEELQVIQPDKSVSVAAGESAILHCTVTSLIPVGPIQWFRGAGPARELIYNQKEGHFPRVTTVS |
| NO: 121 | | ESTKRENMDFSISISNITPADAGTYYCVKFRKGSPDTEFKSGAGTELSVRAKPSAPVVSGPAAR |
| | | ATPQHTVSFTCESHGFSPRDITLKWFKNGNELSDFQTNVDPVGESVSYSIHSTAKVVLTREDV |
| | | HSQVICEVAHVTLQGDPLRGTANLSETIRVPPTLEVTQQPVRAENQVNVTCQVRKFYPQRLQ |
| | | LTWLENGNVSRTETASTVTENKDGTYNWMSWLLVNVSAHRDDVKLTCQVEHDGQPAVSKS |
| | | HDLKVSAHPKEQGSNTAAENTGSNERNIYIVVGVVCTLLVALLMAALYLVRIRQKKAQGSTS |
| | | STRLHEPEKNAREITQDTNDITYADLNLPKGKKPAPQAAEPNNHTEYASIQTSPQPASEDTLT |
| | | YADLDMVHLNRTPKQPAPKPEPSFSEYASVQVPRK |
| SEQ ID | SIRPγ | EEELQMIQPEKLLLVTVGKTATLHCTVTSLLPVGPVLWFRGVGPGRELIYNQKEGHFPRVTT |
| NO: 122 | | VSDLTKRNNMDFSIRISSITPADVGTYYCVKFRKGSPENVEFKSGPGTEMALGAKPSAPVVLG |
| | | PAARTTPEHTVSFTCESHGFSPRDITLKWFKNGNELSDFQTNVDPTGQSVAYSIRSTARVVLD |
| | | PWDVRSQVICEVAHVTLQGDPLRGTANLSEAIRVPPTLEVTQQPMRVGNQVNVTCQVRKFY |
| | | PQSLQLTWSENGNVCQRETASTLTENKDGTYNWTSWFLVNISDQRDDVVLTCQVKHDGQL |
| | | AVSKRLALEVTVHQKDQSSDATPGPASSLTALLLIAVLLGPIYVPWKQKT |

Human IgG Fc Sequences

| | |
|---|---|
| Human Fc IgG1 | ASTKGPSVFPLAPSSKSTSGGTAALGCLVKDYFPEPVTVSWNSGALTSGVHTFPAVLQSSGLY |
| SEQ ID NO: 123 | SLSSVVTVPSSSLGTQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK |
| Human Fc IgG1-N297Q | ASTKGPSVFPLAPSSKSTSGGTAALGCLVKDYFPEPVTVSWNSGALTSGVHTFPAVLQSSGLY |
| SEQ ID NO: 124 | SLSSVVTVPSSSLGTQTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKPREEQYQSTYRVVSV |
| | LTVLHQDWLNGKEYKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK |
| Human Fc-IgG2 | ASTKGPSVFPLAPCSRSTSESTAALGCLVKDYFPEPVTVSWNSGALTSGVHTFPAVLQSSGLY |
| SEQ ID NO: 125 | SLSSVVTVPSSNFGTQTYTCNVDHKPSNTKVDKTVERKCCVECPPCPAPPVAGPSVFLFPPKP |
| | KDTLMISRTPEVTCVVVDVSHEDPEVQFNWYVDGVEVHNAKTKPREEQFNSTFRVVSVLTV |
| | VHQDWLNGKEYKCKVSNKGLPAPIEKTISKTKGQPREPQVYTLPPSREEMTKNQVSLTCLVK |
| | GFYPSDIAVEWESNGQPENNYKTTPPMLDSDGSFFLYSKLTVDKSRWQQGNVFSCSVMHEA |
| | LHNHYTQKSLSLSPGK |
| Human Fc-IgG3 | ASTKGPSVFPLAPCSRSTSGGTAALGCLVKDYFPEPVTVSWNSGALTSGVHTFPAVLQSSGLY |
| SEQ ID NO: 126 | SLSSVVTVPSSSLGTQTYTCNVNHKPSNTKVDKRVELKTPLGDTTHTCPRCPEPKSCDTPPPCP |
| | RCPEPKSCDTPPPCPRCPEPKSCDTPPPCPRCPAPELLGGPSVFLFPPKPKDTLMISRTPEVTCV |
| | VVDVSHEDPEVQFKWYVDGVEVHNAKTKPREEQYNSTFRVVSVLTVLHQDWLNGKEYKC |
| | KVSNKALPAPIEKTISKTKGQPREPQVYTLPPSREEMTKNQVSLTCLVKGFYPSDIAVEWESS |
| | GQPENNYNTTPPMLDSDGSFFLYSKLTVDKSRWQQGNIFSCSVMHEALHNRFTQKSLSLSPG |
| | K |
| Human Fc-IgG4 | ASTKGPSVFPLAPCSRSTSESTAALGCLVKDYFPEPVTVSWNSGALTSGVHTFPAVLQSSGLY |
| SEQ ID NO: 127 | SLSSVVTVPSSSLGTKTYTCNVDHKPSNTKVDKRVESKYGPPCPSCPAPEFLGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLG |
| Human Fc-IgG4 S228P | ASTKGPSVFPLAPCSRSTSESTAALGCLVKDYFPEPVTVSWNSGALTSGVHTFPAVLQSSGLY |
| SEQ ID NO: 128 | SLSSVVTVPSSSLGTKTYTCNVDHKPSNTKVDKRVESKYGPPCPPCPAPEFLGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLG |
| Human Fc-IgG4 PE | ASTKGPSVFPLAPCSRSTSESTAALGCLVKDYFPEPVTVSWNSGALTSGVHTFPAVLQSSGLY |
| SEQ ID NO: 129 | SLSSVVTVPSSSLGTKTYTCNVDHKPSNTKVDKRVESKYGPPCPPCPAPEFEGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLGK |
| Human Fc-IgG4 PE' | ASTKGPSVFPLAPCSRSTSESTAALGCLVKDYFPEPVTVSWNSGALTSGVHTFPAVLQSSGLY |
| SEQ ID NO: 130 | SLSSVVTVPSSSLGTKTYTCNVDHKPSNTKVDKRVESKYGPPCPPCPAPEFEGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLG |
| Human kappa LC | RTVAAPSVFIFPPSDEQLKSGTASVVCLLNNFYPREAKVQWKVDNALQSGNSQESVTEQDSK |
| SEQ ID NO: 131 | DSTYSLSSTLTLSKADYEKHKVYACEVTHQGLSSPVTKSFNRGEC |

### Example 2

### Binding of SIRP Monoclonal Antibodies to SIRPα

The binding of anti-SIRP monoclonal antibodies (mAbs) of the present disclosure to SIRP alpha (SIRPα) was determined by solid phase ELISA using an Fe tagged human SIRP alpha. Binding by soluble anti-SIRP antibodies was measured in vitro.

Fe tagged human SIRPα (ACRO #SIG-H5251, genotype variant 1) is adsorbed to high-binding microtiter plates at a concentration of 1 μg/ml diluted in phosphate buffered saline (PBS) overnight at 4° C. The coating solution is removed, the wells are washed and then blocked with 75% casein in PBS containing 0.5% Tween 20 (PBST) for 60 minutes at room temperature while shaking. Blocking solution is removed, the wells are washed and incubated for 60 minutes at room temperature while shaking with either murine or human anti-SIRP mAbs diluted in PBST at a starting concentration of 30 μg/ml and reducing the concentration in 3-fold serial dilutions. Wells are washed three times with PBST and incubated for 60 minutes at room temperature while shaking with an HRP-labeled donkey anti-mouse or anti-human secondary antibody (Jackson ImmunoResearch Laboratories) diluted 1:10,000 in PBST. The wells are washed and then incubated with peroxidase substrate and the absorbance at 450 nm measured. The apparent affinities were calculated using a non-linear fit model (GraphPad Prism).

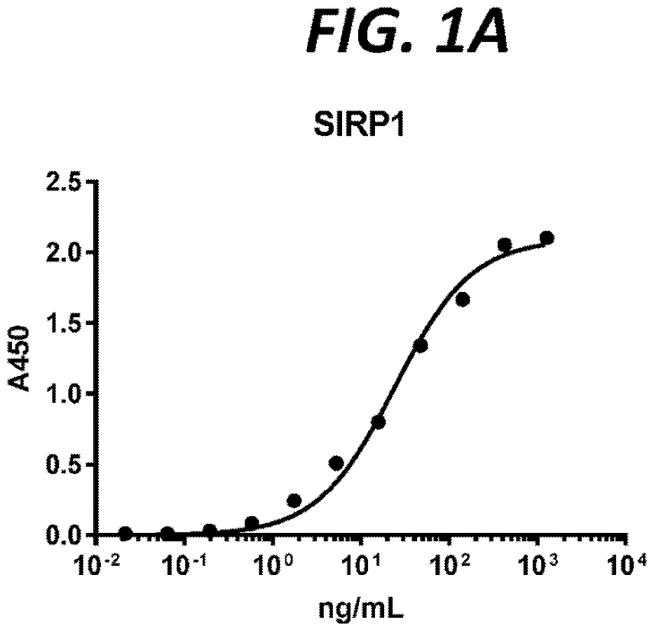

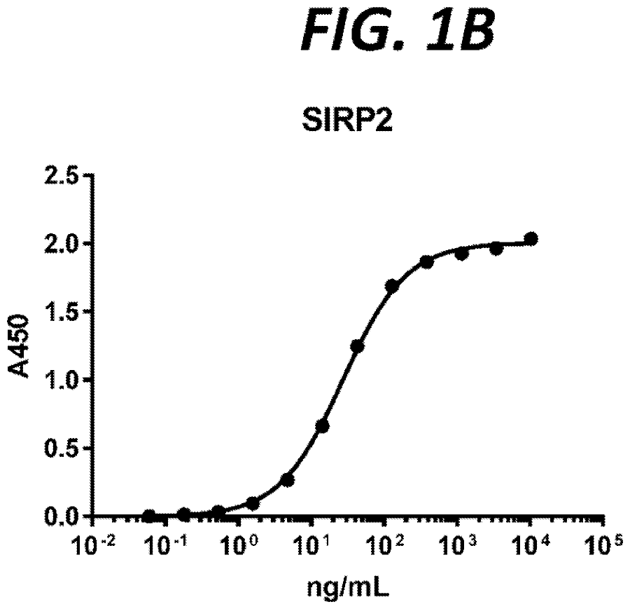

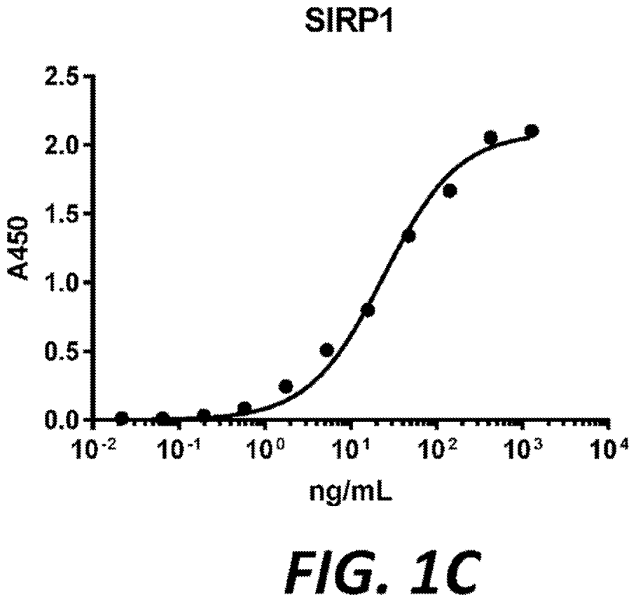

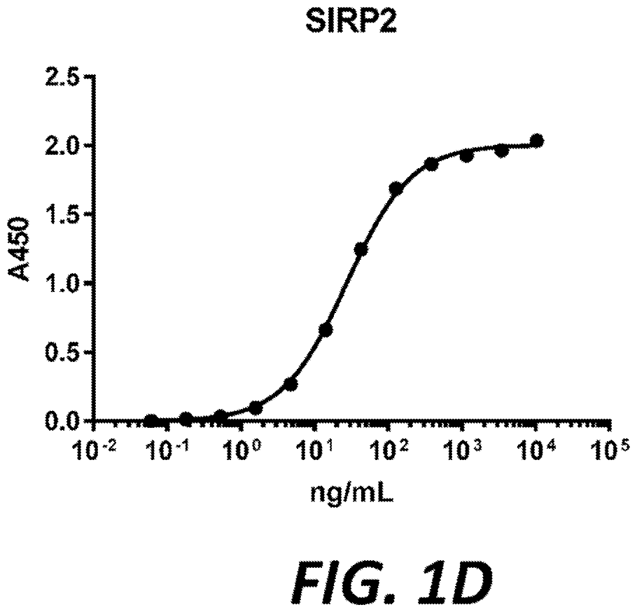

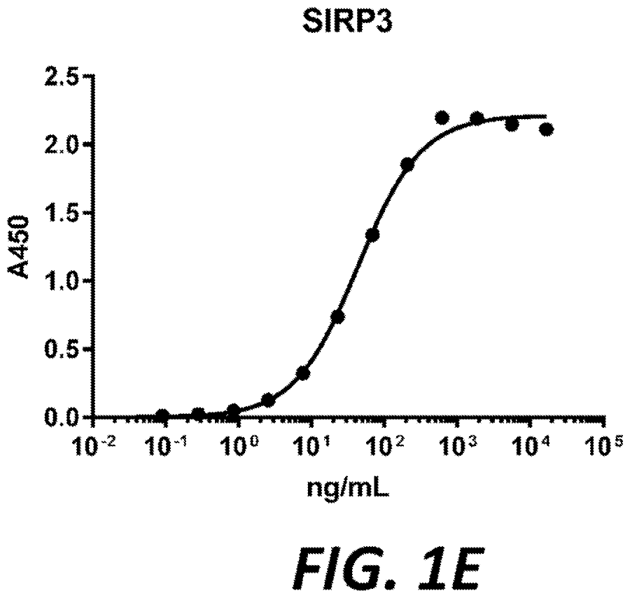

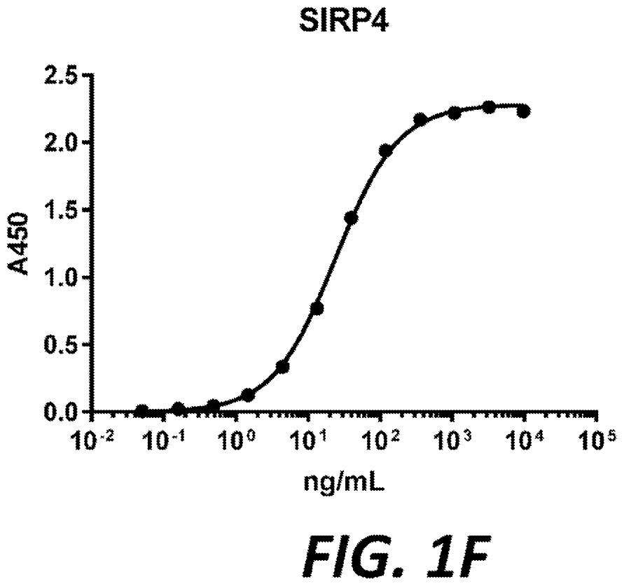

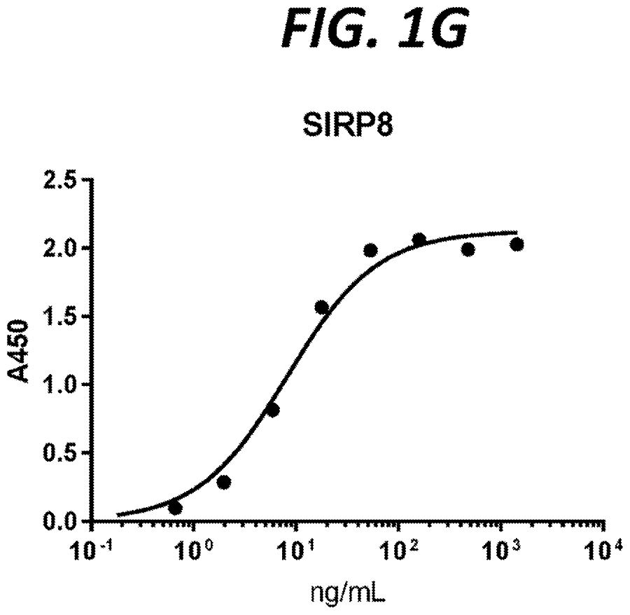

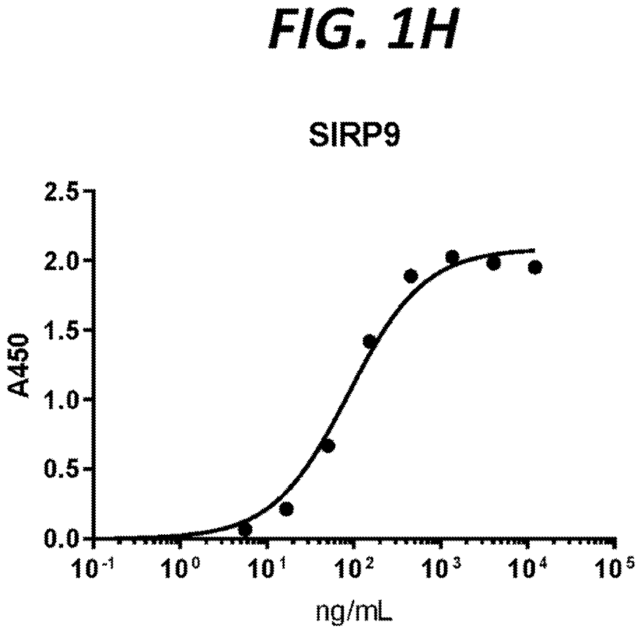

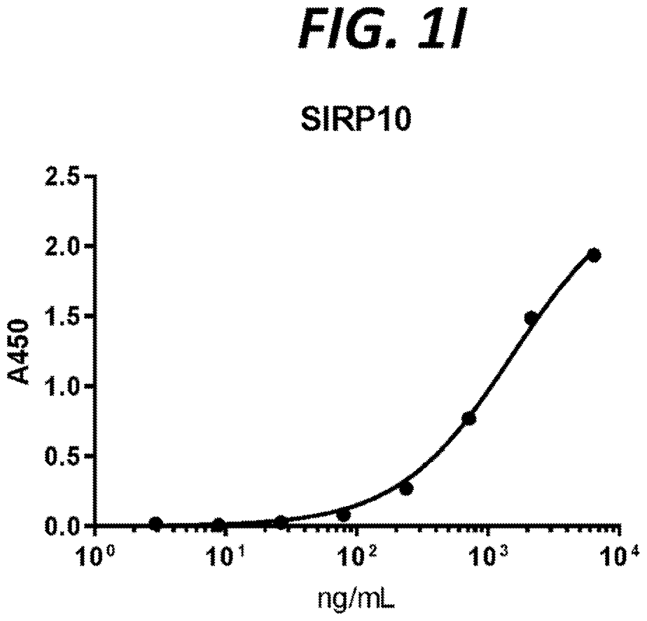

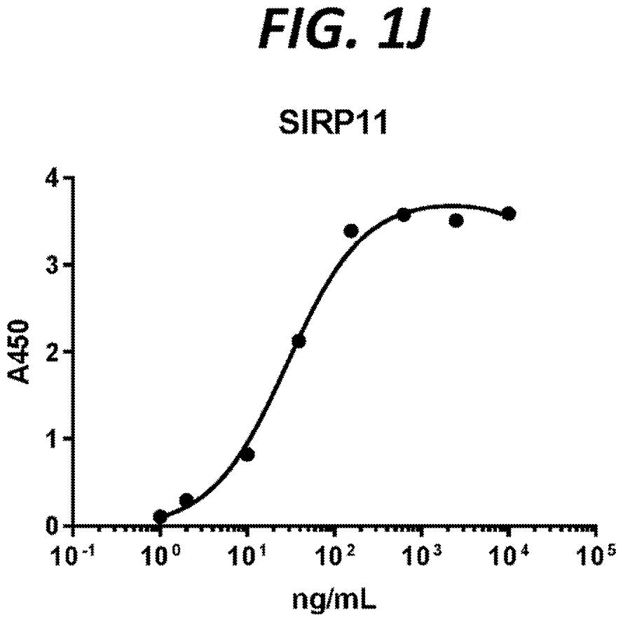

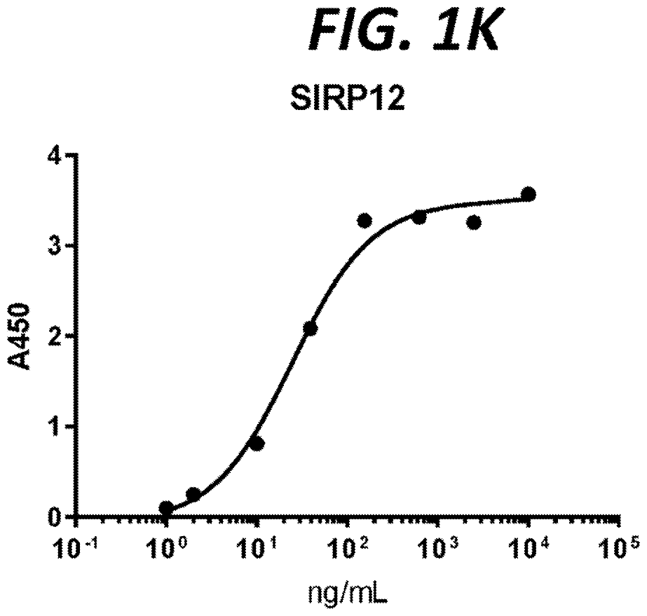

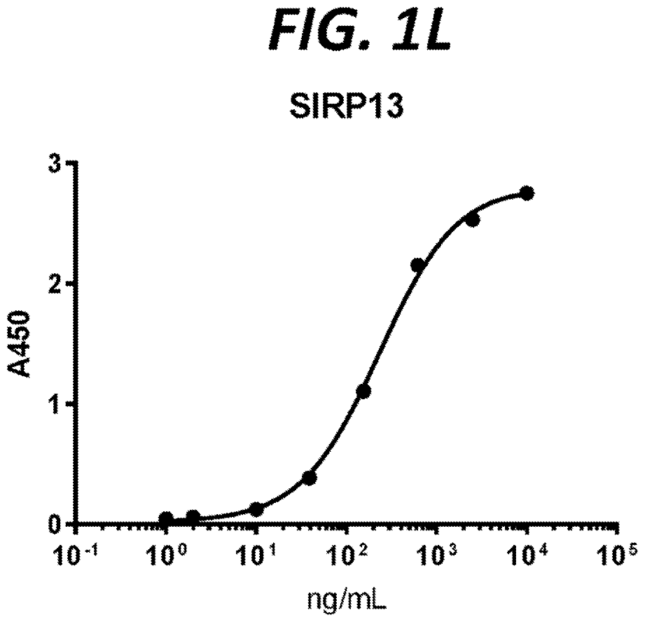

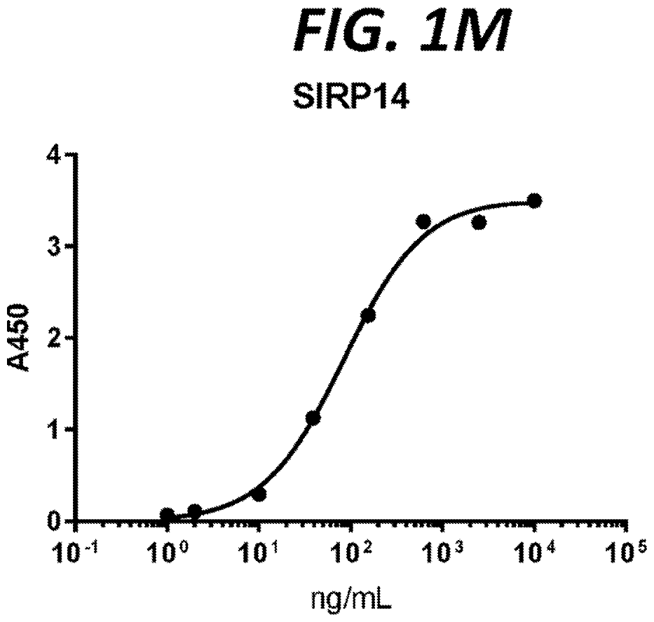

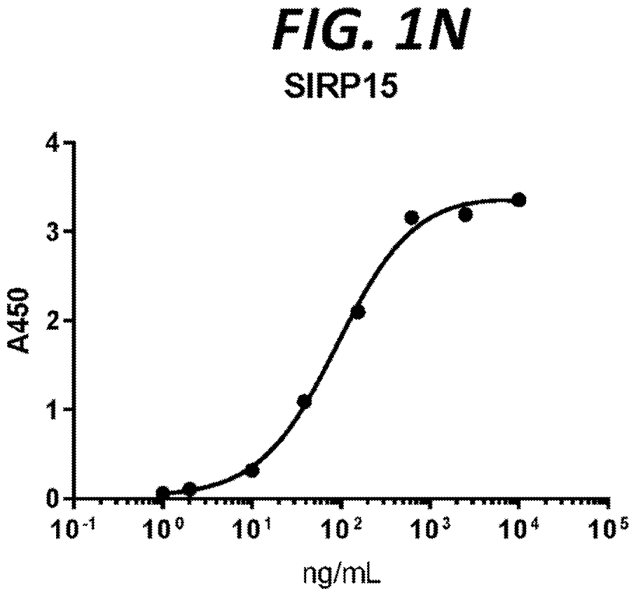

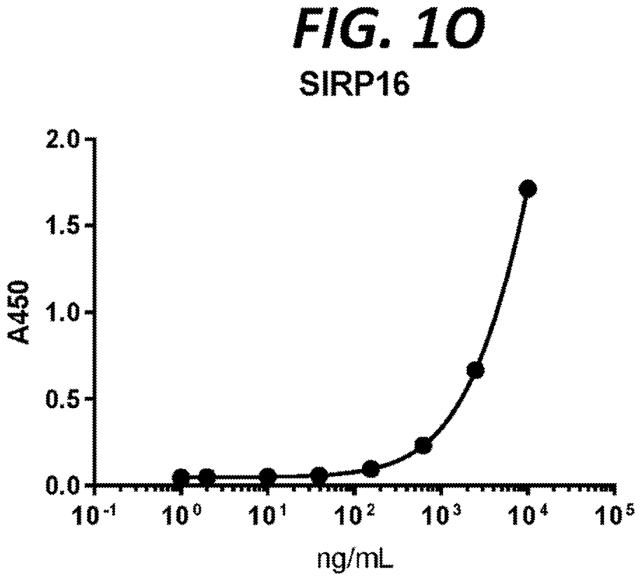

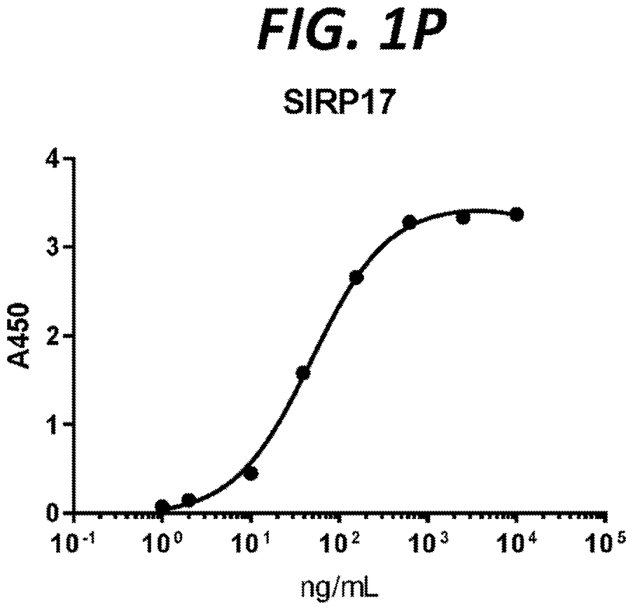

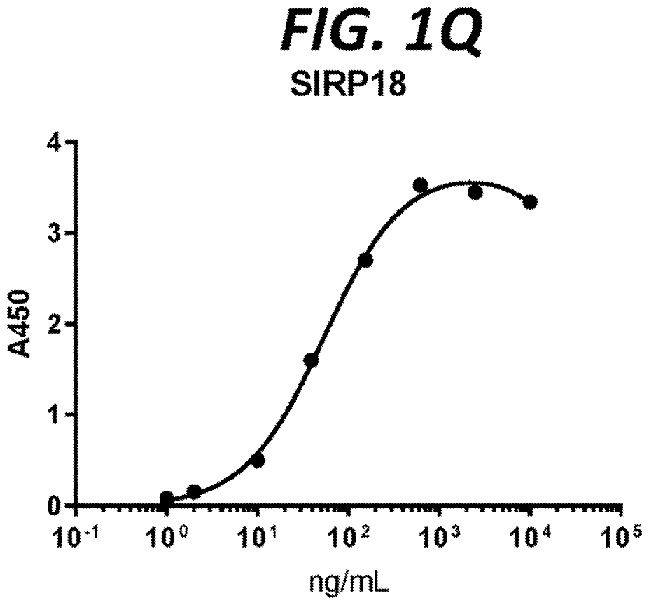

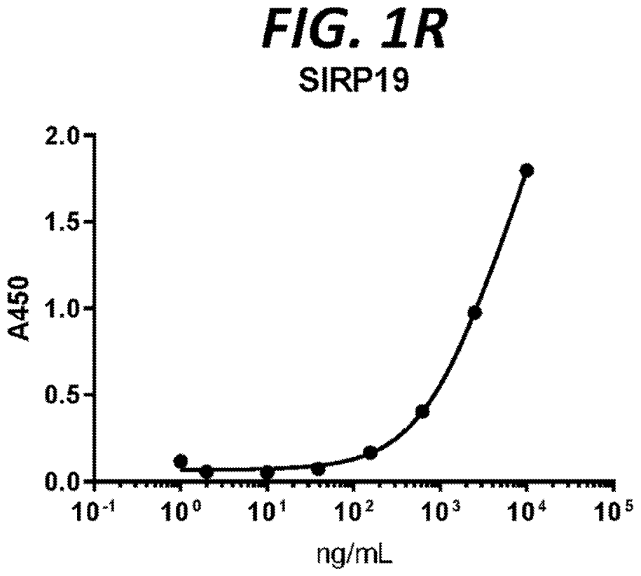

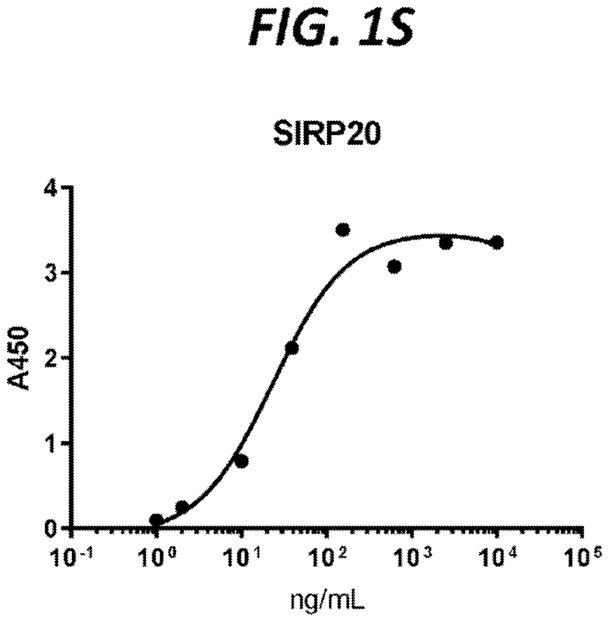

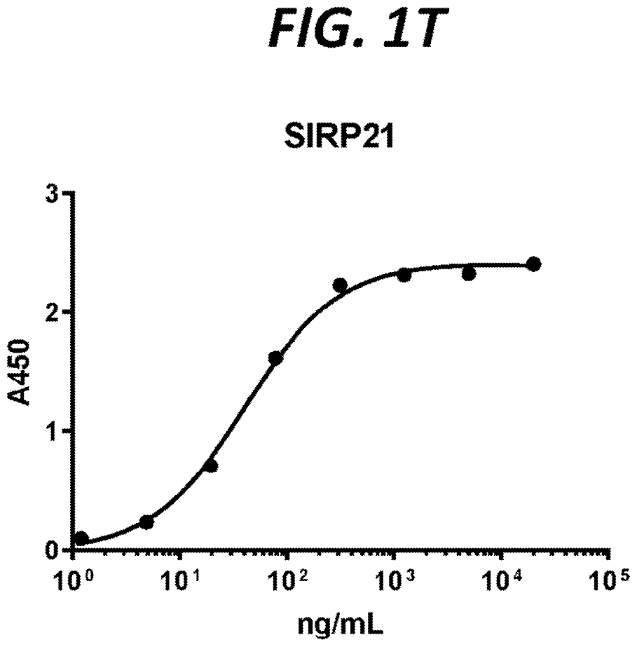

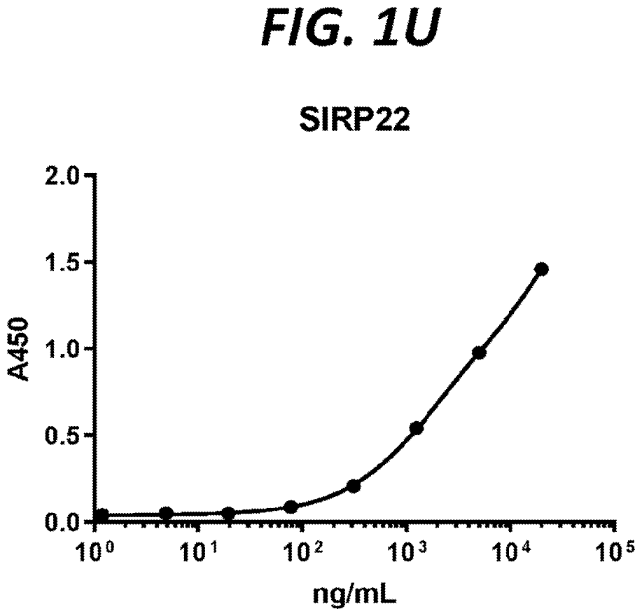

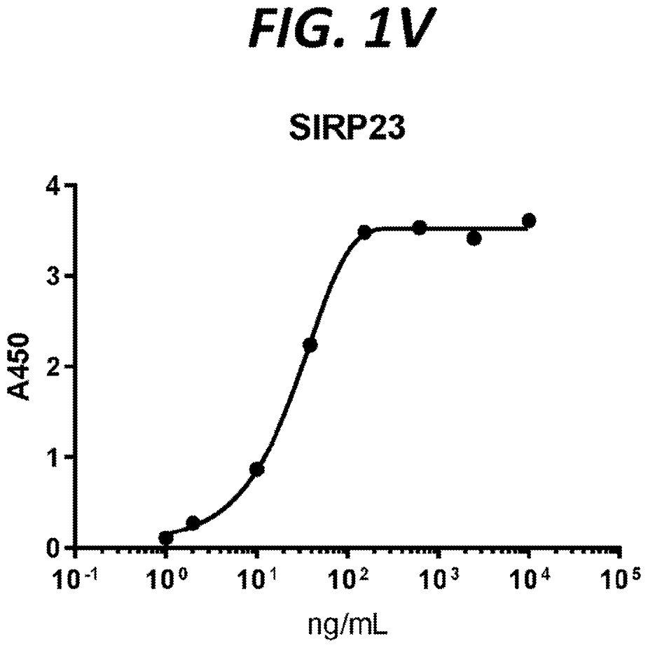

As shown in Table 1, all soluble anti-SIRP mAbs bound to human SIRPα with apparent affinities in the picomolar to nanomolar range. FIG. 1A-FIG. 1V demonstrate representative binding curves for antibodies of the present disclosure.

TABLE 1
Binding of anti-SIRP Antibodies to human SIRPα.

| | Human SIRPα |
|---|---|
| | binding K_{d} (pM) |
| SIRP1 | 39 |
| SIRP2 | 182 |
| SIRP3 | 289 |
| SIRP4 | 161 |
| SIRP5 | 65 |
| SIRP6 | 131 |
| SIRP7 | 197 |
| SIRP8 | 57 |
| SIRP9 | 583 |
| SIRP10 | >10,000 |
| SIRP11 | 194 |
| SIRP12 | 165 |
| SIRP13 | 1,565 |
| SIRP14 | 565 |
| SIRP15 | 608 |
| SIRP16 | >40,000 |
| SIRP17 | 326 |
| SIRP18 | 364 |
| SIRP19 | >19,000 |
| SIRP20 | 157 |
| SIRP21 | 274 |
| SIRP22 | >11,000 |
| SIRP23 | 164 |

### Example 3

### Binding of Mouse Anti-SIRP mAbs to THP-1 Cells Expressing SIRPα

Binding activity of hybridoma-derived mouse SIRP antibodies SIRP1, SIRP2, and SIRP3 to THP-1 cells which express SIRPα, but not SIRPγ, was determined by flow cytometry.

THP-1 cells were incubated for 60 min at 37° C. with increasing concentrations of the mAbs diluted in PBS, pH 7.2. Cells were then washed with PBS and incubated for an additional hour with Alexa Fluor-647 labeled donkey anti-mouse antibody (Jackson ImmunoResearch Laboratories) in PBS. Cells were washed and binding analyzed using a C6 Accuri Flow Cytometer (Becton Dickinson).

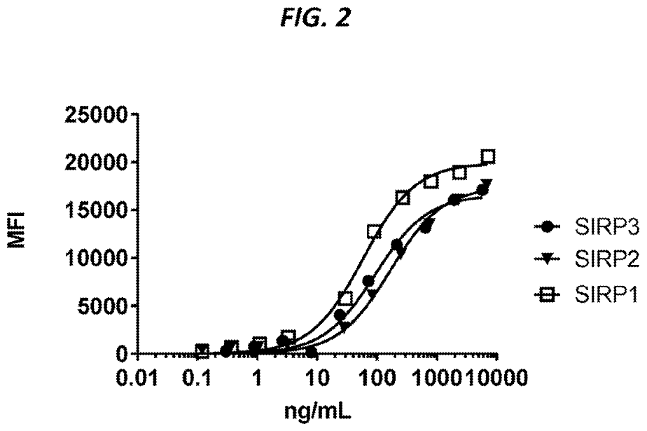

As shown in FIG. 2, all the antibodies bound to SIRPα expressing THP-1 cells in a concentration-dependent manner.

### Example 4

### Binding of SIRP mAbs to SIRPγ

The binding of anti-SIRP antibodies of the present disclosure to SIRP gamma (SIRPγ) was determined by ELISA using an Fc tagged human SIRPγ. Binding by soluble anti-SIRP antibodies was measured in vitro.

Fc tagged human SIRPγ (ACRO #SIG-H5253) is adsorbed to high-binding microtiter plates at a concentration of 1 μg/ml in phosphate buffered saline (PBS) overnight at 4° C. The coating solution is removed, the wells are washed and then blocked with 75% casein in PBS containing 0.5% Tween 20 (PBST) for 60 minutes at room temperature while shaking. Blocking solution is removed, the wells are washed and incubated for 60 minutes at room temperature while shaking with anti-SIRP mAbs diluted in PBST at a starting concentration of 30 μg/ml and reducing the concentration in 3-fold serial dilutions. Wells are washed three times with PBST and incubated for 60 minutes at room temperature while shaking with an HRP labeled donkey anti-mouse or anti-human secondary antibody (Jackson ImmonResearch Laboratories) diluted 1:10,000 in PBST. The wells are washed and then incubated with peroxidase substrate and the absorbance at 450 nm determined. The apparent affinities were calculated using a non-linear fit model (GraphPad Prism).

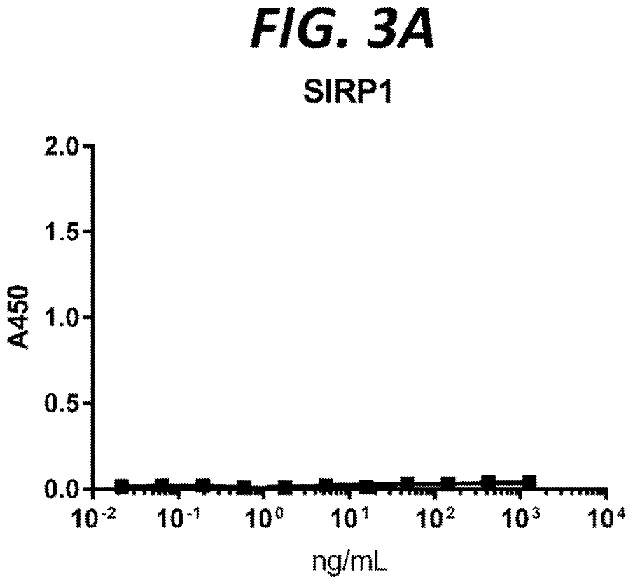

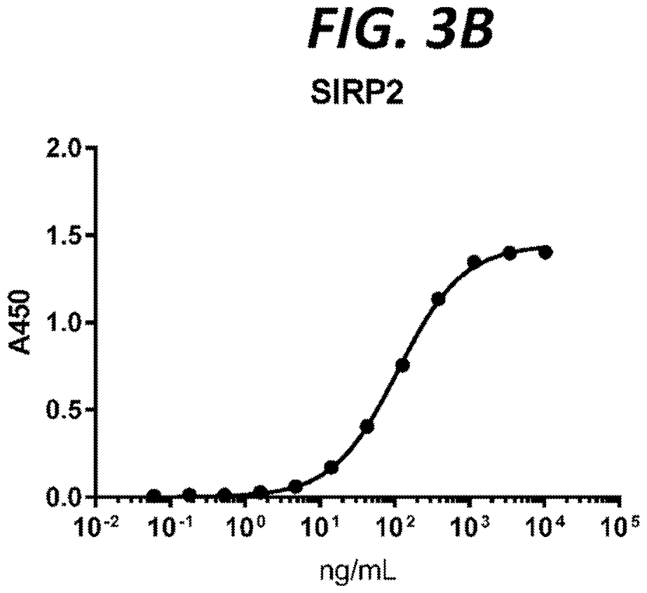

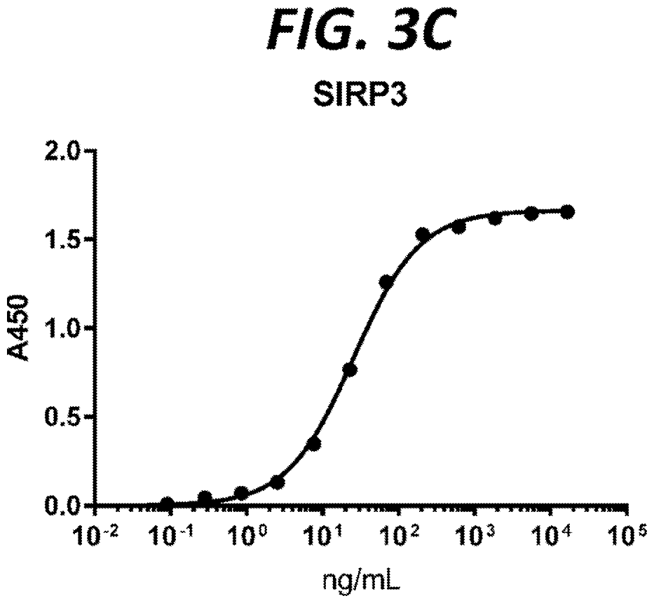

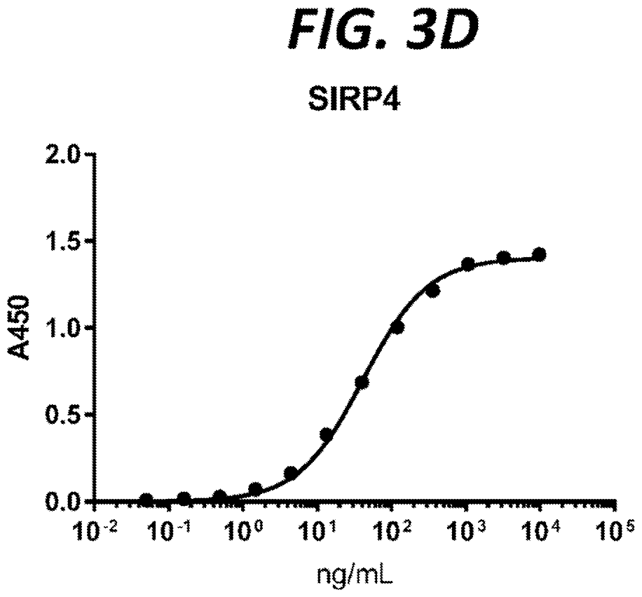

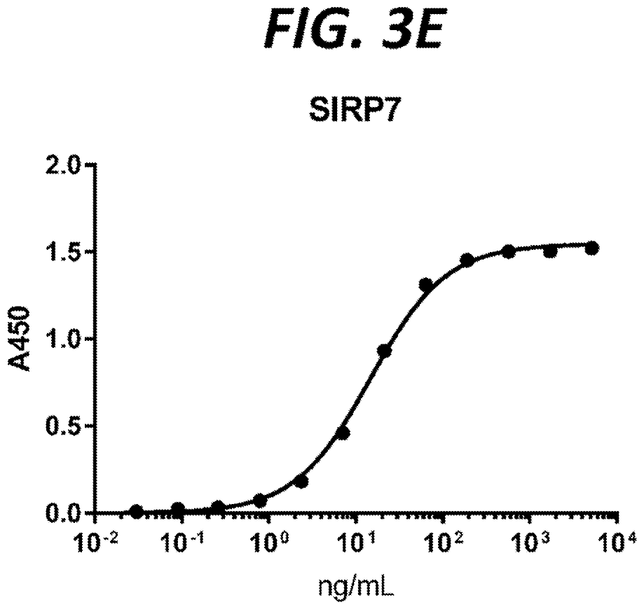

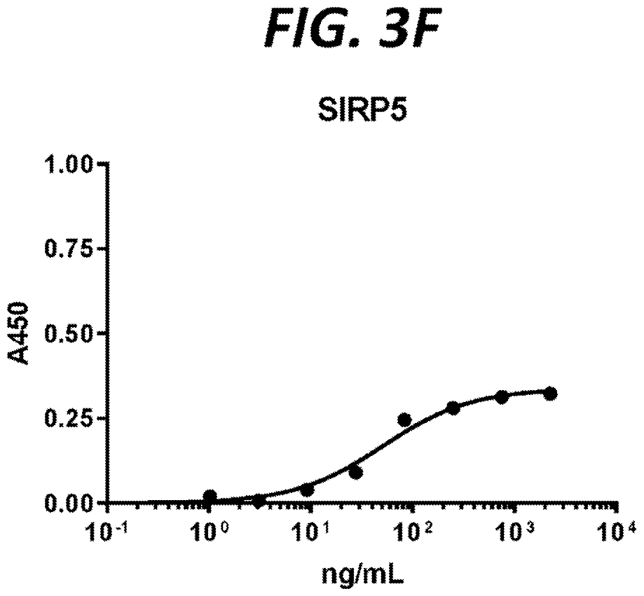

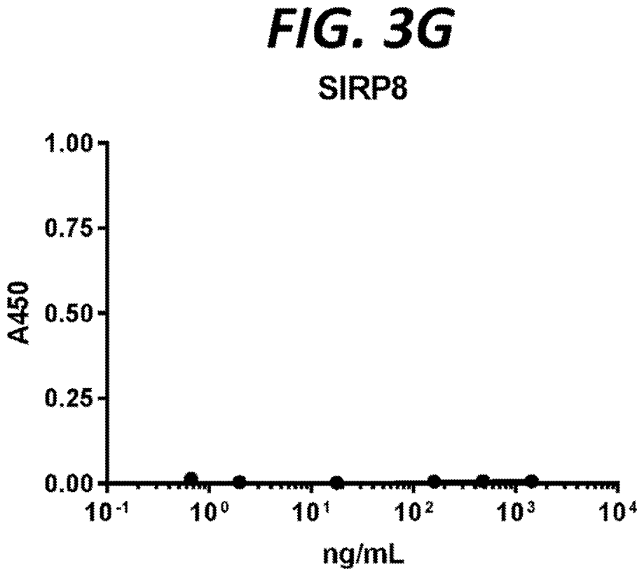

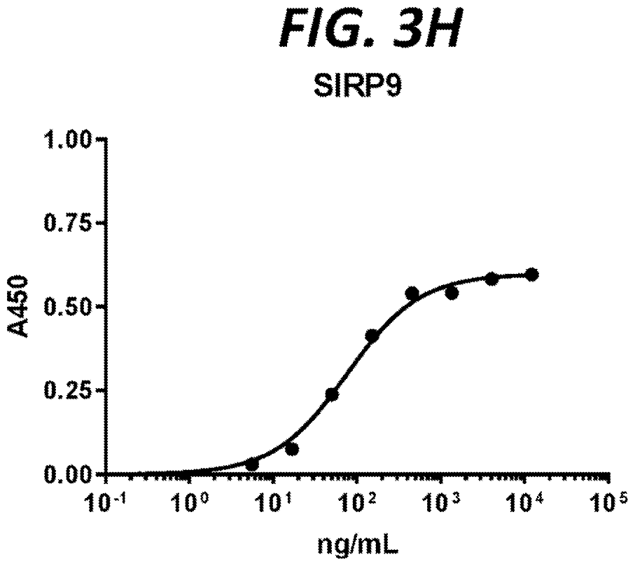

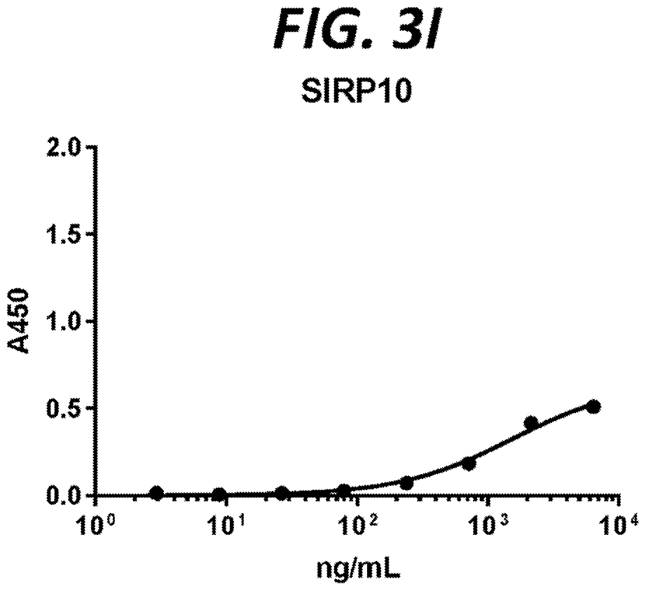

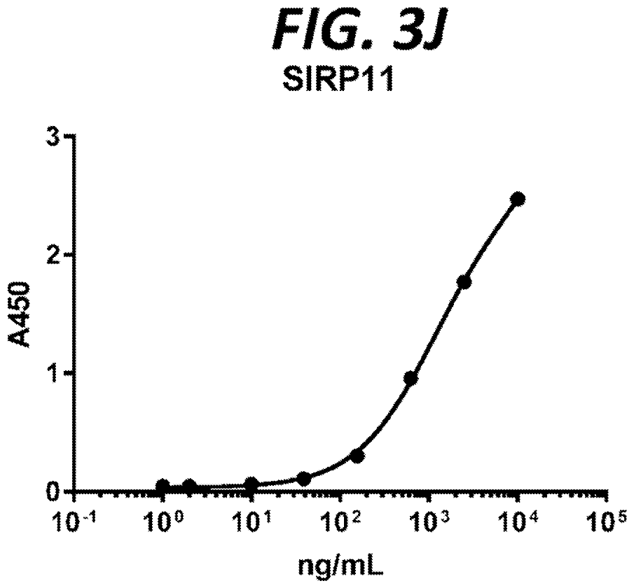

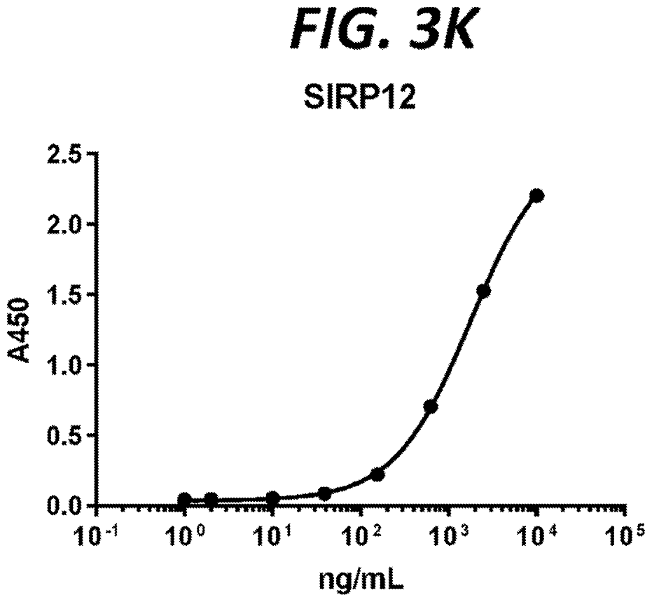

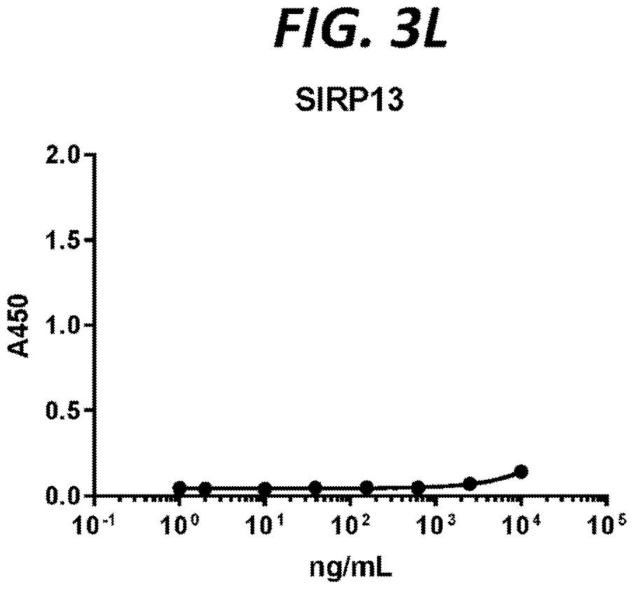

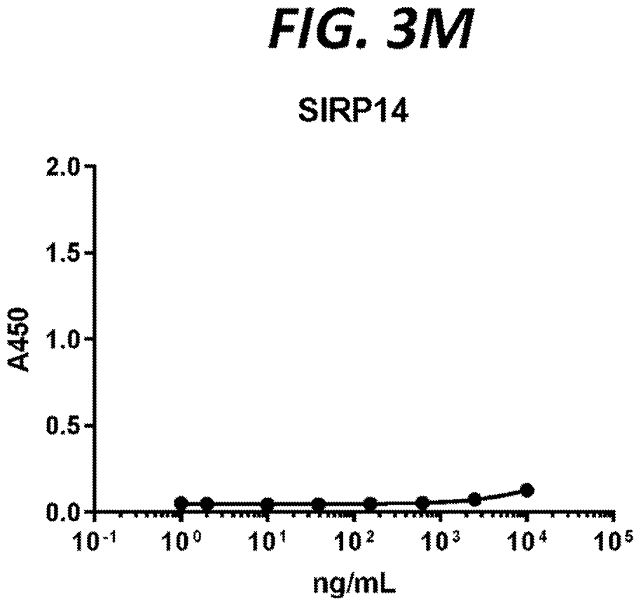

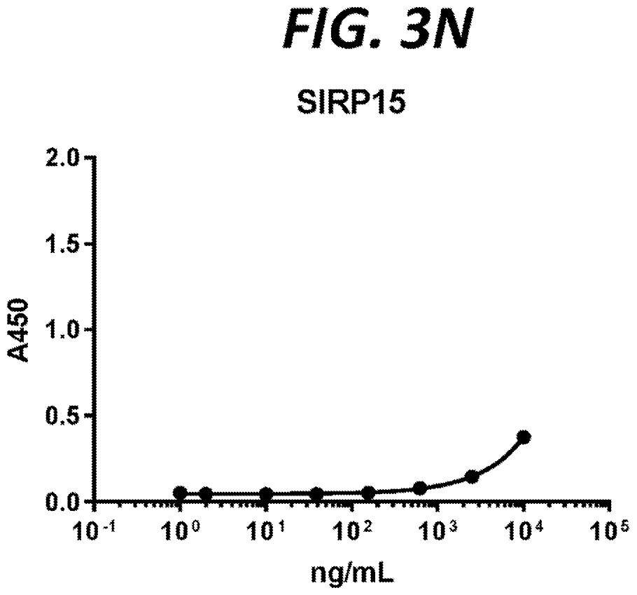

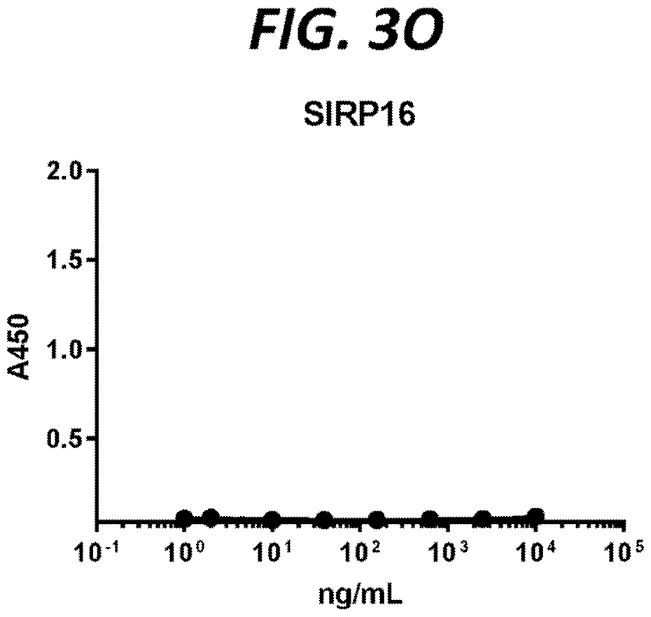

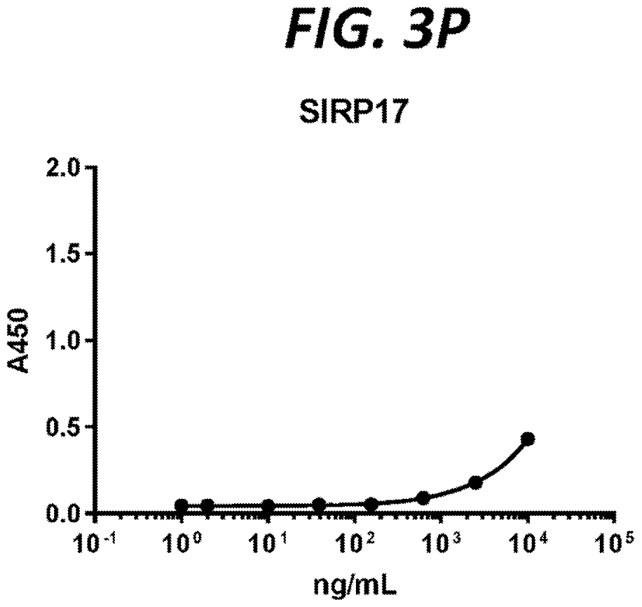

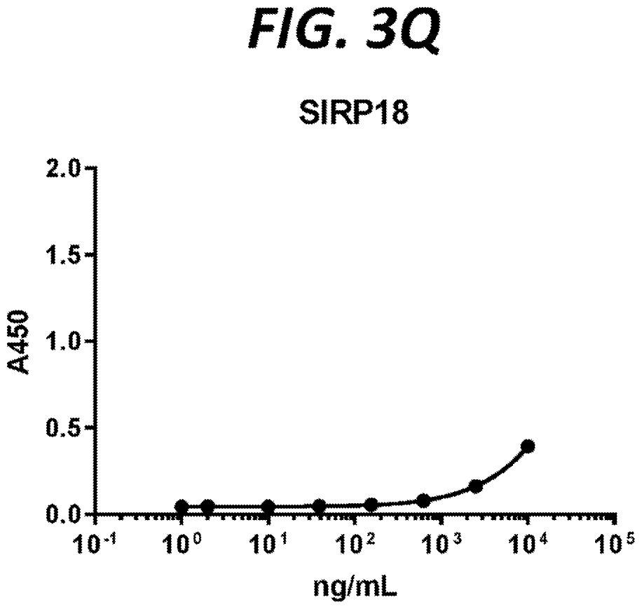

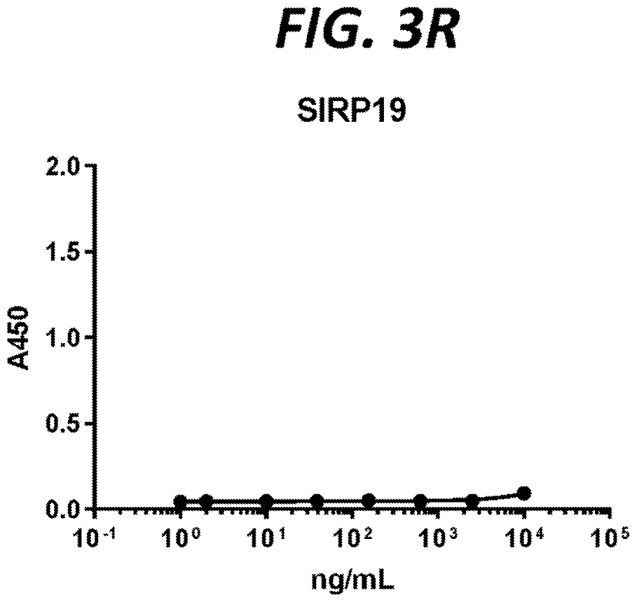

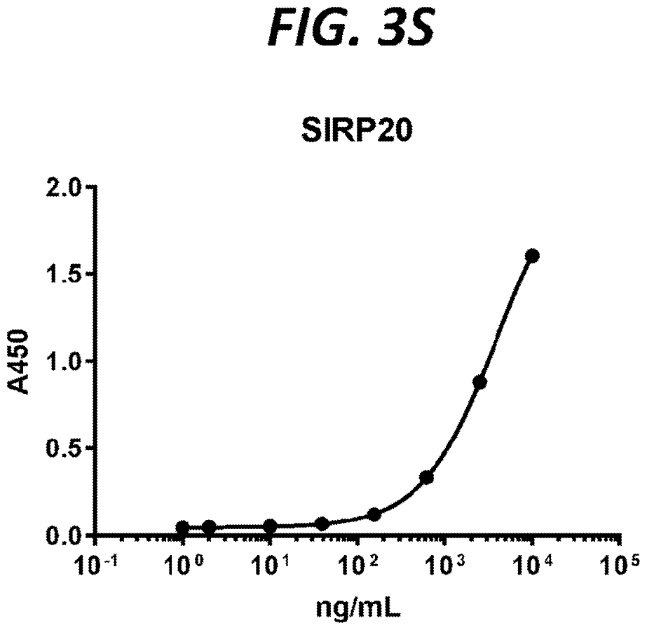

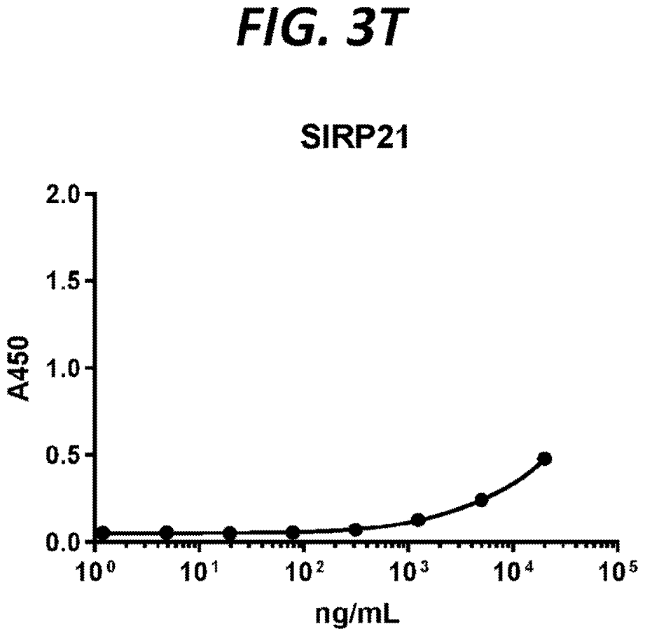

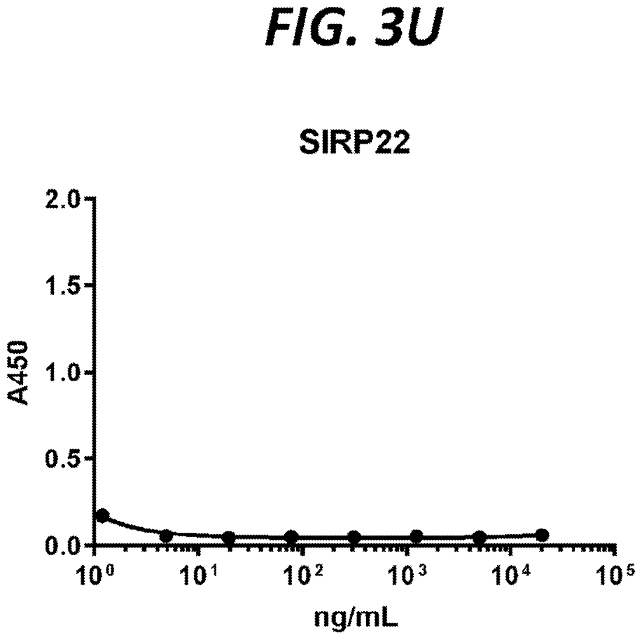

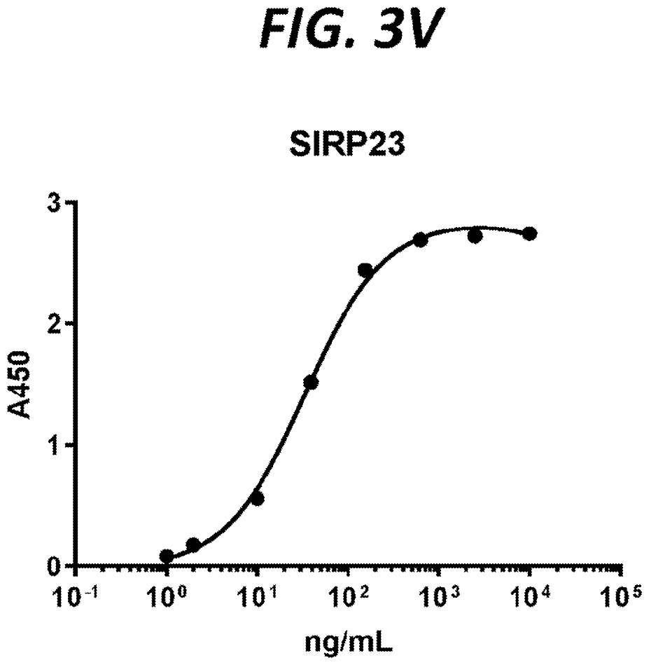

As shown in Table 2, the soluble anti-SIRP mAbs SIRP2, SIRP3, SIRP4, SIRP5, SIRP6, SIRP7, SIRP9, SIRP10, SIRP11, SIRP12, SIRP16, SIRP17, SIRP18, SIRP20, SIRP21 and SIRP23 bound to human SIRP gamma with apparent affinities in the picomolar or nanomolar range. Additionally, the anti-SIRP mAb SIRP1, SIRP8, SIRP13, SIRP14, SIRP15, SIRP19, and SIRP22 did not appreciably bind human SIRP gamma at mAb concentrations up to 30 μg/ml. FIG. 3A-FIG. 3V demonstrate representative binding curves derived from antibodies of the present disclosure.

TABLE 2
Binding of anti-SIRP Antibodies to Human SIRPγ.

| | Human SIRPγ |
|---|---|
| | binding K_{d} (pM) |
| SIRP1 | *NB |
| SIRP2 | 734 |
| SIRP3 | 170 |
| SIRP4 | 274 |
| SIRP5 | 126 |
| SIRP6 | 183 |
| SIRP7 | 99 |
| SIRP8 | *NB |
| SIRP9 | 510 |
| SIRP10 | >10,000 |
| SIRP11 | 7,223 |
| SIRP12 | >12,000 |
| SIRP13 | *NB |
| SIRP14 | *NB |
| SIRP15 | >14,000 |
| SIRP16 | *NB |
| SIRP17 | >15,000 |
| SIRP18 | >34,000 |
| SIRP19 | *NB |
| SIRP20 | >29,000 |
| SIRP21 | >21,000 |
| SIRP22 | *NB |
| SIRP23 | 225 |

*NB—no binding detected at mAb concentration up to 30 μg/ml

### Example 5

### Binding of Mouse mAbs to Jurkat T Cells Expressing SIRPγ

Binding activity of mouse hybridoma-derived SIRP mAbs to Jurkat cells which express SIRPγ, but not SIRPα, was determined by flow cytometry.

Jurkat cells were incubated for 60 min at 37° C., 5% CO₂ with increasing concentrations of the anti-SIRP mAbs diluted in phosphate buffered saline (PBS), pH 7.2. Cells were then washed with PBS and incubated for an additional hour with Alexa Fluor-647 labeled donkey anti-mouse antibody (Jackson ImmunoResearch Laboratories) in PBS. Cells were washed and binding analyzed using a C6 Accuri Flow Cytometer (Becton Dickinson). Alternatively, the cells were incubated for 1 h at 37° C. with the saturating concentration of 10 μg/ml of SIRP mAbs in binding buffer containing 1 mM EDTA (Sigma Aldrich), 1% FBS (Biowest) in PBS (Corning). The cells were then washed and stained for 45 min under the same conditions with donkey anti-mouse IgG fluorescein isothiocyanate (FITC)-linked secondary antibody (Jackson ImmunoResearch Laboratories). The cells were then washed and analyzed by flow cytometry (Attune, Life Technologies).

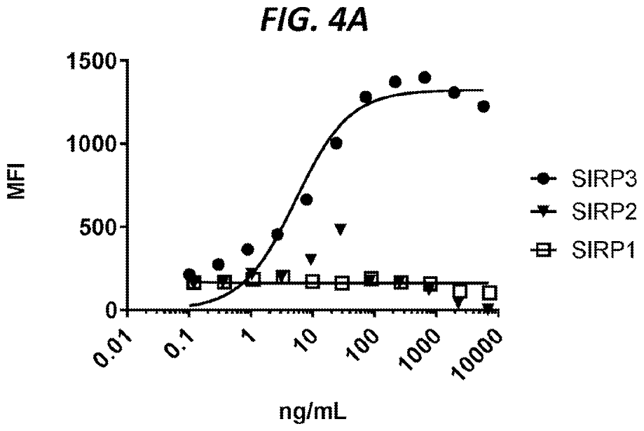

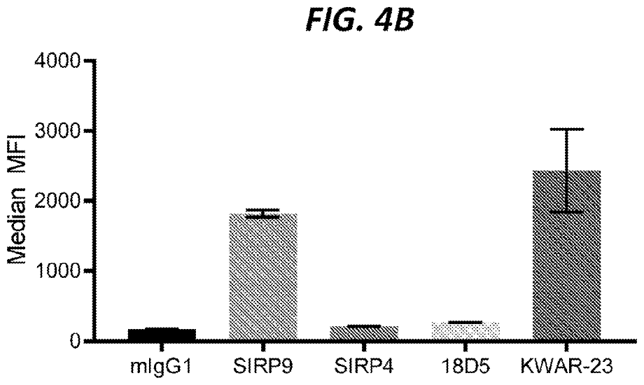

As shown in FIG. 4A, SIRP3 bound to SIRPγ expressing Jurkat cells whereas SIRP2 or SIRP1 exhibited no binding. In addition, as shown in FIG. 4B, SIRP9 bound to Jurkat cells at a concentration of 10 μg/ml, comparable to KWAR-23 which has previously been shown to bind to SIRPγ whereas SIRP4 exhibited no binding to SIRPγ on the Jurkat cells.

### Example 6

### Anti-SIRP mAbs Block CD47/SIRPα Binding

To assess the ability of anti-SIRP antibodies of the present disclosure to block the binding of CD47 to SIRPα in vitro the following method was employed using ELISA plates coated with Histidine (HIS) tagged human SIRPα.

HIS tagged human SIRPα (ACRO #SIG-H5225) is adsorbed to high-binding microtiter plates at a concentration of 1 μg/ml diluted in PBS overnight at 4° C. The coating solution is removed, the wells are washed and then blocked with 75% casein in PBS containing 0.5% Tween 20 (PBST) for 60 minutes at room temperature while shaking. Blocking solution is removed, the wells are washed and incubated for 60 minutes at room temperature while shaking with anti-SIRP mAbs diluted in PBST at a starting concentration of 30 μg/ml and reducing the concentration by 3-fold serial dilutions. Wells are washed three times with PBST and incubated for 60 minutes at room temperature while shaking with an FC tagged human CD47 (ACRO #CD7-H5256) at a concentration of 250 ng/ml in PBST. Wells are washed three times with PBST and incubated for 60 minutes at room temperature while shaking with an HRP labeled donkey anti-mouse or anti-human secondary antibody (Jackson ImmunoResearch Laboratories) diluted 1:20,000 in PBST. The wells are washed and then incubated with peroxidase substrate and the absorbance at 450 nm determined. The IC₅₀ was calculated using a non-linear fit model (GraphPad Prism).

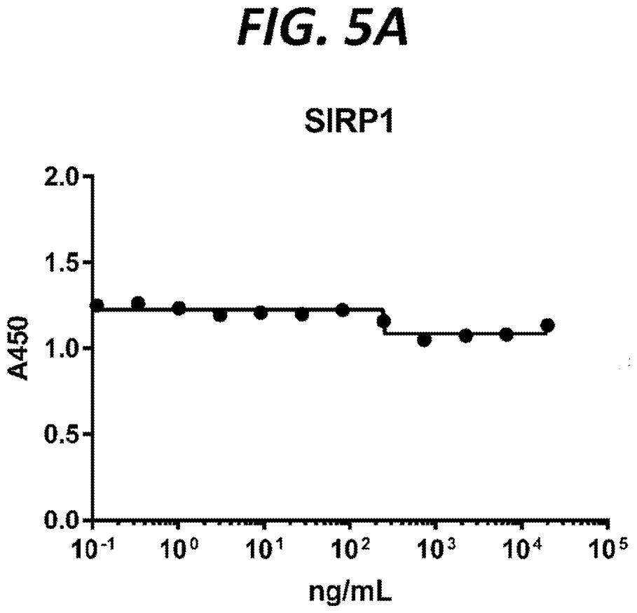

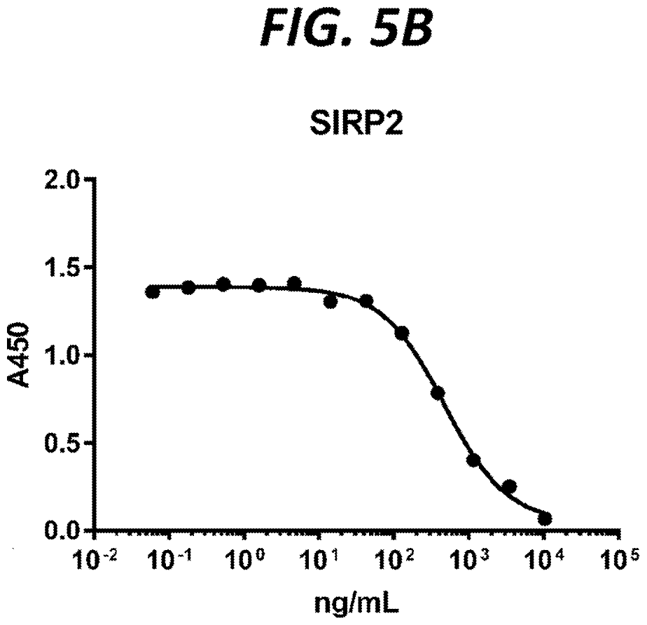

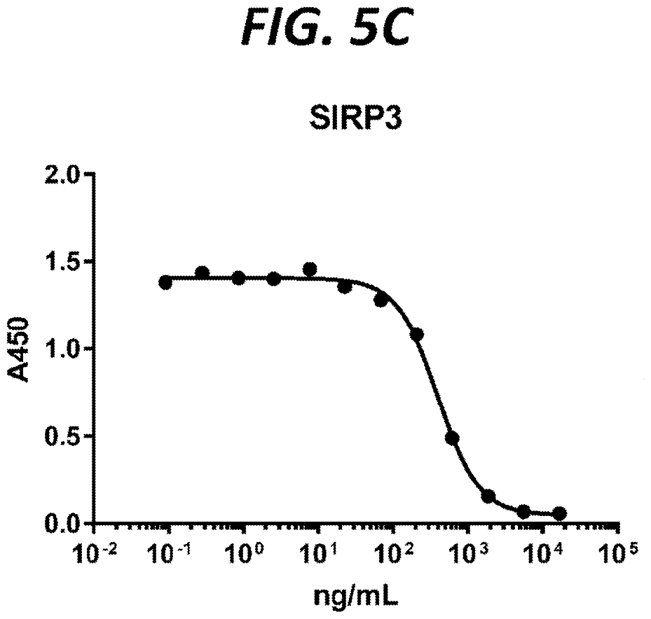

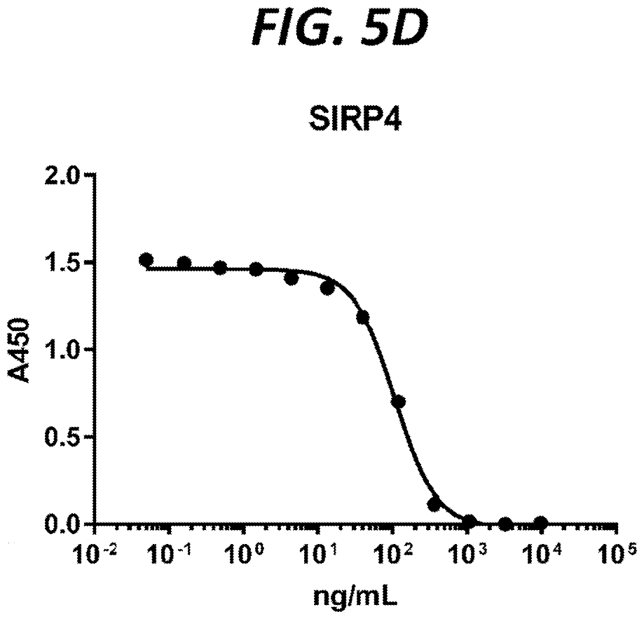

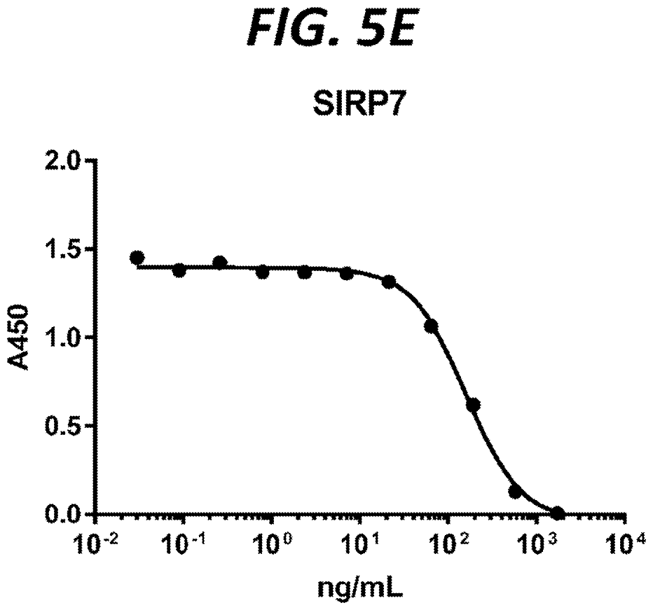

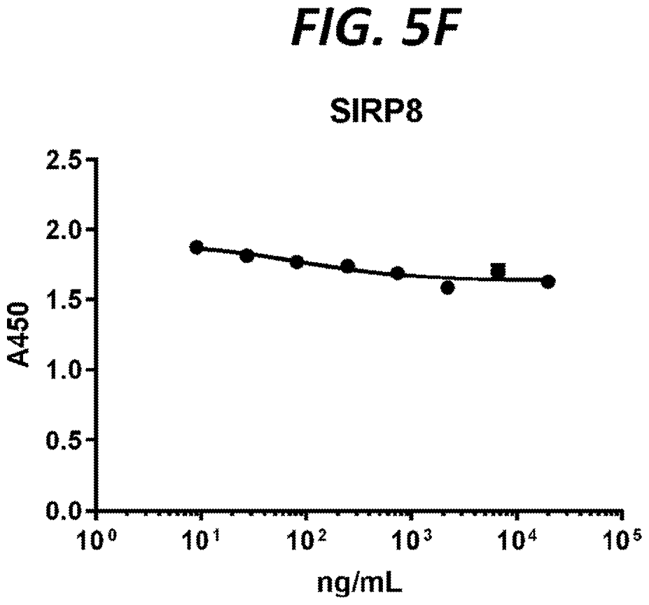

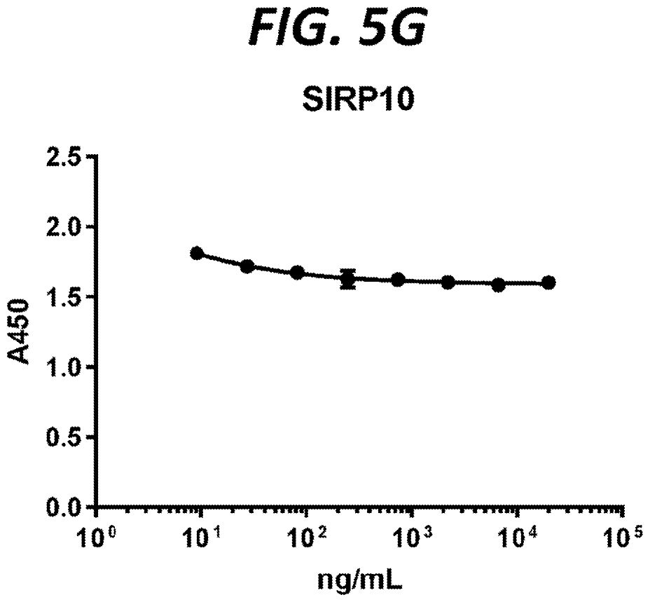

As shown in Table 3, the soluble anti-SIRP mAbs SIRP2, SIRP3, SIRP4, and SIRP7 block the binding of human SIRPα to human CD47 with IC₅₀ values in the nanomolar range. In addition, the soluble anti-SIRP mAbs SIRP1, SIRP5, SIRP6, SIRP8, and SIRP10 were unable to block the binding of human SIRPα to human CD47 at mAb concentrations of up to 30 μg/ml. FIG. 5A-FIG. 5G demonstrates representative inhibition curves derived from antibodies of the present disclosure.

TABLE 3
Blocking of CD47/SIRPα Binding by anti-SIRP Antibodies.

| | SIRPα Blocking |
|---|---|
| | (IC₅₀ nM) |
| SIRP1 | *NB |
| SIRP2 | 3 |
| SIRP3 | 2.7 |
| SIRP4 | 0.71 |
| SIRP5 | *NB |
| SIRP6 | *NB |
| SIRP7 | 1.1 |
| SIRP8 | *NB |
| SIRP10 | *NB |

*NB—no blocking detected at mAb concentration of up to 30 μg/ml

### Example 7

### Anti-SIRP Monoclonal Antibodies Block CD47/SIRPγ Binding

To assess the effect of anti-SIRP mAbs of the present disclosure on binding of CD47 to SIRPγ in vitro the following method was employed using ELISA plates coated with HIS tagged human CD47.

HIS tagged human CD47 (ACRO #CD7-H5227) is adsorbed to high-binding microtiter plates at a concentration of 2 μg/ml diluted in PBS overnight at 4° C. The coating solution is removed, the wells are washed and then blocked with 75% casein in PBS containing 0.5% Tween 20 (PBST) for 60 minutes at room temperature while shaking. Blocking solution is removed, the wells are washed and incubated for 60 minutes at room temperature while shaking with anti-SIRP mAbs diluted in PBST at a starting concentration of 30 μg/ml and reducing the concentration in 3 fold serial dilutions and 0.5 μg/ml of human SIRPγ (ACRO #SIG-H5253). Wells are washed three times with PBST and incubated for 60 minutes at room temperature while shaking with an HRP labeled donkey anti-mouse or anti-human secondary antibody (Jackson ImmunoResearch Laboratories) diluted 1:20,000 in PBST. The wells are washed and then incubated with peroxidase substrate and the absorbance at 450 nm determined. The IC₅₀ was calculated using a non-linear fit model (GraphPad Prism).

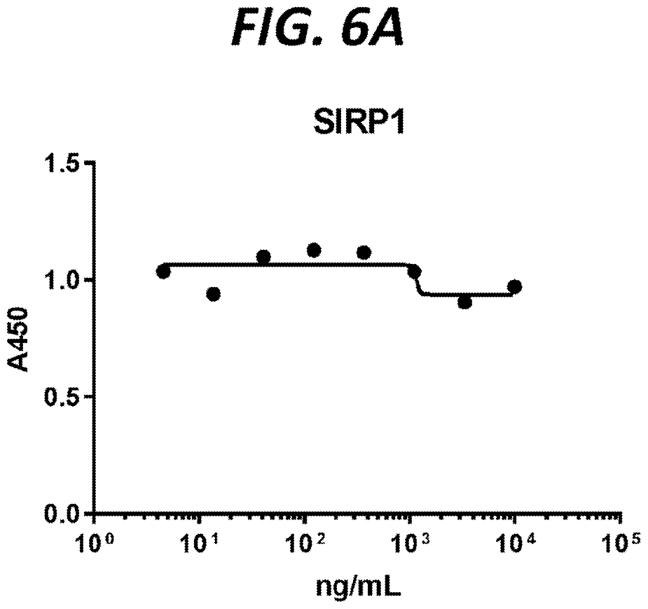

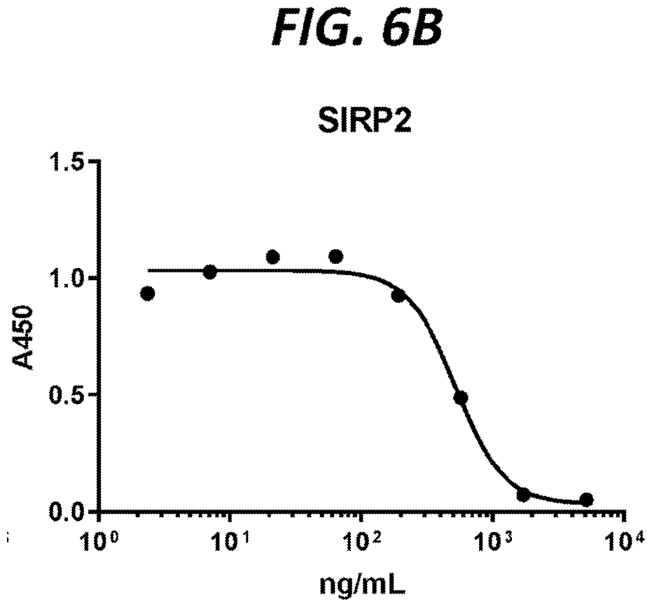

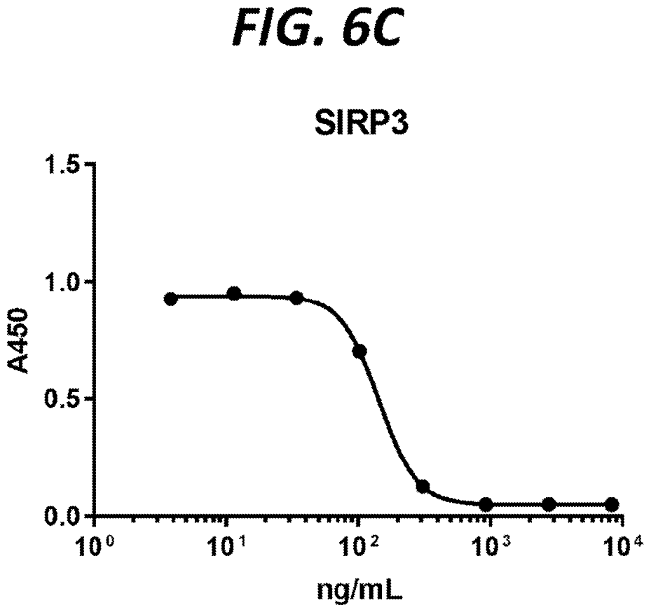

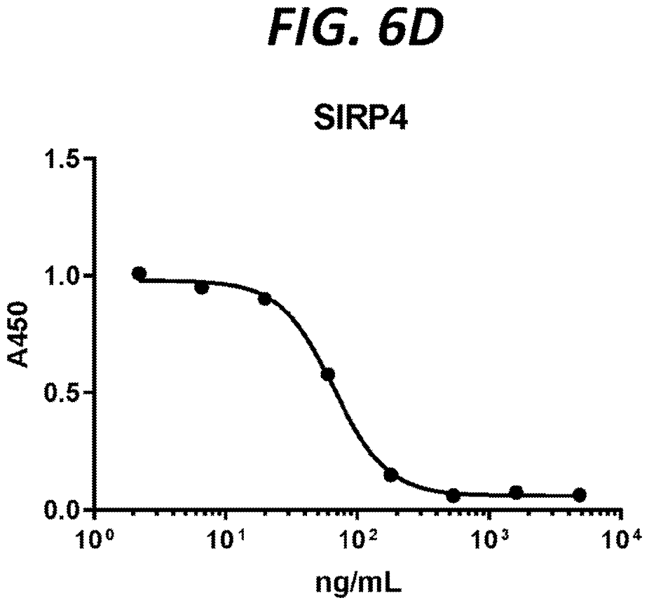

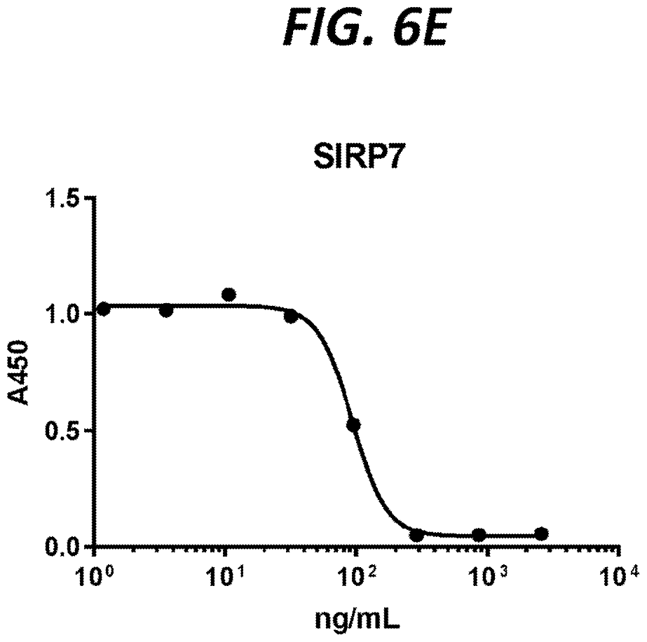

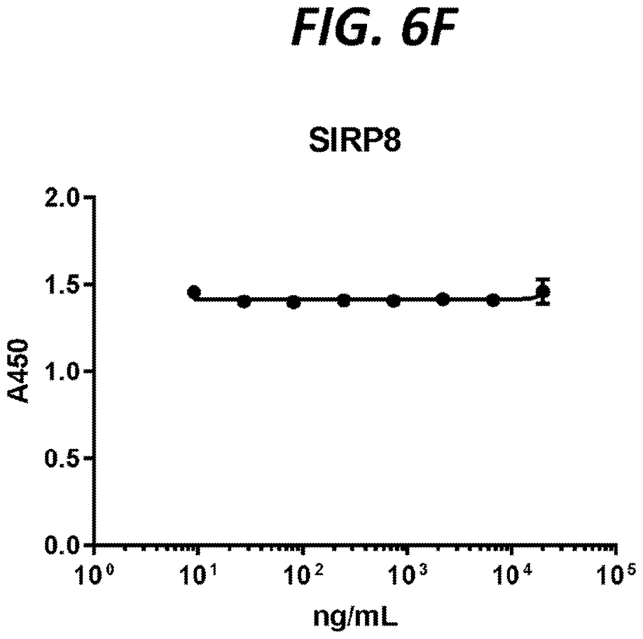

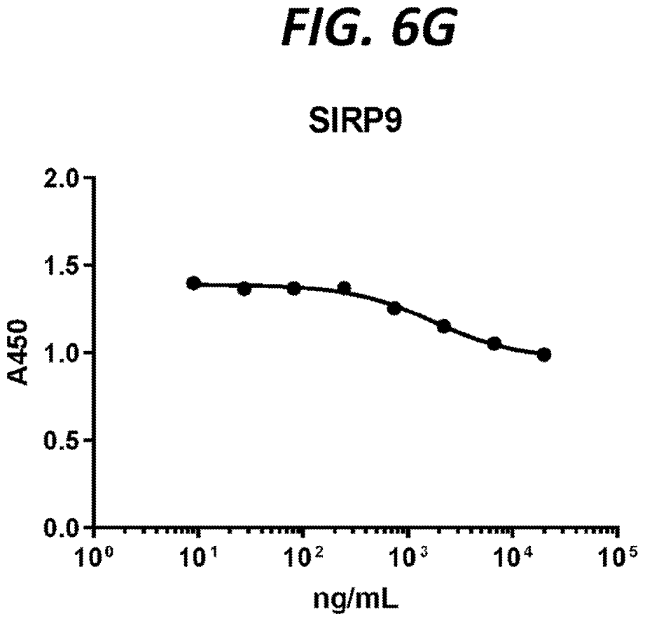

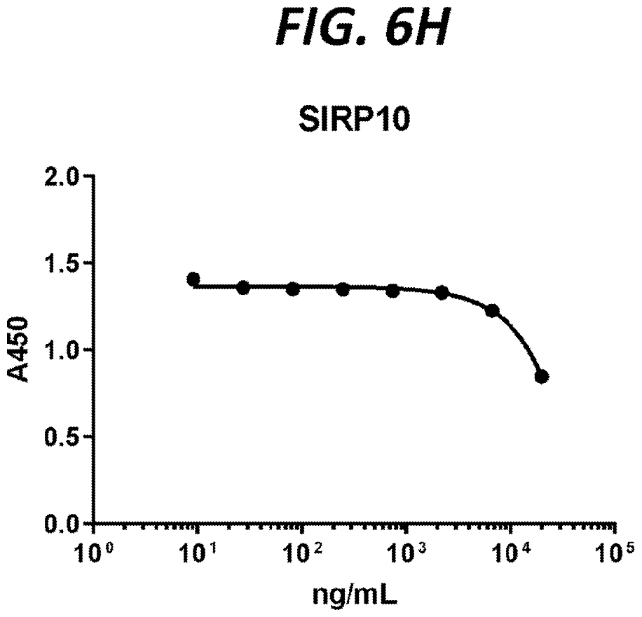

As shown in Table 4, the soluble anti-SIRP mAbs SIRP2, SIRP3, SIRP4, SIRP5, SIRP6, and SIRP7 block the binding of human SIRPγ to human CD47 with IC₅₀ values in the nanomolar range. In addition, the soluble anti-SIRP mAbs SIRP1, SIRP8, SIRP9, and SIRP10 were unable to block the binding of human SIRPγ to human CD47 at mAb concentrations up to 30 μg/ml. FIG. 6A-FIG. 6H demonstrates representative inhibition curves derived from antibodies of the present disclosure.

TABLE 4
Blocking of CD47/SIRPγ Binding by anti-SIRP Antibodies.

| | SIRPγ Blocking |
|---|---|
| | (IC₅₀ nM) |
| SIRP1 | *NB |
| SIRP2 | 3.5 |
| SIRP3 | 0.96 |
| SIRP4 | 0.44 |
| SIRP5 | 0.163 |
| SIRP6 | 0.86 |
| SIRP7 | 0.63 |
| SIRP8 | *NB |
| SIRP9 | *NB |
| SIRP10 | *NB |

*NB—no blocking detected at mAb concentration up to 30 μg/ml

### Example 8

### Anti-SIRP mAbs Induce Phagocytosis

To assess the effect of anti-SIRP mAbs on phagocytosis of tumor cells by macrophages in vitro the following method was employed using flow cytometry.

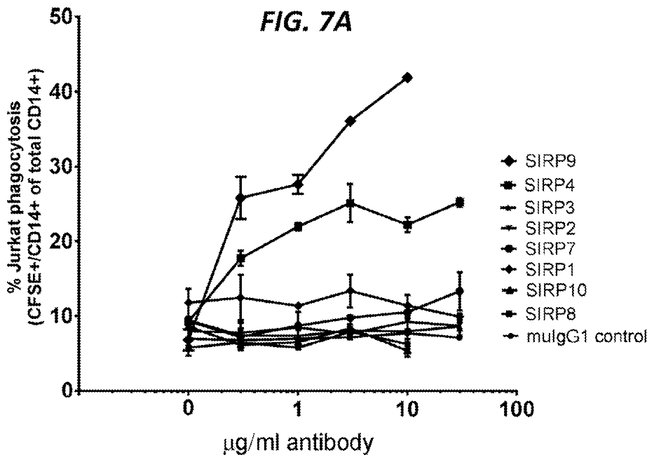

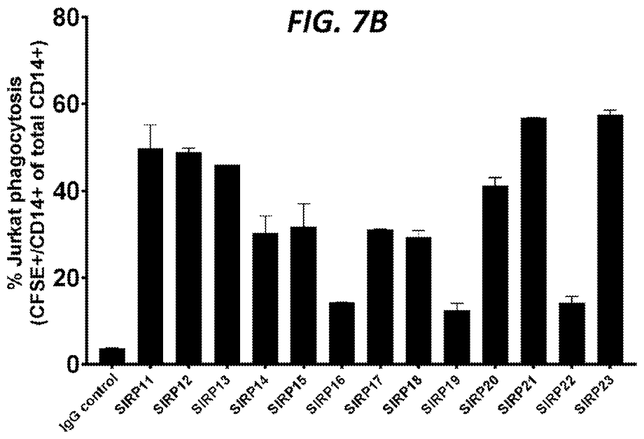

Human monocyte-derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer cells (Jurkat) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP mAbs were added at various concentrations, FIG. 7A, or 10 g/ml of the antibodies, FIG. 7B, immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺, indicating complete phagocytosis.

As shown in FIG. 7A and FIG. 7B, the soluble anti-SIRP mAbs SIRP4, SIRP9, SIRP11, SIRP12, SIRP13, SIRP14, SIRP15, SIRP16, SIRP17, SIRP18, SIRP19, SIRP20, SIRP21, SIRP22 and SIRP23 induced phagocytosis of Jurkat cells by human macrophages as compared to a murine IgG1 control antibody (Biolegend). In contrast, soluble anti-SIRP mAbs SIRP1, SIRP2, SIRP3, SIRP7, SIRP8 and SIRP10 did not induce the phagocytosis of Jurkat cells by human macrophages.

### Example 9

### Anti-SIRP mAbs Induce Phagocytosis when Combined with an Anti-CD47 Antibody

To assess the effect of anti-SIRP mAbs and anti-CD47 mAbs in combination on inducing phagocytosis of tumor cells by macrophages in vitro the following method was employed using flow cytometry.

Human monocyte-derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer cells (Jurkat) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP mAbs alone, an anti-CD47 mAb (known to induce phagocytosis) alone, or anti-SIRP and anti-CD47 mAbs together were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺ indicating complete phagocytosis.

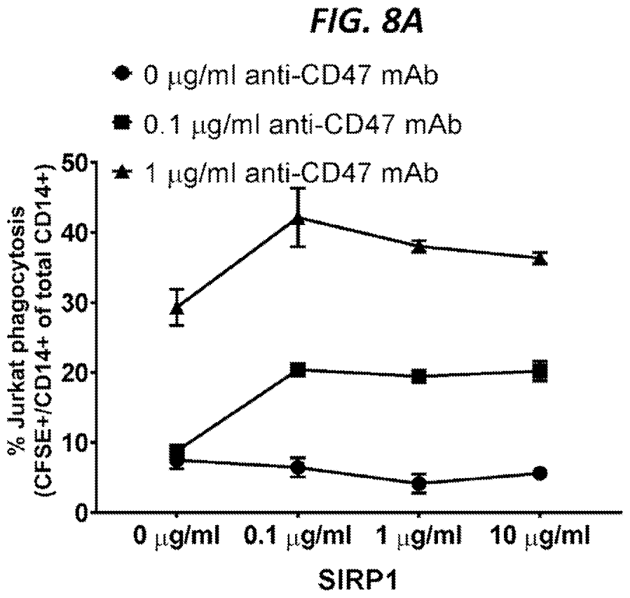

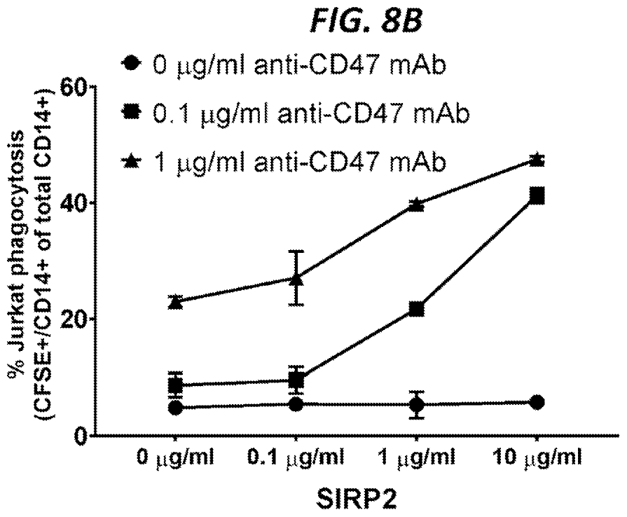

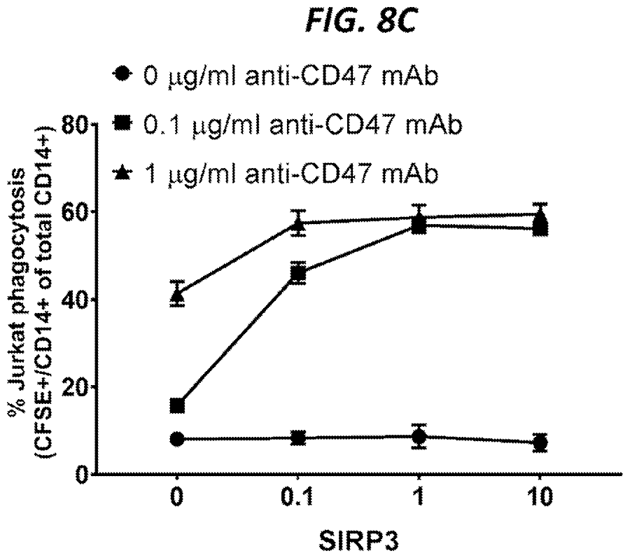

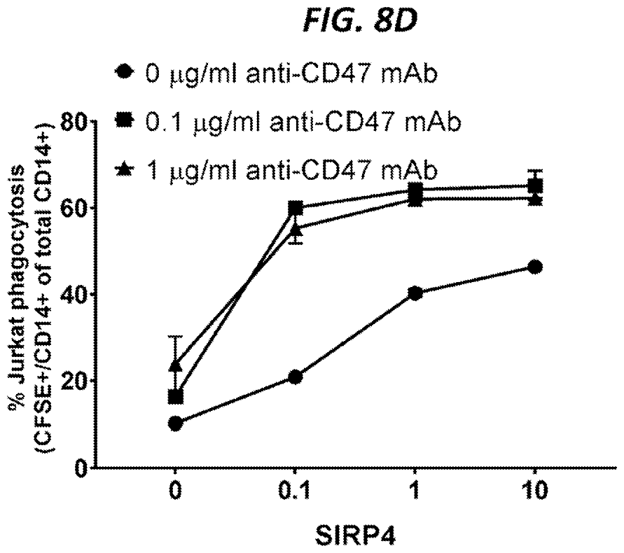

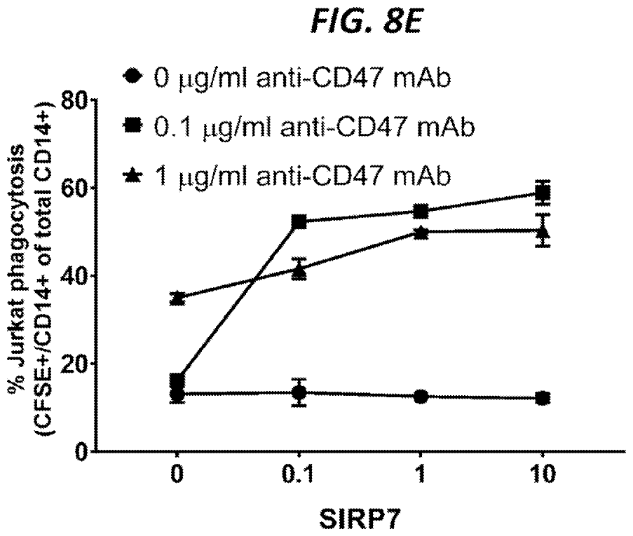

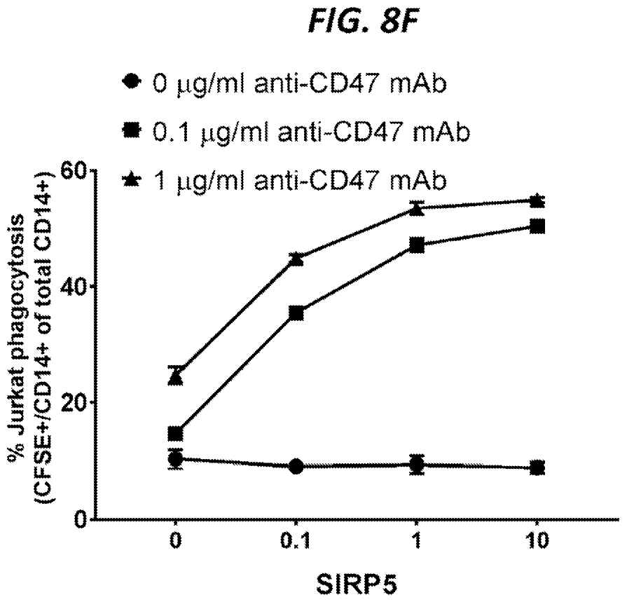

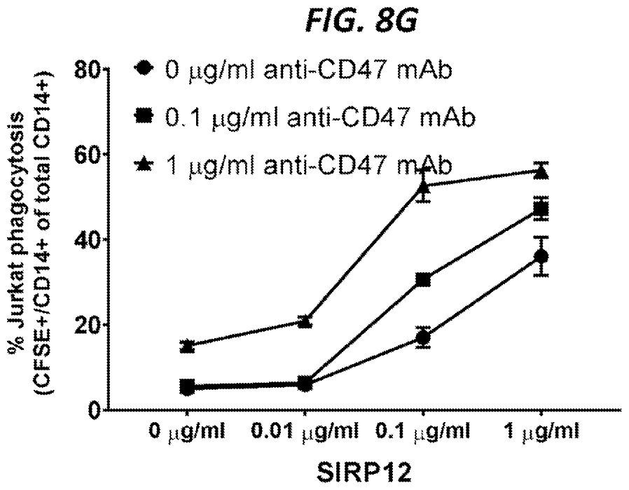

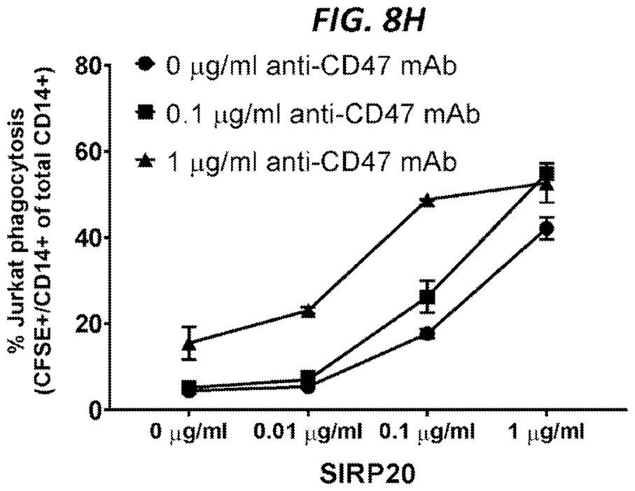

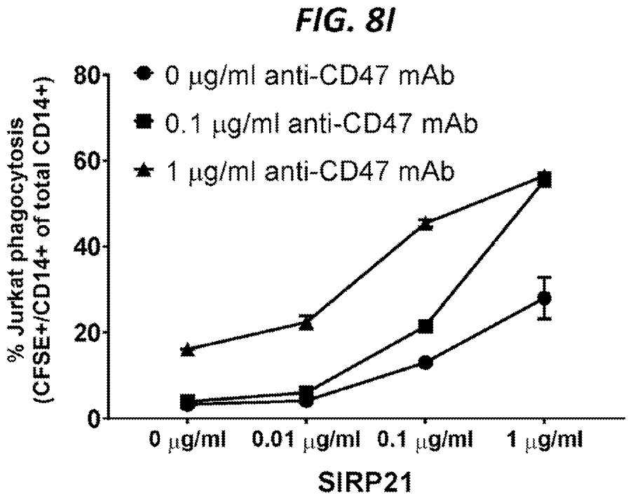

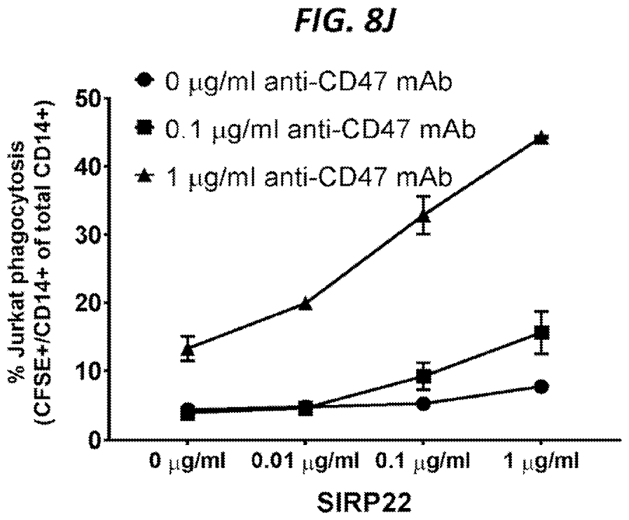

As shown in FIG. 8A-FIG. 8J, all soluble anti-SIRP mAbs SIRP1, SIRP2, SIRP3, SIRP4, SIRP5, SIRP7, SIRP12, SIRP20, SIRP21 and SIRP22 increase phagocytosis of Jurkat cells by human macrophages to a greater degree when combined with anti-CD47 mAbs compared to either agent alone.

### Example 10

### Anti-SIRP mAbs Induce Phagocytosis in Combination with Rituxan

To assess the effect of anti-SIRP mAbs and anti-CD20 mAbs in combination on inducing phagocytosis of tumor cells by macrophages in vitro the following method was employed using flow cytometry.

Human monocyte-derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer cells (RAJI) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP mAbs alone, an anti-CD20 mAb (Rituxan, Roche) alone, or anti-SIRP and anti-CD20 mAbs together were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺ indicating complete phagocytosis.

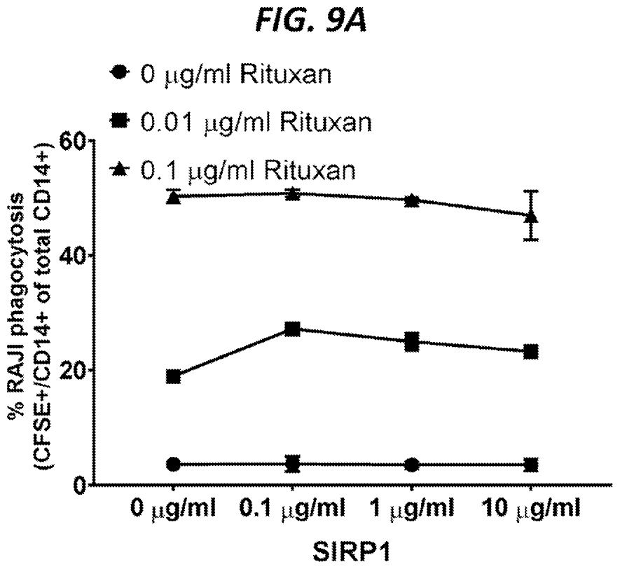

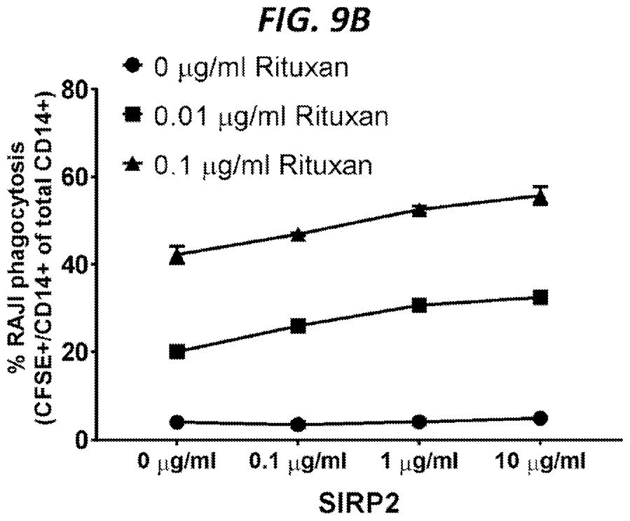

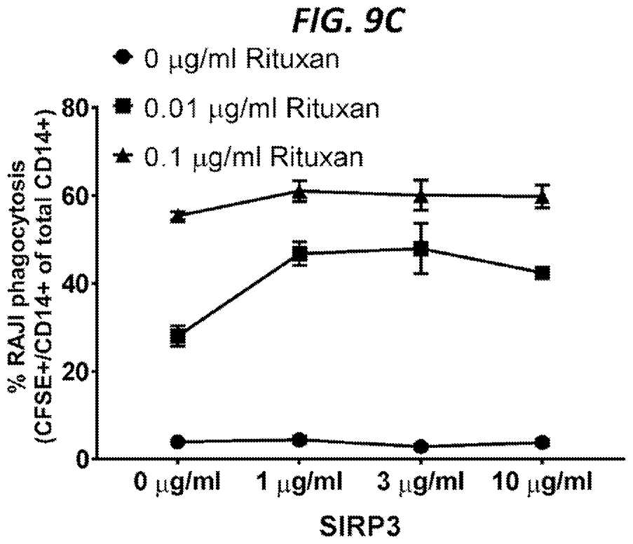

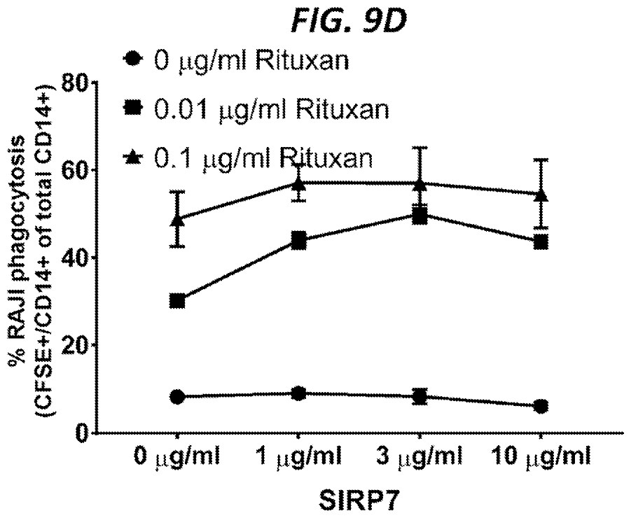

As shown in FIG. 9A-FIG. 9D, all soluble anti-SIRP mAbs SIRP1, SIRP2, SIRP3, and SIRP7 increased phagocytosis of RAJI cells by human macrophages to a greater degree when combined with anti-CD20 mAbs compared to either agent alone.

### Example 11

### Anti-SIRP mAbs Induce Phagocytosis in Combination with Erbitux and Avelumab

To assess the effect of anti-SIRP mAbs and anti-EGFR mAbs or anti-PD-L1 mAbs in combination on inducing phagocytosis of tumor cells by macrophages in vitro the following method was employed using flow cytometry.

Human monocyte-derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, the targeted human cancer cells (FaDu or ES-2) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP mAbs alone, an anti-EGFR mAb (Erbitux, Bristol-Myers Squibb) alone, an anti-PD-L1 mAb (Avelumab, Pfizer), or anti-SIRP and anti-EGFR mAbs together were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺ indicating complete phagocytosis.

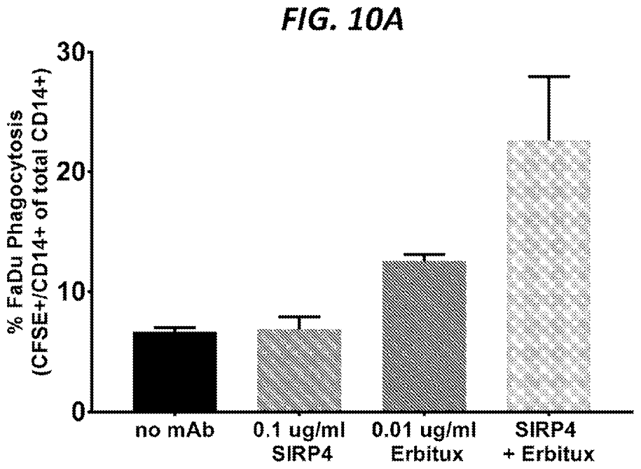

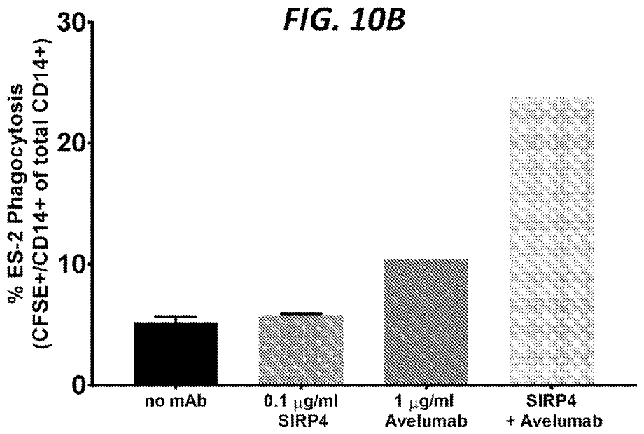

As shown in FIG. 10A, soluble anti-SIRP mAb SIRP4 increased phagocytosis of FaDu cells by human macrophages to a greater degree when combined with anti-EGFR mAbs compared to either agent alone. As shown in FIG. 10B, soluble anti-SIRP mAb SIRP4 increased phagocytosis of ES-2 cells by human macrophages to a greater degree when combined with anti-PD-L1 mAbs compared to either agent alone.

### Example 12

### Anti-SIRP mAbs Bind to Human Macrophages and Dendritic Cells

To assess the binding of anti-SIRP mAbs to cells expressing SIRPα such as human macrophages and dendritic cells the following method was employed using flow cytometry.

Human CD14⁺ monocytes, isolated from peripheral blood mononuclear cells (Astarte Biologics) were differentiated in vitro for seven days into macrophages or dendritic cells. For macrophage differentiation, monocytes were incubated in AIM-V media (Life Technologies) supplemented with 50 ng/ml M-CSF (Biolegend) for seven days. For dendritic cell differentiation, monocytes were incubated in AIM-V media (Life Technologies) in the presence of 10% human AB serum (Valley Biomedical), 200 ng/ml GM-CSF (Biolegend) and 50 ng/ml IL-4 (Biolegend). The cells were incubated for 1 h at 37° C., 5% CO₂ with serial dilutions of SIRP mAbs in binding buffer containing 1 mM EDTA (Sigma Aldrich) and 1% FBS (Biowest) in PBS (Corning). The cells were then washed and stained for 45 min under the same conditions with donkey anti-mouse IgG fluorescein isothiocyanate (FITC)-linked secondary antibody (Jackson ImmunoResearch Laboratories). The cells were subsequently stained with anti-CD14 or anti-CD11c conjugated to Alexa Fluor 647 fluorophore (Life Technologies and Biolegend, respectively) for 30 min on ice, washed and analyzed by flow cytometry (Attune, Life Technologies). Binding was assessed as the median FITC fluorescence intensity of CD14⁺ or CD11c⁺ cells, subtracted from cells stained with the secondary antibody only.

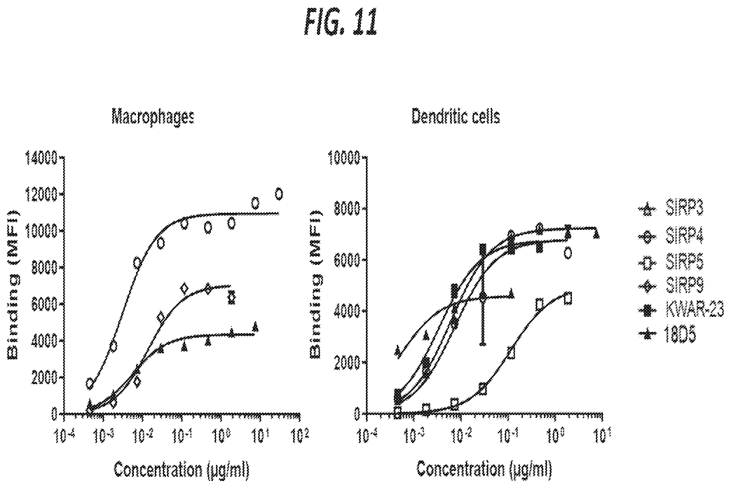

As shown in Table 5, the soluble anti-SIRP mAbs SIRP3, SIRP4, SIRP5 and SIRP9, as well as OSE-18D5 and KWAR-23, bound to cell-expressed SIRPα on dendritic cells and/or macrophages with apparent affinities in the picomolar range. FIG. 11 demonstrates representative binding curves derived from the antibodies of the present disclosure.

TABLE 5
Binding of anti-SIRP mAbs to Human Cells Expressing SIRPα.

| | Human macrophage | Human dendritic |
|---|---|---|
| | binding K_{d} (pM) | cell binding K_{d} (pM) |
| SIRP3 | ND* | 3.47 |
| SIRP4 | 20.7 | 50 |
| SIRP5 | ND* | 770 |
| SIRP9 | 93.7 | ND* |
| 18D5 | 37.3 | 41.2 |
| KWAR-23 | ND* | 23.4 |

*Not Determined

### Example 13

### Anti-SIRP mAbs Exhibit Variable Binding to Human CD3⁺ T Cells

To assess the binding of anti-SIRP mAbs on human CD3 T cells the following method was employed using flow cytometry.

Human CD3 T cells, isolated from peripheral blood mononuclear cells (Astarte Biologics) were incubated in 96-well V-bottom plates at 2.5×10⁵ cells/well for 1 h at 37° C., 5% CO₂ with serial dilutions of SIRP mAbs in binding buffer containing 1 mM EDTA (Sigma Aldrich), 1% FBS (Biowest) in PBS (Corning). The cells were then washed and stained for 45 min under the same conditions with donkey anti-mouse IgG fluorescein isothiocyanate (FITC)-linked secondary antibody (Jackson ImmunoResearch Laboratories). The cells were subsequently stained with anti-CD3 conjugated to 3-carboxy-6,8-difluoro-7-hydroxycoumarin (Pacific Blue) fluorophore (BioLegend) for 30 min on ice, washed and analyzed by flow cytometry (Attune, Life Technologies). Binding was assessed as the median FITC fluorescence intensity of CD3⁺ cells, subtracted from CD3⁺ cells stained with the secondary antibody only. All SIRP antibodies were generated in-house except for LSB2.20 (BioLegend). For activated T cells, prior to the binding assay CD3 T cells were activated for 72 h in a 96-well flat-bottom plate coated with 10 μg/ml anti-CD3 (clone UCHT1; BioLegend), at 1×10⁵ cells/well in the presence of 0.5 μg/ml anti-CD28 (clone CD28.2; BioLegend).

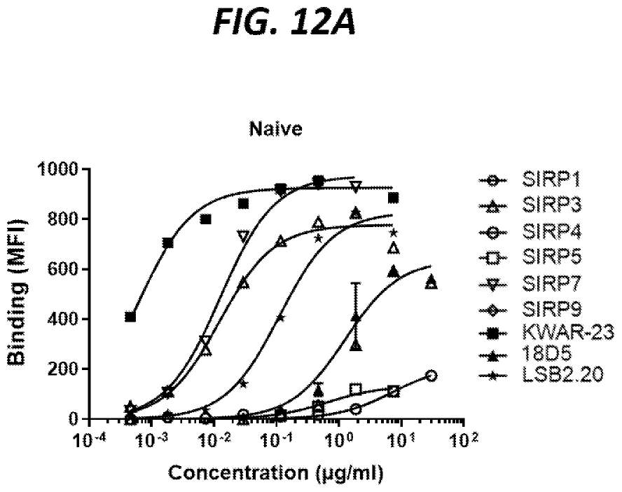

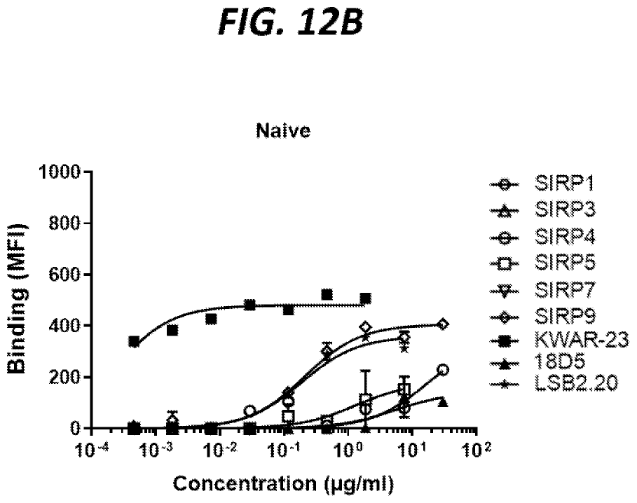

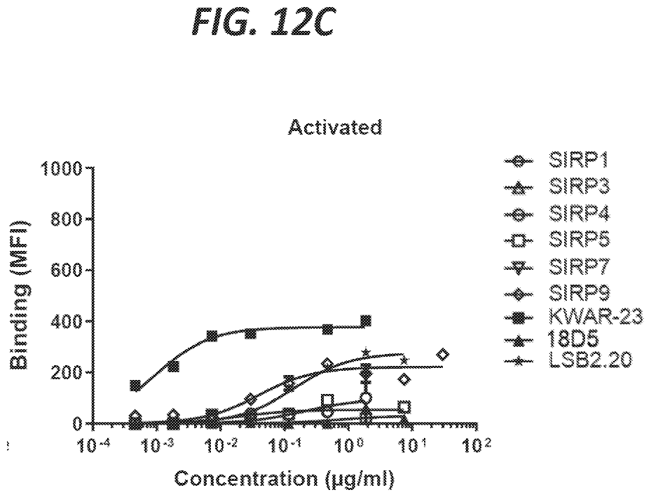

As shown in Table 6, the soluble SIRP3, SIRP7, SIRP9, KWAR-23, and the SIRPγ-specific antibody LSB2.20 bind T cells with affinities in the picomolar range. The affinities of anti-SIRP mAbs SIRP4, SIRP5 and OSE-18D5 are much lower and are in the nanomolar range. FIG. 12A, FIG. 12B, and FIG. 12C demonstrate representative binding curves derived from antibodies of the present disclosure.

TABLE 6
Binding of anti-SIRP Antibodies to
Human T Cells Expressing SIRPγ.

| | Human T cell | Human T cell | Human T cell |
|---|---|---|---|
| | binding K_{d} (pM) | binding K_{d} (pM) | binding K_{d} (pM) |
| | Naive | Naive | Activated |
| SIRP3 | 80.7 | ND | ND |
| SIRP4 | NC* | NC* | NC* |
| SIRP5 | NC* | NC* | NC* |
| SIRP7 | 83.9 | ND | ND |
| SIRP9 | ND | 1410 | 263 |
| 18D5 | 8410 | NC* | NC* |
| KWAR-23 | 4.04 | 1.59 | 6.22 |
| LSB2.20 | 750 | 1260 | 950 |

*NC Not calculated; mean fluorescence intensities were comparable to the mIgG1 background level
** Not determined.

### Example 14

### Anti-SIRP mAbs Do Not Block Soluble CD47/Cellular SIRPγ Binding

To assess the effect of anti-SIRP antibodies of the present disclosure on blocking the binding of soluble CD47 to cells expressing SIRPγ, the following method was employed using soluble human IgG1 Fc tagged human CD47.

Human T-ALL cells (Jurkat) were incubated at 2.5×10⁵ cells/well for 1 h at 37° C., 5% CO₂ with 10 μg/ml of anti-SIRP mAbs in binding buffer containing 1 mM EDTA (Sigma Aldrich), 1% FBS (Biowest) in PBS (Corning). Following this, soluble human IgG1 Fc tagged human CD47 (ACRO #CD7-H5256) was added for a final concentration of 50 μg/ml and the cells incubated as previously for another 1 h. The cells were then washed extensively and stained for 45 min under the same conditions with donkey anti-human antibody conjugated to Alexa Fluor 647 (Jackson ImmunoResearch). The samples were analyzed by flow cytometry (Attune, Life Technologies). For analysis, background human IgG1 Fc staining in the absence of soluble Fc tagged CD47 was subtracted from median Alexa Fluor 647 fluorescence intensity. Blocking was assessed as the reduction in background-corrected median fluorescence intensity of Alexa Fluor 647 in the presence of SIRP mAbs compared to murine IgG1 (Biolegend, MOPC-21) control.

As shown in Table 7, the soluble anti-SIRP mAbs SIRP4, SIRP9, and OSE 18D5 do not block the binding of cell expressed SIRPγ to soluble human CD47. KWAR-23 does block the binding of Jurkat cell expressed SIRPγ to soluble human CD47.

TABLE 7
Blocking of CD47/SIRPγ Binding by anti-SIRP Antibodies.

| | Blocking of soluble CD47 |
|---|---|
| | binding to SIRPγ on Jurkat |
| SIRP4 | Non-blocking |
| SIRP9 | Non-blocking |
| OSE 18D5 | Non-blocking |
| KWAR-23 | Blocking |

### Example 15

### Anti-SIRP mAbs Block Soluble CD47/Cellular SIRPα Binding

To assess the effect of anti-SIRP antibodies of the present disclosure on binding of soluble CD47 to cells expressing SIRPα, the following method was employed using human macrophages and soluble human IgG1 Fc tagged human CD47.

Human CD14⁺ monocytes, isolated from peripheral blood mononuclear cells (Astarte Biologics) were differentiated in vitro for seven days in AIM-V media (Life Technologies) supplemented with 50 ng/ml M-CSF (Biolegend). Macrophage Fc receptors were then blocked with human Fc receptor blocking solution (Biolegend) for 20 min at room temperature. The cells were then washed and incubated for 1 h at 37° C., 5% CO₂ with 10 μg/ml of anti-SIRP mAbs in binding buffer containing 1 mM EDTA (Sigma Aldrich), 1% FBS (Biowest) in PBS (Corning). Following this, soluble human IgG1 Fc tagged human CD47 (ACRO #CD7-H5256) was added for a final concentration of 20 μg/ml and the cells incubated as previously for another 1 h. The cells were then washed extensively and stained for 45 min under the same conditions with donkey anti-human antibody conjugated to Alexa Fluor 647 (Jackson ImmunoResearch). The samples were analyzed by flow cytometry (Attune, Life Technologies). For analysis, background human IgG1 Fc staining in the absence of soluble Fc tagged CD47 was subtracted from median Alexa Fluor 647 fluorescence intensity. Blocking was assessed as the reduction in background-corrected median fluorescence intensity of Alexa Fluor 647 in the presence of SIRP mAbs compared to murine IgG1 (Biolegend, MOPC-21) control. Four different monocyte donors were used in these assays with a minimum of three donors per antibody tested.

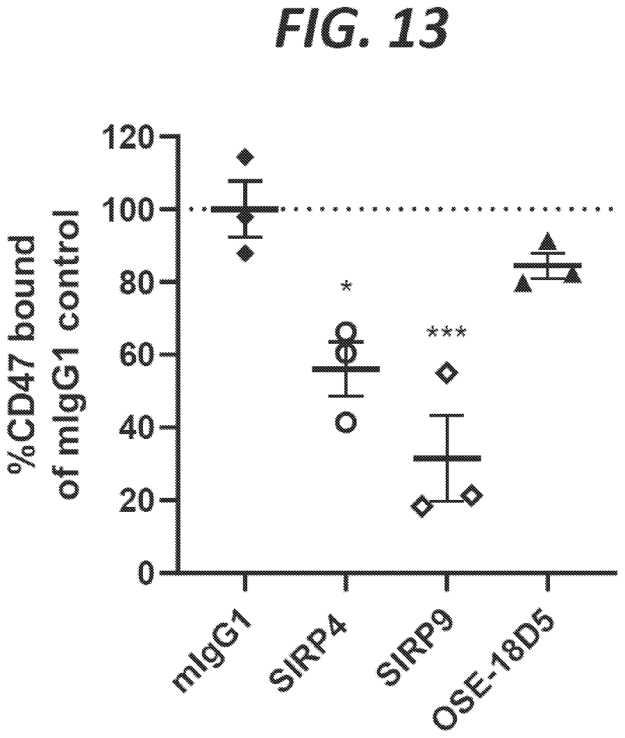

As shown in FIG. 13, the soluble anti-SIRP mAbs SIRP4 and SIRP9 block the binding of cell expressed SIRPα on macrophages to soluble human CD47. The OSE 18D5 mAb does not block the binding of cell expressed SIRPα to soluble human CD47.

### Example 16

### Anti-SIRP mAbs do not Inhibit T Cell Proliferation

To assess the effect of anti-SIRP mAbs on allogeneic dendritic cell-induced T cell proliferation in vitro the following method was employed using flow cytometry.

Human monocyte-derived dendritic cells were generated by incubating CD14⁺ monocytes (Astarte Biologics) in AIM-V medium (Life Technologies) supplemented with 10% human AB serum (Valley Biomedical), 200 ng/ml GM-CSF (Biolegend) and 50 ng/ml IL-4 (Biolegend) for six days, with addition of fresh, cytokine replete medium on Day 2. For the allogeneic dendritic cell and T cell co-culture assay, immature dendritic cells were re-plated onto a 96-well plate at a concentration of 1×10⁵ cells per well. CellTrace™ Violet (Life Technologies) fluorescent cell proliferation dye-labelled allogeneic healthy donor derived CD3⁺ T cells from four different donors (Astarte Biologics) were added to the culture at a 1:5 DC: T cell ratio. Anti-SIRP mAbs were added immediately at the saturating concentration of 10 μg/ml immediately and the cells incubated at 37° C., 5% CO₂ for 6-7 days in a total volume of 200 l. Cells were then detached by scraping the wells with pipette tips and washed in fluorescence-activated cell sorting buffer (1% FBS, Biowest, in PBS). Cells were then incubated with PerCP-Cy5.5 fluorescent dye labelled CD3 antibody (Biolegend) for 30 minutes on ice, washed once, and analyzed by flow cytometry (Attune, Life Technologies). T cell proliferation was measured by the dilution of the CellTrace™ Violet dye within the CD3⁺ cell population.

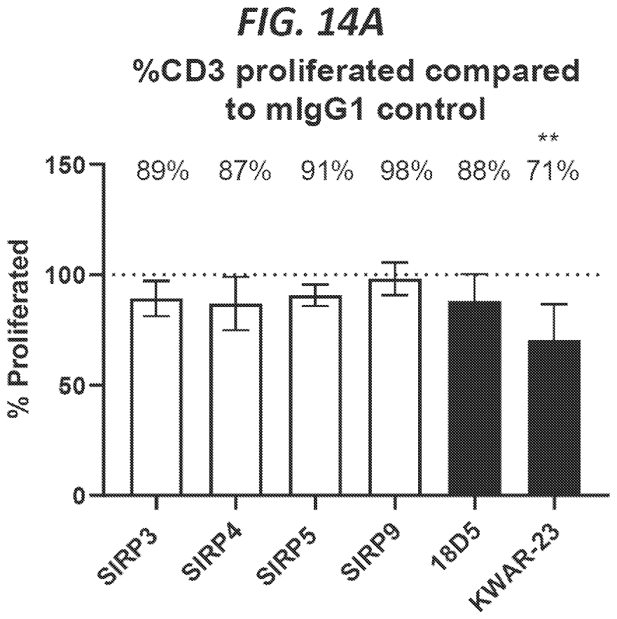

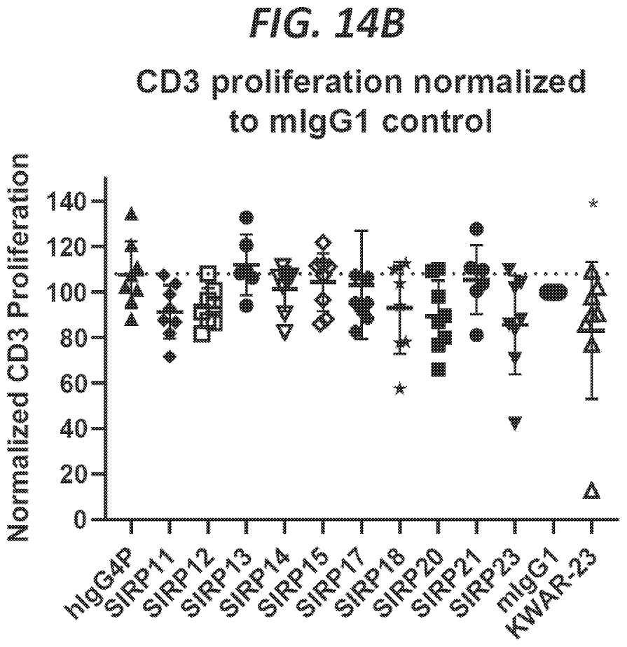

As shown in FIG. 14A and FIG. 14B, the anti-SIRP mAbs SIRP3, SIRP4, SIRP5, SIRP9, SIRP11, SIRP12, SIRP13, SIRP14, SIRP15, SIRP17, SIRP18, SIRP20, SIRP21, SIRP23 and OSE-18D5 had no significant effect on T cell proliferation compared to control antibody (Biolegend). In contrast, KWAR-23, which blocks both SIRPα and SIRPγ binding to CD47, inhibited T cell proliferation.

### Example 17

### Anti-SIRP mAbs do not Inhibit Antigen-Specific T Cell Recall Response

To assess the effect of anti-SIRP mAbs on antigen recall response in T cells in vitro the following method was employed using flow cytometry.

Human peripheral blood mononuclear cells from a cytomegalovirus seropositive donor (Astarte Biologics) were labelled with CellTrace™ Violet (Life Technologies) fluorescent cell proliferation dye and seeded at 200,000 cells/well in a 96-well plate. The cells were then incubated with different concentrations of cytomegalovirus antigen (Astarte Biologics) in AIM-V medium (Life Technologies) supplemented with 10% human AB serum (Valley Biomedical), which induces an antigen dependent stimulation of T cell proliferation. Anti-SIRP mAbs as well as an anti-CD47 mAb, clone B6H12, (Biolegend) were added immediately at the saturating concentration of 10 μg/ml immediately and the cells incubated at 37° C., 5% C02 for five days. T cell proliferation was measured by the dilution of the CellTrace™ Violet dye within the CD4+ cell population.

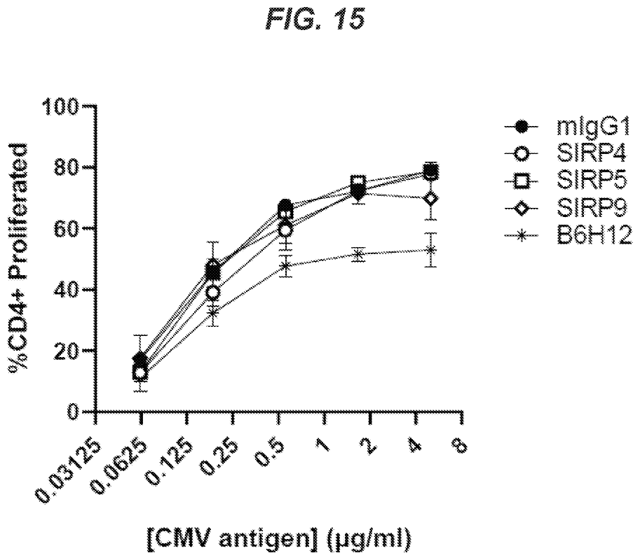

As shown in FIG. 15, the soluble anti-SIRP mAbs SIRP4, SIRP5 and SIRP9 did not inhibit the ability of T cells to elicit a CMV antigen recall response. In contrast, the anti-CD47 antibody clone B6H12, which is known to inhibit T cell responses, reduced T cell proliferation compared to murine IgG1 control antibody (Biolegend).

### Example 18

SIRPα Antibody Sequences

| | LCDR1 | LCDR2 | LCDR3 | HCDR1 | HCDR2 | HCDR3 |
|---|---|---|---|---|---|---|
| | (SEQ ID | (SEQ ID | (SEQ ID | (SEQ ID | (SEQ ID | (SEQ ID |
| Antibody | NO:) | NO:) | NO:) | NO:) | NO:) | NO:) |
| SIRP1 | 1 | 2 | 3 | 33 | 34 | 35 |
| SIRP2 | 4 | 5 | 6 | 36 | 37 | 38 |
| SIRP3 | 7 | 8 | 9 | 39 | 40 | 41 |
| SIRP4 | 10 | 11 | 12 | 42 | 43 | 44 |
| SIRP5 | 13 | 14 | 15 | 45 | 46 | 47 |
| SIRP6 | 16 | 17 | 18 | 48 | 49 | 50 |
| SIRP7 | 19 | 20 | 21 | 51 | 52 | 53 |
| SIRP8 | 22 | 23 | 24 | 54 | 55 | 56 |
| SIRP9 | 25 | 26 | 27 | 57 | 58 | 59 |
| SIRP10 | 28 | 29 | 30 | 60 | 61 | 62 |
| SIRP11 | 10 | 31 | 12 | 42 | 43 | 44 |
| SIRP12 | 10 | 31 | 12 | 42 | 43 | 44 |
| SIRP13 | 10 | 31 | 32 | 42 | 43 | 44 |
| SIRP14 | 10 | 31 | 12 | 42 | 43 | 44 |
| SIRP15 | 10 | 31 | 12 | 42 | 43 | 44 |
| SIRP16 | 10 | 31 | 32 | 42 | 43 | 44 |
| SIRP17 | 10 | 31 | 12 | 42 | 43 | 44 |
| SIRP18 | 10 | 31 | 12 | 42 | 43 | 44 |
| SIRP19 | 10 | 31 | 32 | 42 | 43 | 44 |
| SIRP20 | 10 | 31 | 12 | 42 | 43 | 44 |
| SIRP21 | 10 | 31 | 12 | 42 | 43 | 44 |
| SIRP22 | 10 | 31 | 32 | 42 | 43 | 44 |
| SIRP23 | 10 | 11 | 12 | 42 | 43 | 44 |
| SIRP24 | 25 | 26 | 27 | 57 | 58 | 63 |
| SIRP25 | 25 | 26 | 27 | 57 | 58 | 63 |
| SIRP26 | 25 | 26 | 27 | 57 | 58 | 63 |
| SIRP27 | 25 | 26 | 27 | 57 | 58 | 63 |
| SIRP28 | 25 | 26 | 27 | 57 | 58 | 63 |
| SIRP29 | 25 | 26 | 27 | 57 | 58 | 63 |
| SIRP30 | 25 | 26 | 27 | 57 | 58 | 59 |
| SIRP31 | 25 | 26 | 27 | 57 | 58 | 59 |
| SIRP32 | 25 | 26 | 27 | 57 | 58 | 59 |
| SIRP33 | 25 | 26 | 27 | 57 | 58 | 63 |
| | V_{L} | V_{H} | LC | HC | | |
| | (SEQ ID | (SEQ ID | (SEQ ID | (SEQ ID | | |
| | NO:) | NO:) | NO:) | NO:) | | |
| SIRP1 | 64 | 81 | 98 | 109 | | |
| SIRP2 | 65 | 82 | 99 | 110 | | |
| SIRP3 | 66 | 83 | 100 | 111 | | |
| SIRP4 | 67 | 84 | | | | |
| SIRP5 | 68 | 85 | | | | |
| SIRP6 | 69 | 86 | | | | |
| SIRP7 | 70 | 87 | | | | |
| SIRP8 | 71 | 88 | | | | |
| SIRP9 | 72 | 89 | | | | |
| SIRP10 | 73 | 90 | | | | |
| | LCDR1 | LCDR2 | LCDR3 | HCDR1 | HCDR2 | HCDR3 |
| | (SEQ ID | (SEQ ID | (SEQ ID | (SEQ ID | (SEQ ID | (SEQ ID |
| Antibody | NO:) | NO:) | NO:) | NO:) | NO:) | NO:) |
| SIRP11 | 74 | 91 | 101 | 112 | | |
| SIRP12 | 75 | 91 | 102 | 112 | | |
| SIRP13 | 76 | 91 | 103 | 112 | | |
| SIRP14 | 74 | 92 | 101 | 113 | | |
| SIRP15 | 75 | 92 | 102 | 113 | | |
| SIRP16 | 76 | 92 | 103 | 113 | | |
| SIRP17 | 74 | 93 | 101 | 114 | | |
| SIRP18 | 75 | 93 | 102 | 114 | | |
| SIRP19 | 76 | 93 | 103 | 114 | | |
| SIRP20 | 74 | 94 | 101 | 115 | | |
| SIRP21 | 75 | 94 | 102 | 115 | | |
| SIRP22 | 76 | 94 | 103 | 115 | | |
| SIRP23 | 77 | 84 | 104 | 116 | | |
| SIRP24 | 78 | 95 | 105 | 117 | | |
| SIRP25 | 79 | 95 | 106 | 117 | | |
| SIRP26 | 80 | 95 | 107 | 117 | | |
| SIRP27 | 78 | 96 | 105 | 118 | | |
| SIRP28 | 79 | 96 | 106 | 118 | | |
| SIRP29 | 80 | 96 | 107 | 118 | | |
| SIRP30 | 78 | 97 | 105 | 119 | | |
| SIRP31 | 79 | 97 | 106 | 119 | | |
| SIRP32 | 80 | 97 | 107 | 119 | | |
| SIRP33 | 72 | 89 | 108 | 120 | | |

### Example 19

### Anti-SIRP Monoclonal Antibody Effect on SIRPα Architecture

To assess the effect of anti-SIRP antibodies of the present disclosure on the architecture of SIRPα, its organization in dimers, multimers, or clusters, the following fluorescence resonance energy transfer (FRET) assayed by flow cytometry (FCET) method is employed using human macrophages and non-competing SIRPα antibodies labeled with phycoerythrin (PE) or allophycocyanin (APC).

Human CD14⁺ monocytes, isolated from peripheral blood mononuclear cells, (Astarte Biologics) were differentiated in vitro for seven days in AIM-V medium (Life Technologies) supplemented with 50 ng/ml M-CSF (BioLegend). For FRET assay PE and APC labelled antibodies against SIRPα were used according to a FCET methodology previously described (Batard et al., 2002). FRET between an antibody-PE conjugated donor and an antibody-APC conjugated acceptor indicate that the two molecules are in close proximity. Human macrophages were detached from plates using Accutase, washed and incubated in AIM-V medium for 2 h in V-bottom 96-well plates. The cells were then incubated with 10 μg/ml SIPR4, SIRP9, or mIgG1 control for 2 h at 37° C., 5% CO₂. Subsequently, the cells were washed, and equimolar concentration of SIRP antibody clone SE5A5-PE and SE5A5-APC (both from BioLegend) were combined (for 10 μg/ml final) and added to the cell suspension. Cells were also labelled with SE5A5-PE or SE5A5-APC in the presence of equimolar quantities of unlabeled SE5A5 (BioLegend) for single fluorophore staining. The cells were stained for 30 min on ice, washed and analyzed on a flow cytometer (Attune, Life Technologies). FRET efficiency was calculated using three wavelength correction method (Batard et al., 2002) with the assumption of 1:1 protein-to-dye ratio for both SE5A5-APC and SE5A5-PE (using information provided by BioLegend).

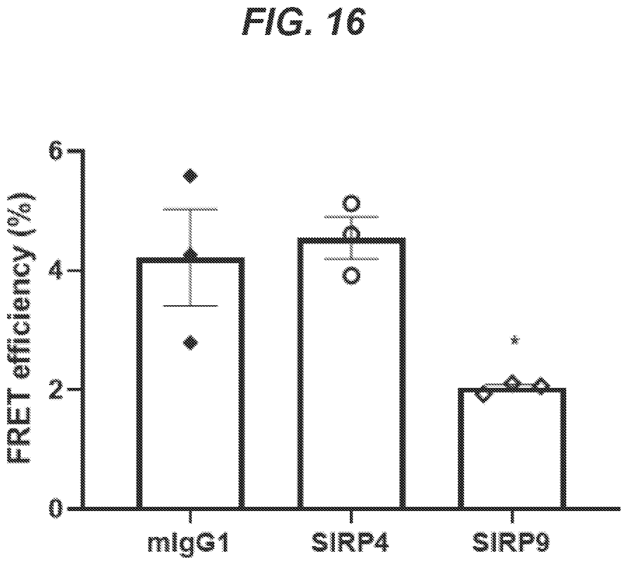

As shown in FIG. 16, the anti-SIRP mAb SIRP9 reduces the FRET efficiency between SE5A5-PE donor and SE5A5-APC acceptor. The anti-SIRP mAb SIRP4 does not reduce the FRET efficiency between SE5A5-PE donor and SE5A5-APC acceptor.

### Example 20

### Anti-SIRP Monoclonal Antibody Internalization by Macrophages

To assess the effect of anti-SIRP antibodies of the present disclosure on internalization of SIRPα-antibody complexes, the following method was employed using human macrophages and pHrodo green-labelled anti-SIRP antibodies. The pHrodo green dye only fluoresces once in the acidic, internalized compartment.

Human CD14⁺ monocytes, isolated from peripheral blood mononuclear cells, (Astarte Biologics) were differentiated in vitro for seven days in AIM-V medium (Life Technologies) supplemented with 50 ng/ml M-CSF (BioLegend). SIRP antibodies were labelled using pHrodo Green Microscale Protein Labeling Kit (Invitrogen), per manufacturer's instructions. Labeling efficiency was comparable between all the antibodies. The antibodies were diluted into macrophage growth medium and warmed to 37° C. At indicated times, media was removed from macrophages and replaced with medium containing 10 μg/ml labeled antibody and the incubation continued at 37° C. for a maximum of 1 h. After incubation, the medium was removed, and the cells detached from the plate using Accutase (STEMCELL Technologies). Cells were pelleted at 400 g, washed ice-cold PBS (Corning), and analyzed by flow cytometry (Attune, Life Technologies). Internalization was quantified as the percentage of pHrodo green⁺ live single cells.

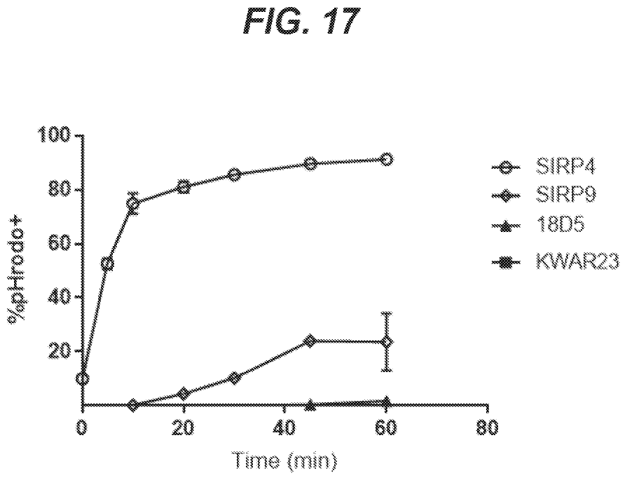

As shown in FIG. 17, the soluble anti-SIRP mAb SIRP4 induces internalization of SIRPα-antibody complexes. To a lesser extent, the soluble anti-SIRP9 mAb induces internalization of SIRP alpha-antibody complexes.

### Example 21

### Anti-SIRP Monoclonal Antibodies Reduce Cell Surface SIRP Alpha Levels

To assess the effect of anti-SIRP antibodies of the present disclosure on the expression level of SIRP alpha on the cell surface, the following flow cytometry-based method was used utilizing human macrophages.

Human CD14⁺ monocytes, isolated from peripheral blood mononuclear cells, (Astarte Biologics) were differentiated in vitro for seven days in AIM-V medium (Life Technologies) supplemented with 50 ng/ml M-CSF (Biolegend). Human macrophages were detached from plates using Accutase (STEMCELL Technologies), washed and incubated in AIM-V medium for 2 h in V-bottom 96-well plates. The cells were then incubated with 10 μg/ml mIgG1 control, SIPR4, SIRP9, 18D5 or KWAR23 for 2 h at 4° C. or for 2 h, 4 h, 6 h or 24 h at 37° C., 5% CO₂. Subsequently, the cells were washed in flow cytometry (FC) buffer (2% FBS in PBS), and the cell surface SIRP alpha level measured using non-competing fluorescently labelled anti-SIRP alpha antibodies. For SIRP4, 18D5 and KWAR23, Alexa Fluor 647-labelled anti-SIRP antibody SIRP9 was used. SIRP9-AF647 was generated using Alexa Fluor 647 Antibody Labeling kit from Molecular Probes by Life Technologies. For SIRP4, phycoerythrin labelled anti-SIRP antibody SE5A5 was used (BioLegend). For fluorescent antibody labeling, the cells were stained for 30 min on ice, washed and analyzed on a flow cytometer (Attune, Life Technologies). Fluorescence levels were normalized to median fluorescence intensity of mIgG1 control treated macrophages, stained with the respective fluorescent SIRP antibodies on ice.

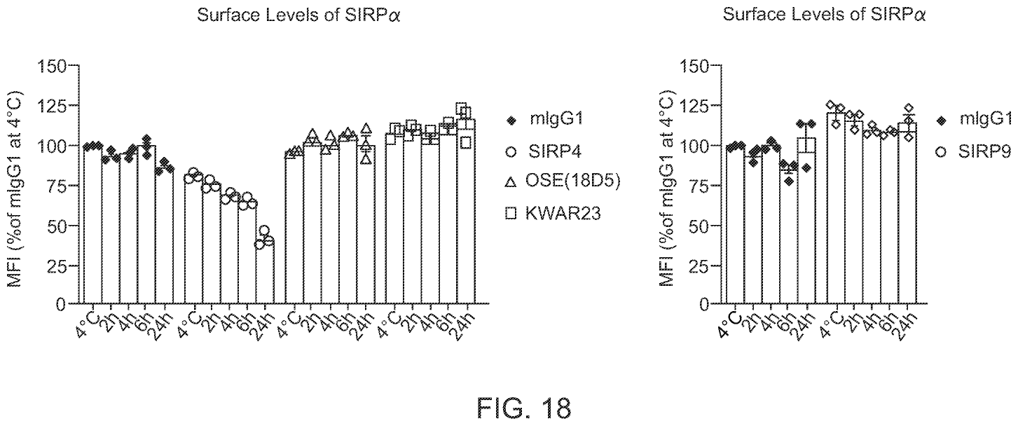

As shown in FIG. 18, anti-SIRP mAb SIRP4 shows a reduction in the surface level of SIRPα. Anti SIRP mAb SIRP9, 18D5 or KWAR23 do not show a reduction in the surface level of SIRP alpha.

### Example 22

### Anti-SIRP Monoclonal Antibodies Increase Phagocytosis of Tumor Cells by Macrophages Irrespective of SIRPα Allelic Variant

To assess the effect of anti-SIRP alpha antibodies on phagocytosis of tumor cells by macrophages bearing different SIRP alpha allelic variants, the following in vitro method is employed using flow cytometry.

Human monocyte derived macrophages (MDMs/MQs) from different SIRPα allelic groups (V1/V1, V1/V2, and V2/V2) were differentiated from CD14⁺ monocytes. CD14⁺ monocytes were either purchased from Astarte Biologics or enriched using the Pan Monocyte Isolation Kit (Miltenyi Biotec) from peripheral blood mononuclear cells (PBMCs, AllCells and Hemacare). Monocytes were seeded onto 96-well flat bottom plates at 5×10⁴ cells/well and differentiated into MDMs in vitro for seven days in AIM-V medium (Life Technologies) supplemented with 10% FBS (Biowest) and 50 ng/ml M-CSF (BioLegend).

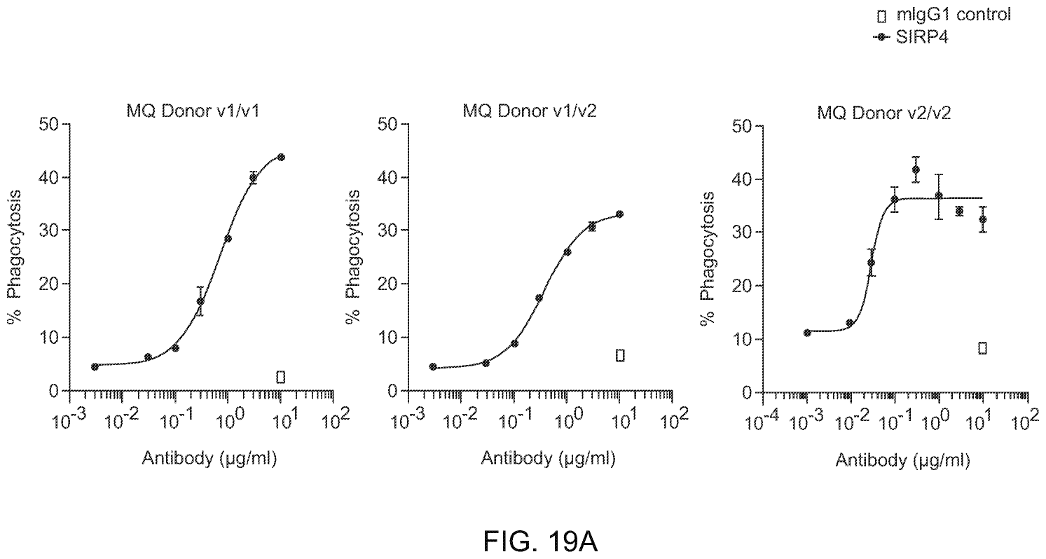

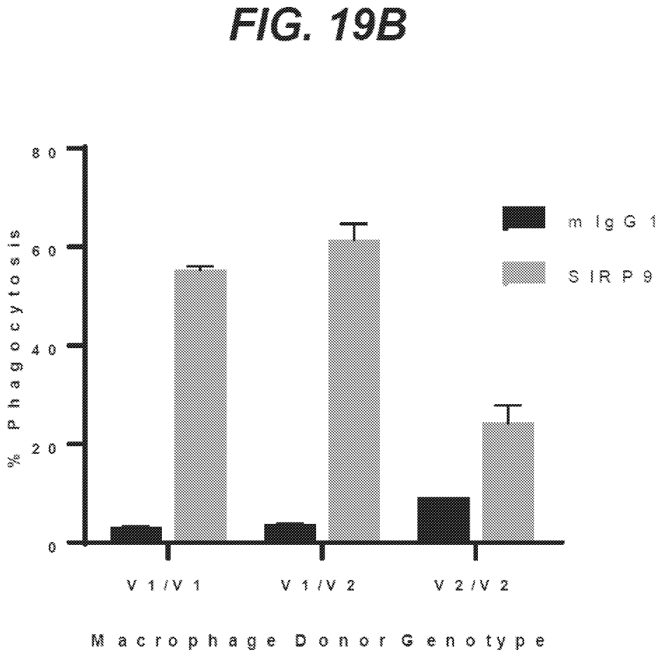

Prior to setting up in vitro phagocytosis assays, human MDMs were cultured in AIM-V medium without supplements for 2 h. Human cancer cells were labeled with 1 μM 5(6)-Carboxyfluorescein diacetate N-succinimidyl ester (CFSE; Sigma Aldrich) and added to the macrophage cultures in 96-well plates at a concentration of 8×10⁴ cells/well in AIM-V medium without supplements. Anti-SIRP or mIgG1 control antibodies were added at various concentrations (FIG. 19A) or at a concentration of 10 μg/ml (FIG. 19B) immediately upon mixture of target and effector cells and allowed to incubate at 37° C. for 4 h. After 4 hours, all non-phagocytosed cells were removed, and the remaining cells washed three times with PBS. Cells were then incubated in Accutase (Innovative Cell Technologies) to detach macrophages, collected into 96-well V-bottom plates, and incubated in 100 ng of allophycocyanin (APC) labeled CD14 antibodies (BD biosciences) for 30 minutes, washed once, and analyzed by flow cytometry (Attune, Life Technologies). Phagocytosis was determined as the percentage of CFSE⁺ cells within the CD14⁺ cell population.

As shown in FIGS. 19A-19B, anti-SIRP antibodies SIRP4 and SIRP9 induce phagocytosis of tumors independent of SIRP alpha allelic variant.

### Example 23

### Anti-SIRP Monoclonal Antibodies do not Compete with Each Other for SIRPα Binding

To assess the competition between anti-SIRP antibodies of the present disclosure in binding to human SIRPα, the following ELISA method is employed using His-tagged human SIRPα protein and biotinylated SIRPα antibodies.

Antibodies against SIRP alpha were biotinylated using EZ-Link™ Sulfo-NHS-LC-Biotinylation Kit (Thermo Scientific), according to manufacturer's instructions and their binding to human SIRPα confirmed using solid phase ELISA. For competitive ELISA, plate adsorbed human SIRPα (His-tagged, ACRObiosystems) was incubated for 1 h at room temperature with serially diluted unbiotinylated SIRP antibodies in the presence of 1.25 μg/ml of biotinylated SIRP antibodies in a pairwise manner. For detection, 1:5000 HRP streptavidin (BioLegend) and peroxidase substrate 3,3′,5,5′-tetramentylbenzidine (TMB, Thermo Scientific) were used, and the reaction stopped by addition of 1N H₂SO₄. The absorbance at 450 nm was determined using a Synergy H1 plate reader (BioTek).

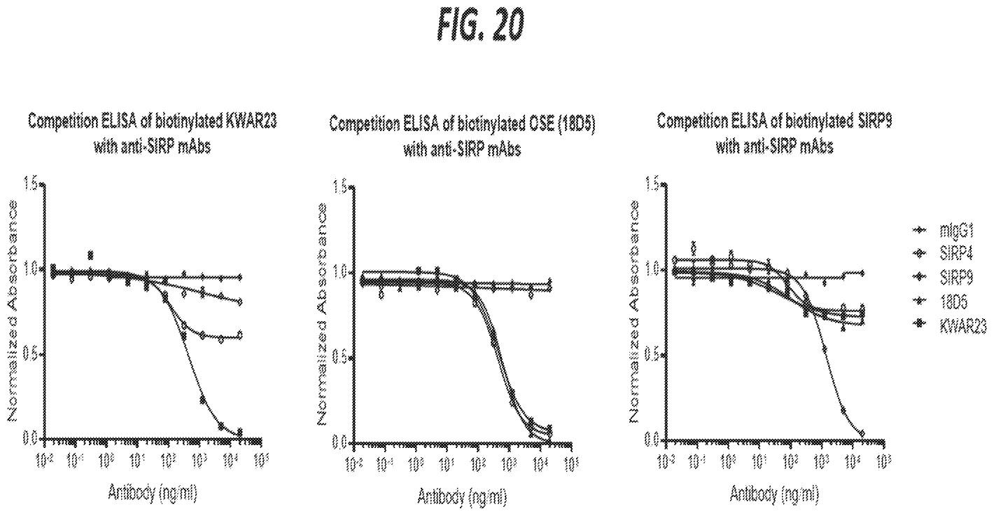

As shown in FIG. 20, anti-SIRP mAb SIRP9 does not compete with SIRP4, OSE (18D5) and KWAR23 for SIRPα binding. Anti-human SIRP mAb SIRP4 competes for SIRPα binding with OSE (18D5) but not SIRP9 or KWAR-23.

### Example 24

### Anti-SIRP Monoclonal Antibodies Exhibit Various Binding Patterns to Human Cancer Cell Lines

To assess the binding of anti-SIRP mAbs to human cancer cell lines used in phagocytosis assays and the expression of SIRPα/β, SIRPγ, and CD47 by these cell lines, the following method is employed using flow cytometry.

Human cancer cell lines (Jurkat T-ALL, RAJI B cell lymphoma, DLD-1 colorectal adenocarcinoma, RL95-2 endometrial carcinoma, and ES-2 ovarian carcinoma) were purchased from American Type Culture Collection (ATCC) and cultured as recommended by the vendor. To assess the levels of SIRPα, SIRPγ and CD47 on these cell lines, non-adherent cell lines were collected in their growth medium and adherent cell lines were detached from their culture plates using Accutase (Stemcell Technologies). The cells were then washed in PBS, placed into 96-well V-bottom plates at 1×10⁵ cells/well and incubated with commercial phycoerythrin (PE) labeled antibodies that recognize human SIRPα/β (clone SE5A5), SIRPγ (clone LSB2.20) or CD47 (clone B6H12), all purchased from BioLegend, for 30 minutes on ice in flow cytometry buffer (1% FBS in PBS). The cells were then washed and analyzed by flow cytometry (Attune, Life Technologies). For measuring binding of SIRP4 and SIRP9 mAbs to cancer cell lines, cells were placed in 96-well V-bottom plates as above for 1 h at 37° C., 5% CO₂ with 10 μg/ml of SIRP mAbs or murine IgG1 control (BioLegend) in binding buffer containing 1 mM EDTA (Sigma Aldrich), 1% FBS (Biowest) in PBS (Corning). The cells were then washed and stained for 45 minutes under the same conditions with donkey anti-mouse IgG fluorescein isothiocyanate (FITC)-linked secondary antibody (Jackson ImmunoResearch Laboratories), then washed and analyzed by flow cytometry (Attune, Life Technologies).

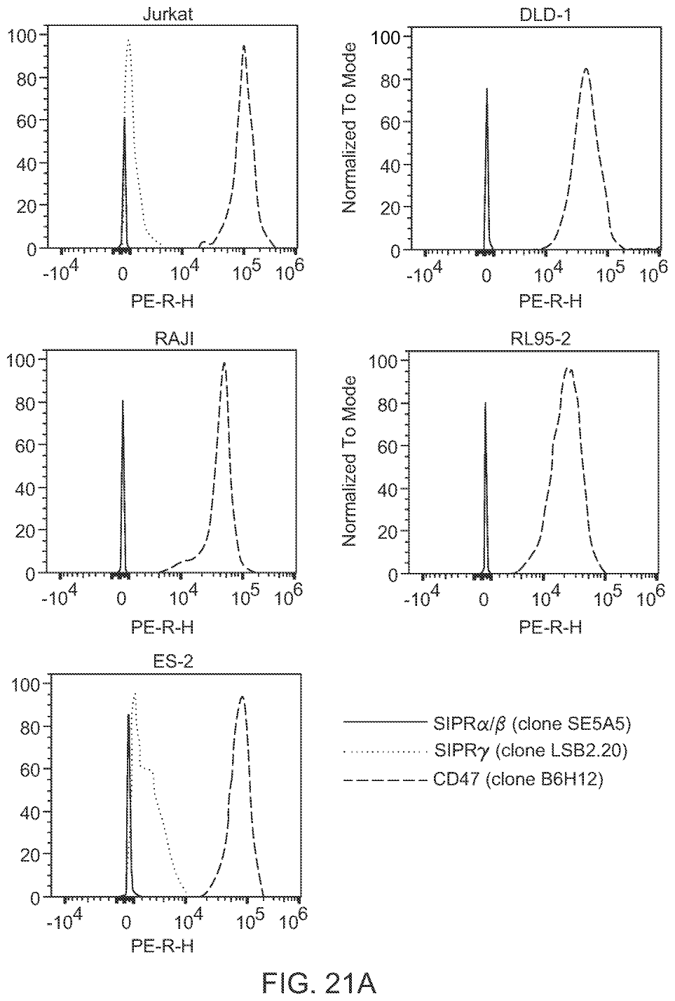

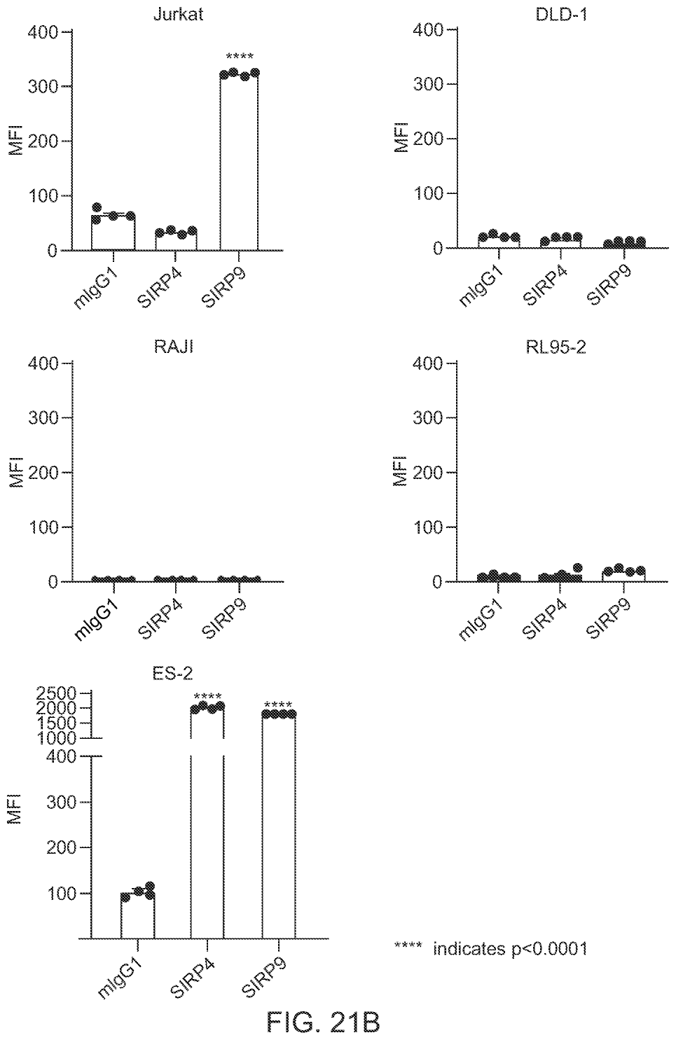

As shown in FIG. 21A, using SIRP mAbs with known specificities, the Jurkat cells express SIRPγ and ES-2 cells express SIRPα/β, whereas the rest of the cell lines do not express either SIRPα/β or SIRPγ. All tested cell lines express CD47. As shown in FIG. 21B, SIRP9 binds to Jurkat (SIRPγ) and ES-2 (SIRPα/β) cell lines and SIRP4 binds only ES-2 (SIRPα/β) cell line, but not to Jurkat (SIRPγ).

### Example 25

### Anti-SIRP mAb-Induced Phagocytosis Depends on FcγR Engagement

To assess the effect of blocking human Fc receptors on anti-SIRP mAb induced phagocytosis of tumor cells by macrophages in vitro the following method was employed using flow cytometry.

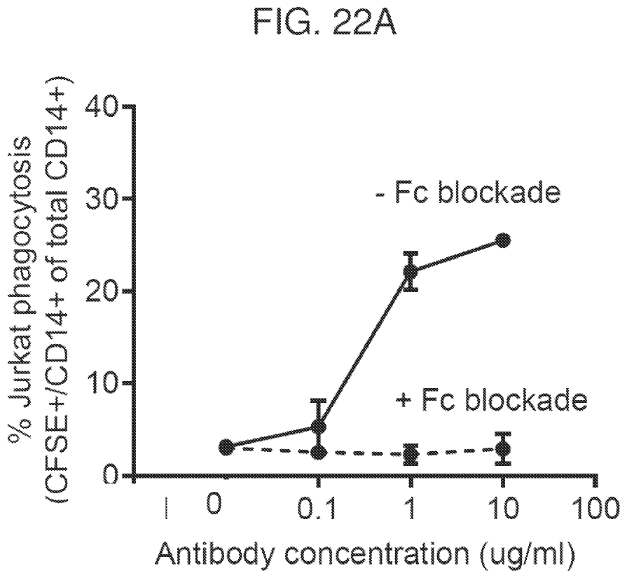

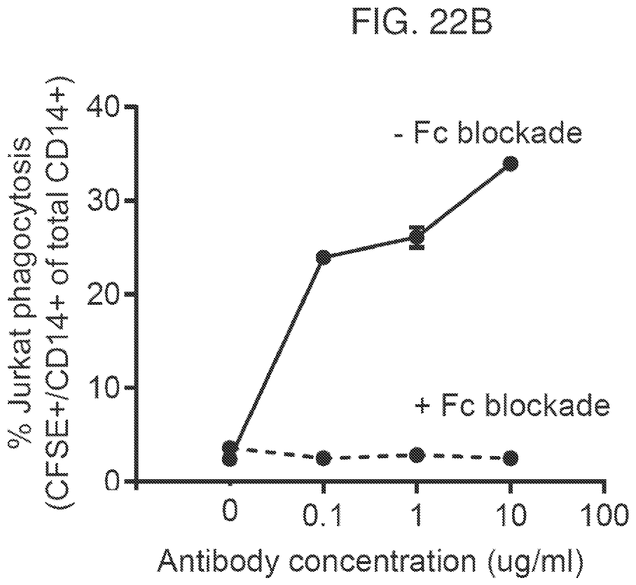

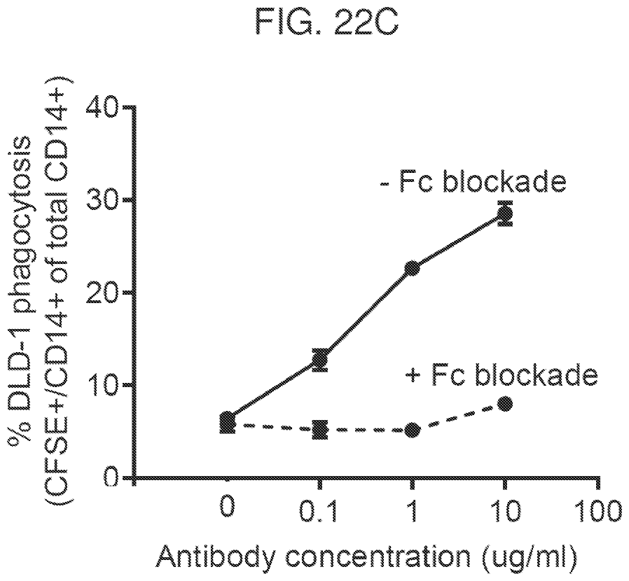

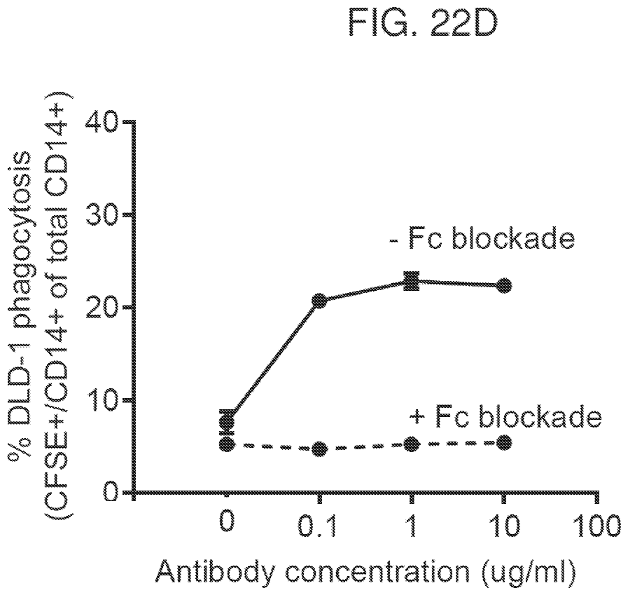

Human monocyte-derived macrophages were derived from leukapheresis of healthy human peripheral blood and incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, a cocktail of human Fc antibodies consisting of function blocking antibodies against human CD16 (clone 3G8, Invitrogen), CD32 (clone AT10, Invitrogen) and CD64 (clone 10.1, Invitrogen) were added, each at a final concentration of 10 μg/ml (+Fc block). 30 μg/ml mIgG1 (Biolegend) was used as an isotype control (−Fc block). Immediately afterwards the targeted human cancer cells Jurkat (FIG. 22A and FIG. 22B) or DLD-1 (FIG. 22C and FIG. 22D) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP mAbs SIRP4 (FIG. 22A and FIG. 22C) or SIRP9 (FIG. 22B and FIG. 22D) were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺, indicating complete phagocytosis.

To dissect the effect of individual Fc receptors, phagocytosis was performed as above, but Fc blocking antibodies were added individually at 10 μg/ml and compared to control mIgG1 at 10 μg/ml.

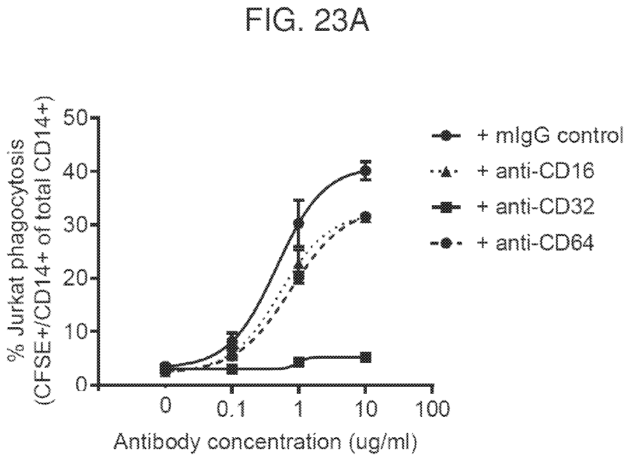

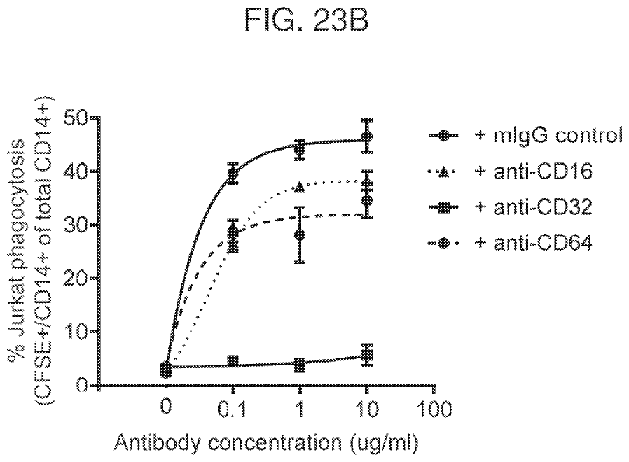

As shown in FIG. 22A and FIG. 22B, the soluble anti-SIRP mAbs SIRP4 and SIRP9 induced phagocytosis of Jurkat cells by human macrophages but the phagocytic activity was abrogated by the addition of Fc blocking antibody cocktail. The same effect was observed with SIRP4 (FIG. 22C) and SIRP9 (FIG. 22D) when DLD-1 cells were used as target cells. As shown in FIG. 23, functional blockade of CD32 (FcγRII) but not CD16 or CD64 inhibited both SIRP4 (FIG. 23A) and SIRP9 (FIG. 23B) induced phagocytosis of Jurkat T-ALL cells. This is consistent with the known isotype interactions with Fc receptors.

### Example 26

### Anti-SIRP Antibodies do not Induce Phagocytosis of Normal Autologous Human Peripheral Blood Mononuclear Cells

To assess the effect of anti-SIRP antibodies of the present disclosure on the phagocytosis of normal, non-cancerous cells in vitro, the following method was employed using flow cytometry.

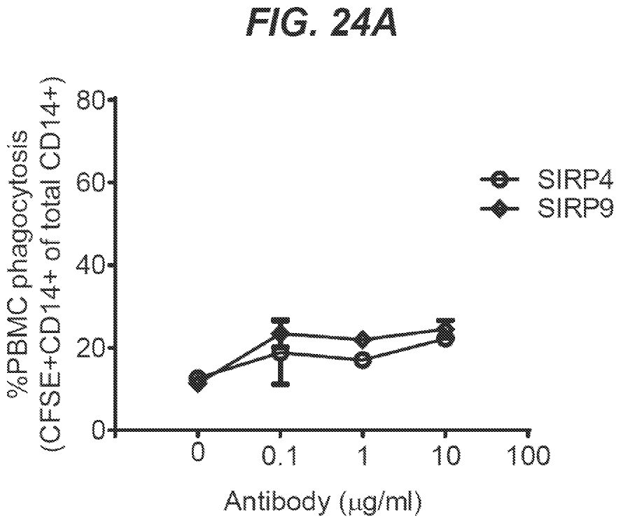

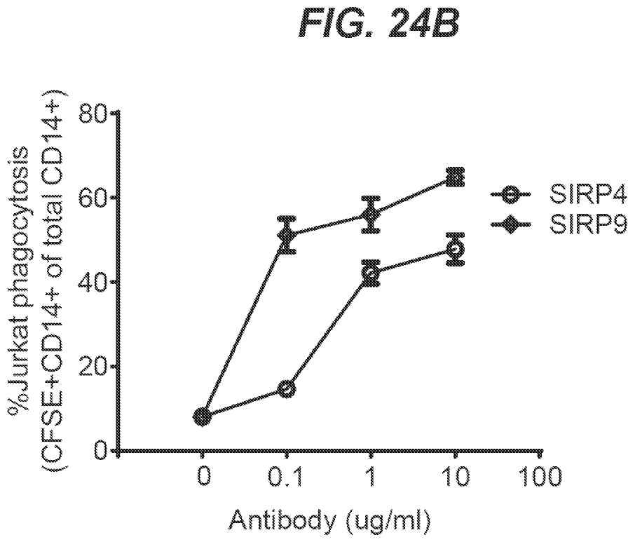

Human monocyte-derived macrophages were derived from leukapheresis of healthy human peripheral blood. To generate the macrophages, CD14⁺ monocytes were incubated in AIM-V media (Life Technologies) supplemented with 50 ng/ml M-CSF (Biolegend) for seven days. For the in vitro phagocytosis assay, macrophages were re-plated at a concentration of 3×10⁴ cells per well in 100 μl of AIM-V media supplemented with 50 ng/ml M-CSF in a 96-well plate and allowed to adhere for 24 hours. Once the effector macrophages adhered to the culture dish, normal autologous human peripheral blood mononuclear cells (FIG. 24A) or Jurkat T-ALL cells (FIG. 24B) were labeled with 1 μM 5(6)-Carboxyfluorescein diacetate N-succinimidyl ester (CFSE; Sigma Aldrich) and added to the macrophage cultures at a concentration of 8×10⁴ cells in 100 μl of AIM-V media without supplements. Anti-SIRP antibodies SIRP4 or SIRP9 were added at various concentrations immediately upon mixture of target and effector cells and allowed to incubate at 37° C. for 3 hours. After 3 hours, all non-phagocytosed cells were removed, and the remaining cells washed three times with PBS. Cells were then incubated in Accutase (Stemcell Technologies) to detach macrophages, collected into microcentrifuge tubes, and incubated in 100 ng of allophycocyanin (APC) labeled CD14 antibodies (BD biosciences) for 30 minutes, washed once, and analyzed by flow cytometry (Attune, Life Technologies) for the percentage of CD14⁺ cells that were also CFSE⁺, indicating complete phagocytosis.

As shown in FIG. 24A, the soluble anti-SIRP antibodies SIRP4 and SIRP9 did not induce phagocytosis of PBMCs by human macrophages. In contrast, as shown in FIG. 24B, SIRP4 and SIRP9 induced Jurkat T-ALL cell phagocytosis by macrophages in a dose dependent manner.

1. A method of treating cancer in a subject in need thereof, the method comprising administering to the subject a monoclonal antibody or antigen binding fragment thereof which specifically binds human SIRPα in an amount effective to treat cancer,
wherein the monoclonal antibody or antigen binding fragment thereof induces phagocytosis of cancer cells without inducing phagocytosis of normal peripheral blood mononuclear cells (PBMCs),
wherein the monoclonal antibody or antigen binding fragment comprises three light-chain complementarity determining regions (LCDR1, LCDR2, LCDR3) selected from:
i. SEQ ID NO:1, SEQ ID NO:2, SEQ ID NO:3;
ii. SEQ ID NO:4, SEQ ID NO:5, SEQ ID NO:6;
iii. SEQ ID NO:7, SEQ ID NO:8, SEQ ID NO:9;
iv. SEQ ID NO:10, SEQ ID NO:11, SEQ ID NO:12;
v. SEQ ID NO:13, SEQ ID NO:14, SEQ ID NO: 15;
vi. SEQ ID NO:16, SEQ ID NO:17, SEQ ID NO: 18;
vii. SEQ ID NO:19, SEQ ID NO:20, SEQ ID NO:21;
viii. SEQ ID NO:22, SEQ ID NO:23, SEQ ID NO:24;
ix. SEQ ID NO:25, SEQ ID NO:26, SEQ ID NO:27;
x. SEQ ID NO:28, SEQ ID NO:29, SEQ ID NO:30;
xi. SEQ ID NO:10, SEQ ID NO:31, SEQ ID NO:12;
xii. SEQ ID NO:10, SEQ ID NO:31, SEQ ID NO:32; and
xiii. SEQ ID NO:25, SEQ ID NO:26, SEQ ID NO:27; and
three heavy-chain complementarity determining regions (HCDR1, HCDR2, HCDR3) selected from:
xiv. SEQ ID NO:33, SEQ ID NO:34, and SEQ ID NO:35;
xv. SEQ ID NO:36, SEQ ID NO:37, and SEQ ID NO:38;
xvi. SEQ ID NO:39, SEQ ID NO:40, and SEQ ID NO:41;
xvii. SEQ ID NO:42, SEQ ID NO:43, and SEQ ID NO:44;
xviii. SEQ ID NO:45, SEQ ID NO:46, and SEQ ID NO:47;
xix. SEQ ID NO:48, SEQ ID NO:49, and SEQ ID NO:50;
xx. SEQ ID NO:51, SEQ ID NO:52, and SEQ ID NO:53;
xxi. SEQ ID NO:54, SEQ ID NO:55, and SEQ ID NO:56;
xxii. SEQ ID NO:57, SEQ ID NO:58, and SEQ ID NO:59;
xxiii. SEQ ID NO:60, SEQ ID NO:61, and SEQ ID NO: 62; and
xxiv. SEQ ID NO:57, SEQ ID NO:58, and SEQ ID NO: 63.

2. The method according to claim 18, wherein the monoclonal antibody or antigen binding fragment thereof which specifically binds human SIRPα comprises a heavy chain variable domain (V_{H}) and a light chain variable domain (V_{L}) selected from:
i. a heavy chain variable domain comprising the amino acid sequence of SEQ ID NO:81 and a light chain variable domain comprising the amino acid sequence SEQ ID NO:64;
ii. a heavy chain variable domain comprising the amino acid sequence of SEQ ID NO:82 and a light chain variable domain comprising the amino acid sequence SEQ ID NO:65;
iii. a heavy chain variable domain comprising the amino acid sequence of SEQ ID NO:83 and a light chain variable domain comprising the amino acid sequence SEQ ID NO:66;
iv. a heavy chain variable domain comprising the amino acid sequence of SEQ ID NO:84 and a light chain variable domain comprising the amino acid sequence SEQ ID NO:67;
v. a heavy chain variable domain comprising the amino acid sequence of SEQ ID NO:85 and a light chain variable domain comprising the amino acid sequence SEQ ID NO:68;
vi. a heavy chain variable domain comprising the amino acid sequence of SEQ ID NO:86 and a light chain variable domain comprising the amino acid sequence SEQ ID NO:69;
vii. a heavy chain variable domain comprising the amino acid sequence of SEQ ID NO:87 and a light chain variable domain comprising the amino acid sequence SEQ ID NO:70;
viii. a heavy chain variable domain comprising the amino acid sequence of SEQ ID NO:88 and a light chain variable domain comprising the amino acid sequence SEQ ID NO:71;
ix. a heavy chain variable domain comprising the amino acid sequence of SEQ ID NO:89 and a light chain variable domain comprising the amino acid sequence SEQ ID NO:72;
x. a heavy chain variable domain comprising the amino acid sequence of SEQ ID NO:90 and a light chain variable domain comprising the amino acid sequence SEQ ID NO:73;
xi. a heavy chain variable domain comprising the amino acid sequence of SEQ ID NO:91 and a light chain variable domain comprising the amino acid sequence SEQ ID NO:74;
xii. a heavy chain variable domain comprising the amino acid sequence of SEQ ID NO:91 and a light chain variable domain comprising the amino acid sequence SEQ ID NO:75;
xiii. a heavy chain variable domain comprising the amino acid sequence of SEQ ID NO:91 and a light chain variable domain comprising the amino acid sequence SEQ ID NO:76;
xiv. a heavy chain variable domain comprising the amino acid sequence of SEQ ID NO:92 and a light chain variable domain comprising the amino acid sequence SEQ ID NO:74;
xv. a heavy chain variable domain comprising the amino acid sequence of SEQ ID NO:92 and a light chain variable domain comprising the amino acid sequence SEQ ID NO:75;
xvi. a heavy chain variable domain comprising the amino acid sequence of SEQ ID NO:92 and a light chain variable domain comprising the amino acid sequence SEQ ID NO:76;
xvii. a heavy chain variable domain comprising the amino acid sequence of SEQ ID NO:93 and a light chain variable domain comprising the amino acid sequence SEQ ID NO:74;
xviii. a heavy chain variable domain comprising the amino acid sequence of SEQ ID NO:93 and a light chain variable domain comprising the amino acid sequence SEQ ID NO:75;
xix. a heavy chain variable domain comprising the amino acid sequence of SEQ ID NO:93 and a light chain variable domain comprising the amino acid sequence SEQ ID NO:76;
xx. a heavy chain variable domain comprising the amino acid sequence of SEQ ID NO:94 and a light chain variable domain comprising the amino acid sequence SEQ ID NO:74;
xxi. a heavy chain variable domain comprising the amino acid sequence of SEQ ID NO:94 and a light chain variable domain comprising the amino acid sequence SEQ ID NO:75;
xxii. a heavy chain variable domain comprising the amino acid sequence of SEQ ID NO:94 and a light chain variable domain comprising the amino acid sequence SEQ ID NO:76;
xxiii. a heavy chain variable domain comprising the amino acid sequence of SEQ ID NO:84 and a light chain variable domain comprising the amino acid sequence SEQ ID NO:77;
xxiv. a heavy chain variable domain comprising the amino acid sequence of SEQ ID NO:95 and a light chain variable domain comprising the amino acid sequence SEQ ID NO:78;
xxv. a heavy chain variable domain comprising the amino acid sequence of SEQ ID NO:95 and a light chain variable domain comprising the amino acid sequence SEQ ID NO:79;
xxvi. a heavy chain variable domain comprising the amino acid sequence of SEQ ID NO:95 and a light chain variable domain comprising the amino acid sequence SEQ ID NO:80;
xxvii. a heavy chain variable domain comprising the amino acid sequence of SEQ ID NO:96 and a light chain variable domain comprising the amino acid sequence SEQ ID NO:78;
xxviii. a heavy chain variable domain comprising the amino acid sequence of SEQ ID NO:96 and a light chain variable domain comprising the amino acid sequence SEQ ID NO:79;
xxix. a heavy chain variable domain comprising the amino acid sequence of SEQ ID NO:96 and a light chain variable domain comprising the amino acid sequence SEQ ID NO:80;
xxx. a heavy chain variable domain comprising the amino acid sequence of SEQ ID NO:97 and a light chain variable domain comprising the amino acid sequence SEQ ID NO:78;
xxxi. a heavy chain variable domain comprising the amino acid sequence of SEQ ID NO:97 and a light chain variable domain comprising the amino acid sequence SEQ ID NO:79;
xxxii. a heavy chain variable domain comprising the amino acid sequence of SEQ ID NO:97 and a light chain variable domain comprising the amino acid sequence SEQ ID NO:80; and
xxxiii. a heavy chain variable domain comprising the amino acid sequence of SEQ ID NO:89 and a light chain variable domain comprising the amino acid sequence SEQ ID NO:72.

3. The method according to claim 18, wherein the monoclonal antibody or antigen binding fragment thereof which specifically binds human SIRPα comprises one heavy chain and one light chain selected from:
i. a heavy chain comprising the amino acid sequence of SEQ ID NO:109 and a light chain comprising the amino acid sequence SEQ ID NO:98;
ii. a heavy chain comprising the amino acid sequence of SEQ ID NO:110 and a light chain comprising the amino acid sequence SEQ ID NO:99;
iii. a heavy chain comprising the amino acid sequence of SEQ ID NO:111 and a light chain comprising the amino acid sequence SEQ ID NO:100;
iv. a heavy chain comprising the amino acid sequence of SEQ ID NO:112 and a light chain comprising the amino acid sequence SEQ ID NO:101;
v. a heavy chain comprising the amino acid sequence of SEQ ID NO:112 and a light chain comprising the amino acid sequence SEQ ID NO:102;
vi. a heavy chain comprising the amino acid sequence of SEQ ID NO:112 and a light chain comprising the amino acid sequence SEQ ID NO:103;
vii. a heavy chain comprising the amino acid sequence of SEQ ID NO:113 and a light chain comprising the amino acid sequence SEQ ID NO:101;
viii. a heavy chain comprising the amino acid sequence of SEQ ID NO:113 and a light chain comprising the amino acid sequence SEQ ID NO:102;
ix. a heavy chain comprising the amino acid sequence of SEQ ID NO:113 and a light chain comprising the amino acid sequence SEQ ID NO:103;
x. a heavy chain comprising the amino acid sequence of SEQ ID NO:114 and a light chain comprising the amino acid sequence SEQ ID NO:101;
xi. a heavy chain comprising the amino acid sequence of SEQ ID NO:114 and a light chain comprising the amino acid sequence SEQ ID NO:102;
xii. a heavy chain comprising the amino acid sequence of SEQ ID NO:114 and a light chain comprising the amino acid sequence SEQ ID NO:103;
xiii. a heavy chain comprising the amino acid sequence of SEQ ID NO:115 and a light chain comprising the amino acid sequence SEQ ID NO:101;
xiv. a heavy chain comprising the amino acid sequence of SEQ ID NO:115 and a light chain comprising the amino acid sequence SEQ ID NO:102;
xv. a heavy chain comprising the amino acid sequence of SEQ ID NO:115 and a light chain comprising the amino acid sequence SEQ ID NO:103;
xvi. a heavy chain comprising the amino acid sequence of SEQ ID NO:116 and a light chain comprising the amino acid sequence SEQ ID NO:104;
xvii. a heavy chain comprising the amino acid sequence of SEQ ID NO:117 and a light chain comprising the amino acid sequence SEQ ID NO:105;
xviii. a heavy chain comprising the amino acid sequence of SEQ ID NO:117 and a light chain comprising the amino acid sequence SEQ ID NO:106;
xix. a heavy chain comprising the amino acid sequence of SEQ ID NO:117 and a light chain comprising the amino acid sequence SEQ ID NO:107;
xx. a heavy chain comprising the amino acid sequence of SEQ ID NO:118 and a light chain comprising the amino acid sequence SEQ ID NO:105;
xxi. a heavy chain comprising the amino acid sequence of SEQ ID NO:118 and a light chain comprising the amino acid sequence SEQ ID NO:106;
xxii. a heavy chain comprising the amino acid sequence of SEQ ID NO:118 and a light chain comprising the amino acid sequence SEQ ID NO:107;
xxiii. a heavy chain comprising the amino acid sequence of SEQ ID NO:119 and a light chain comprising the amino acid sequence SEQ ID NO:105;
xxiv. a heavy chain comprising the amino acid sequence of SEQ ID NO:119 and a light chain comprising the amino acid sequence SEQ ID NO:106;
xxv. a heavy chain comprising the amino acid sequence of SEQ ID NO:119 and a light chain comprising the amino acid sequence SEQ ID NO:107; and
xxvi. a heavy chain comprising the amino acid sequence of SEQ ID NO:120 and a light chain comprising the amino acid sequence SEQ ID NO:108.

4. The method according to claim 1, wherein the monoclonal antibody or antigen binding fragment thereof comprises an IgG isotype selected from IgG1, IgG1-N297Q, IgG2, IgG4, IgG4 S228P, IgG4 PE and variants thereof.

5. The method according to claim 1, wherein the monoclonal antibody or antigen binding fragment thereof binds human SIRPα and human SIRPγ.

6. The method according to claim 1, wherein the induction of phagocytosis is Fc-dependent.

7. The method according to claim 6, wherein the induction of phagocytosis is dependent on FcγR.

8. The method according to claim 7, wherein the FcγR is chosen from FcγRI (CD64), FcγRIIA (CD32), FcγRII1B (CD32), FcγRIIIA (CD16a), and FcγRIIIB (CD16b).

9. The method according to claim 1, wherein the monoclonal antibody or antigen binding fragment thereof is administered in combination with a chemotherapeutic agent or therapeutic antibody.

10. The method according to claim 9, wherein the therapeutic antibody is directed against a cellular target selected from CD47 (Cluster of Differentiation 47), CD70 (Cluster of Differentiation 70), CD200 (OX-2 membrane glycoprotein, Cluster of Differentiation 200), CD154 (Cluster of Differentiation 154, CD40L, CD40 ligand, Cluster of Differentiation 40 ligand), CD223 (Lymphocyte-activation gene 3, LAG3, Cluster of Differentiation 223), KIR (Killer-cell immunoglobulin-like receptors), GITR (TNFRSF18, glucocorticoid-induced TNFR-related protein, activation-inducible TNFR family receptor, AITR, Tumor necrosis factor receptor superfamily member 18), CD20 (Cluster of Differentiation), CD28 (Cluster of Differentiation 28), CD40 (Cluster of Differentiation 40, Bp50, CDW40, TNFRSF5, Tumor necrosis factor receptor superfamily member 5, p50), CD86 (B7-2, Cluster of Differentiation 86), CD160 (Cluster of Differentiation 160, BY55, NK1, NK28), CD258 (LIGHT, Cluster of Differentiation 258, Tumor necrosis factor ligand superfamily member 14, TNFSF14, herpesvirus entry mediator ligand (HVEM-L), CD270 (HVEM, Tumor necrosis factor receptor superfamily member 14, herpesvirus entry mediator, Cluster of Differentiation 270, LIGHTR, HVEA), CD275 (ICOSL, ICOS ligand, Inducible T-cell co-stimulator ligand, Cluster of Differentiation 275), CD276 (B7-H3, B7 homolog 3, Cluster of Differentiation 276), OX40L (OX40 Ligand), B7-H4 (B7 homolog 4, VTCN1, V-set domain-containing T-cell activation inhibitor 1), GITRL (Glucocorticoid-induced tumor necrosis factor receptor-ligand, glucocorticoid-induced TNFR-ligand), 4-1BBL (4-1BB ligand), CD3 (Cluster of Differentiation 3, T3D), CD25 (IL2Rα, Cluster of Differentiation 25, Interleukin-2 Receptor a chain, IL-2 Receptor a chain), CD48 (Cluster of Differentiation 48, B-lymphocyte activation marker, BLAST-1, signaling lymphocytic activation molecule 2, SLAMF2), CD66a (Ceacam-1, Carcinoembryonic antigen-related cell adhesion molecule 1, biliary glycoprotein, BGP, BGP1, BGPI, Cluster of Differentiation 66a), CD80 (B7-1, Cluster of Differentiation 80), CD94 (Cluster of Differentiation 94), NKG2A (Natural killer group 2A, killer cell lectin-like receptor subfamily D member 1, KLRD1), CD96 (Cluster of Differentiation 96, TActILE, T-cell activation increased late expression), CD112 (PVRL2, nectin, Poliovirus receptor-related 2, herpesvirus entry mediator B, HVEB, nectin-2, Cluster of Differentiation 112), CD115 (CSF1R, Colony stimulating factor 1 receptor, macrophage colony-stimulating factor receptor, M-CSFR, Cluster of Differentiation 115), CD205 (DEC-205, LY75, Lymphocyte antigen 75, Cluster of Differentiation 205), CD226 (DNAM1, Cluster of Differentiation 226, DNAX Accessory Molecule-1, PTA1, platelet and T-cell activation antigen 1), CD244 (Cluster of Differentiation 244, Natural killer cell receptor 2B4), CD262 (DR5, TrailR2, TRAIL-R2, Tumor necrosis factor receptor superfamily member 10b, TNFRSF10B, Cluster of Differentiation 262, KILLER, TRICK2, TRICKB, ZTNFR9, TRICK2A, TRICK2B), CD284 (Toll-like Receptor-4, TLR4, Cluster of Differentiation 284), CD288 (Toll-like Receptor-8, TLR8, Cluster of Differentiation 288), Leukemia Inhibitor Factor (LIF), TNFSF15 (Tumor necrosis factor superfamily member 15, Vascular endothelial growth inhibitor, VEGI, TL1A), TDO2 (Tryptophan 2,3-dioxygenase, TPH2, TRPO), IGF-1R (Type 1 Insulin-like Growth Factor), GD2 (Disialoganglioside 2), TMIGD2 (Transmembrane and immunoglobulin domain-containing protein 2), RGMB (RGM domain family, member B), VISTA (V-domain immunoglobulin-containing suppressor of T-cell activation, B7-H5, B7 homolog 5), BTNL2 (Butyrophilin-like protein 2), Btn (Butyrophilin family), TIGIT (T-cell Immunoreceptor with Ig and ITIM domains, Vstm3, WUCAM), Siglecs (Sialic acid binding Ig-like lectins), i.e., SIGLEC-15, Neurophilin, VEGFR (Vascular endothelial growth factor receptor), ILT family (LIRs, immunoglobulin-like transcript family, leukocyte immunoglobulin-like receptors), NKG families (Natural killer group families, C-type lectin transmembrane receptors), MICA (MHC class I polypeptide-related sequence A), TGFβ (Transforming growth factor β), STING pathway (Stimulator of interferon gene pathway), Arginase (Arginine amidinase, canavanase, L-arginase, arginine transamidinase), EGFRvIII (Epidermal growth factor receptor variant III), HHLA2 (B7-H7, B7y, HERV-H LTR-associating protein 2, B7 homolog 7), inhibitors of PD-1 (Programmed cell death protein 1, PD-1, CD279, Cluster of Differentiation 279), PD-L1 (B7-H1, B7 homolog 1, Programmed death-ligand 1, CD274, cluster of Differentiation 274), PD-L2 (B7-DC, Programmed cell death 1 ligand 2, PDCD1LG2, CD273, Cluster of Differentiation 273), CTLA-4 (Cytotoxic T-lymphocyte-associated protein 4, CD152, Cluster of Differentiation 152), BTLA (B- and T-lymphocyte attenuator, CD272, Cluster of Differentiation 272), Indoleamine 2,3-dioxygenase (IDO, IDO1), TIM3 (HAVCR2, Hepatitis A virus cellular receptor 2, T-cell immunoglobulin mucin-3, KIM-3, Kidney injury molecule 3, TIMD-3, T-cell immunoglobulin mucin-domain 3), A2A adenosine receptor (ADO receptor), CD39 (ectonucleotide triphosphate diphosphohydrolase-1, Cluster of Differentiation 39, ENTPD1), CD73 (Ecto-5′-nucleotidase, 5′-nucleotidase, 5′-NT, Cluster of Differentiation 73), CD27 (Cluster of Differentiation 27), ICOS (CD278, Cluster of Differentiation 278, Inducible T-cell Co-stimulator), CD137 (4-1BB, Cluster of Differentiation 137, tumor necrosis factor receptor superfamily member 9, TNFRSF9), OX40 (CD134, Cluster of Differentiation 134), TNFSF25 (Tumor necrosis factor receptor superfamily member 25), IL-10 (Interleukin-10, human cytokine synthesis inhibitory factor, CSIF), PVRIG (Poliovirus receptor-related immunoglobulin domain-containing protein), and Galectins.

11. The method according to claim 1, wherein the monoclonal antibody or antigen binding fragment thereof is administered in combination with an opsonizing antibody which targets an antigen on a tumor cell.

12. The method according to claim 11, wherein the opsonizing antibody is selected from one or more of anti-CD20, anti-HER2, anti-CD52, anti-EGFR, anti-RANKL, anti-SLAMF7, anti-PD-L1, anti-CD38, anti-CD19/CD3, and anti-GD2 antibodies.

13. The method according to claim 12, wherein the opsonizing antibody is selected from one or more of rituximab, trastuzumab, alemtuzumab, cetuximab, panitumumab, ofatumumab, denosumab, pertuzumab, panitumumab, elotuzumab, atezolizumab, avelumab, durvalumab, necitumumab, daratumumab, obinutuzumab, blinatumomab, and dinutuximab.

14. The method according to claim 12, wherein the i-s-opsonizing antibody is selected from one or more of anti-CD20, anti-EGFR, anti-PD-1, and anti-PD-L1 antibodies.

15. The method according to claim 1, wherein said cancer is selected from leukemia, lymphoma, multiple myeloma, ovarian cancer, breast cancer, endometrial cancer, colon cancer (colorectal cancer), rectal cancer, bladder cancer, urothelial cancer, lung cancer, bronchial cancer, bone cancer, prostate cancer, pancreatic cancer, gastric cancer, hepatocellular carcinoma, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, cancer of uterus, glioblastoma, meduloblastoma, astrocytoma, glioma, meningioma, gastrinoma, neuroblastoma, melanoma, myelodysplastic syndrome, and a sarcoma.

16. The method according to claim 15,
wherein said leukemia is selected from systemic mastocytosis, acute lymphocytic (lymphoblastic) leukemia (ALL), T-cell ALL, acute myeloid leukemia (AML), chronic lymphocytic leukemia (CLL), chronic myeloid leukemia (CML), myeloproliferative disorder/neoplasm, myelodysplastic syndrome, monocytic cell leukemia, and plasma cell leukemia;
wherein said lymphoma is selected from histiocytic lymphoma and T-cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia;
wherein said sarcoma is selected from osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma; and
wherein said lung cancer is selected from non-small cell lung cancer, adenocarcinoma of the lung, squamous cell carcinoma of the lung.

17. (canceled)

18. (canceled)

19. The method according to claim 1, wherein the monoclonal antibody or antigen binding fragment thereof is pan-allelic.

20. The method according to claim 1, wherein the monoclonal antibody or antigen binding fragment thereof affects one or more of SIRPα dimerization, clustering and architecture.

21. The method according to claim 1, wherein the monoclonal antibody or antigen binding fragment thereof causes a reduction of cell surface SIRPα.

22. The method according to claim 21, wherein the cell surface SIRPα is internalized.
